(19) United States
(12) Patent Application Publication
(10) Pub. No.: US 2024/0390282 A1
(43) Pub. Date: Nov. 28, 2024

(54) DRUG-LOADED LIPOSOMES AND USES THEREOF IN TREATING BIOFILM

(71) Applicant: Yeda Research and Development Co. Ltd., Rehovot (IL)

(72) Inventors: Jacob KLEIN, Rehovot (IL); Ronit GOLDBERG, Rehovot (IL); Yaara OPPENHEIMER-SHAANAN, Rehovot (IL); Weifeng LIN, Rehovot (IL); Monika KLUZEK, Rehovot (IL)

(73) Assignee: Yeda Research and Development Co. Ltd., Rehovot (IL)

(21) Appl. No.: 18/792,590

(22) Filed: Aug. 2, 2024

Related U.S. Application Data
(63) Continuation of application No. PCT/IL2023/050121, filed on Feb. 2, 2023.
(60) Provisional application No. 63/306,156, filed on Feb. 3, 2022.

(51) Int. Cl. A61K 9/127; A61K 31/352; A61K 31/635; A61P 31/04

(52) U.S. Cl. CPC A61K 9/1271; A61K 31/352; A61K 31/635; A61P 31/04

(57) ABSTRACT

Liposomes, and pharmaceutical compositions comprising same, for use in treating biofilm in a subject in need thereof are disclosed herein. The liposomes comprise a bilayer-forming lipid; a polymer-lipid conjugate having the general formula I, as described and defined in the specification; a positively-charged lipidic agent, incorporated within a lipid bilayer and/or on a surface of the liposome; and a therapeutically active agent, bound to a surface of the liposome and/or within a lipid bilayer and/or core of the liposome.

### RELATED APPLICATIONS

This application is a Continuation of PCT Patent Application No. PCT/IL2023/050121 having International filing date of Feb. 2, 2023, which claims the benefit of priority under 35 USC § 119(e) of U.S. Provisional Patent Application No. 63/306,156 filed on Feb. 3, 2022. The contents of the above applications are all incorporated by reference as if fully set forth herein in their entirety.

### FIELD AND BACKGROUND OF THE INVENTION

The present invention, in some embodiments thereof, relates to therapy, and more particularly, but not exclusively, to drug-loaded liposomal compositions usable, inter alia, for treating (e.g., interfering with a formation of, disrupting and/or eradicating) biofilm in a subject in need thereof.

Nano-vehicles, prominently including liposomes, have been extensively studied as drug carriers in several contexts [Mitchell et al. Nat Rev Drug Discov 2021, 20 (2), 101-124], but bacterial infections forming surface-adherent microbial communities, or biofilms, common in living tissues and on synthetic surfaces, are notoriously resistant to delivery of antimicrobial agents [Koo et al. Nat Rev Microbiol 2017, 15 (12), 740; Sharma et al. Antimicrob Resist Infect Control 2019, 8 (1), 76]. This is due to the biofilm's highly developed and adaptive defense and communication mechanisms [Costerton et al. Science 1999, 284 (5418), 1318-1322; Kember et al. Pathogens 2022, 11 (2), 220], which act partly via physical barriers limiting the penetration thereto, as well as limited cell entry due to low membrane permeability [Kember et al. 2022, supra; and Davies, D. Nat Rev Drug Discov 2003, 2 (2), 114-122].

Key requirements to enhance biofilm eradication [Ramos et al. Int J Nanomed 2018, 13, 1179-1213] are (a) the development of new nano-delivery systems that can effectively penetrate the structure of the biofilm; and subsequently (b) release their drug cargo directly into the biofilm-embedded cells.

To date, the leading drug-vectors of choice fulfilling these requirements are lipid vesicles, or liposomes [Ulrich et al. Bioscience Rep 2002, 22 (2), 129-150], due to their low immunogenicity, firm safety profiles [Torchilin et al. Nat Rev Drug Discov 2005, 4 (2), 145-160], and the ability to encapsulate both lipophilic and hydrophilic compounds [Sarfraz et al. Pharm 2018, 10 (3), 151; Vargason et al. Nat Biomed Eng 2021, 1-17; and Bozzuto and Molinari, Int J Nanomed 2015, 10 (1), 975-999].

Indeed, the usage of liposomes has significantly improved the therapeutic index for a range of biomedical applications by stabilizing active agents, overcoming obstacles to cellular and tissue uptake, and improving biodistribution of compounds to target sites in vivo, while exhibiting favorable safety profile [Bozzuto and Molinari, 2015, supra]. Despite being advantageous drug nanocarriers, liposomes have (a) an intrinsically low colloidal stability, which results in short shelf-life; and (b) low penetration through the biofilm matrix, which limits their efficacy in eradicating biofilms [Rukavina and Vanić, Pharm 2016, 8 (2), 18].

The most common strategy utilized to improve these drawbacks is to introduce surface functionalization via conjugation of hydrophilic polymers, such as polyethylene glycol (PEG), to the surface of the carriers to obtain sterically-stabilized liposomes [Pasut and Veronese, J Control Release 2012, 161 (2), 461-472; and Milla et al. Curr Drug Metab 2012, 13 (1), 105-119]. However, PEG-functionalizations (PEGylations) drastically reduce interaction with the target cells [Verhoef and Anchordoquy, Drug Deliv Transl Re 2013, 3 (6), 499-503; Soenen et al. Part Syst Char 2014, 31 (7), 794-800; and Hatakeyama et al. Biological Pharm Bulletin 2013, 36 (6), 892-899], and therefore significantly limit the applicability of liposomes in biofilm eradication [Ahmed et al. Colloids Surfaces Physicochem Eng Aspects 2001, 194 (1-3), 287-296].

WO 2016/051413 discloses methods of inhibiting biofilm formation and of inhibiting adsorption of a biofouling-promoting agent on the surface of a substrate using a composition comprising liposomes. The liposomes and hydrogel-containing matrix taught therein reduce absorption of biofouling-promoting agents and thus impart anti-biofouling (ABF) properties to the substrate.

WO 2017/109784 discloses polymeric compounds, lipid bilayers comprising bilayer-forming lipid and the polymeric compound, liposomes made therefrom and uses thereof. The disclosed liposomes exhibit improved stability and demonstrate enhanced performance as lubricants compared to, for example, corresponding PEG-functionalized liposomes.

WO 2018/150429 discloses liposomes such as described herein in WO 2017/109784 which are usable as drug-delivery vehicles.

Additional background art includes Lin et al. Langmuir 2019, 35 (18), 6048-6054; Lin et al. J Mater Chem B 2022, 10, 2820; Zhang et al. Nano Res 2016, 9 (8), 2424-2432; Adler et al. J Mater Chem B 2021, 10, 2512; Cao et al. Acs Macro Lett 2019, 8 (6), 651-657; Liu et al. J Mater Chem B 2020, 8 (20), 4395-4401; Ramos et al. Int J Nanomed 2018, 13, 1179-1213; Drulis-Kawa et al. Int J Pharmaceut 2009, 367 (1-2), 211-219; Nicolosi et al. Int J Antimicrob Ag 2010, 35 (6), 553-558; Ma et al. Int J Nanomed 2013, 8 (1), 2351-2360; Gharib et al. J Pharm Sci 2012, 20 (1), 41; Drulis-Kawa et al. Cellular & Molecular Biology Letters, 2006, 11 (3), 360-375; Webb et al. Biochimica Et Biophysica Acta Bba-Biomembr 1995, 1238 (2), 147-155; Zhang et al. Proc National Acad Sci 2018, 115 (13), 201722323; Szabo, G., Nature 1974, 252 (5478), 47-49; Al-Rekabi and Contera, Proc National Acad Sci 2018,115 (11), 201719065; Melcrová et al. Sci Rep-uk 2016, 6 (1), srep38035; and Kluzeket al. ACS Nano 2022, 16 (10), 15792-15804.

### SUMMARY OF THE INVENTION

According to an aspect of some embodiments of the present invention, there is provided a liposome comprising: (a) at least one bilayer-forming lipid; (b) a polymeric compound having the general formula I:

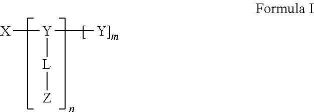

wherein:
m is zero or a positive integer;
n is an integer which is at least 1, wherein when X does not comprise a phosphate group, n is at least 2;
X is a lipid moiety;
Y is a backbone unit which forms a polymeric backbone;
L is absent or is a linking moiety; and
Z has the general formula II:

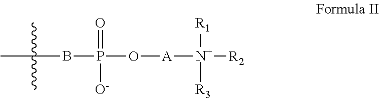

wherein:
A is a substituted or unsubstituted hydrocarbon;
B is an oxygen atom or is absent; and
R₁-R₃ are each independently selected from the group consisting of hydrogen, alkyl, cycloalkyl, heteroalicyclic, aryl and heteroaryl;
c) a positively-charged lipidic agent, incorporated within a lipid bilayer and/or on a surface of the liposome; and
(d) a therapeutically active agent, bound to a surface of the liposome and/or within a lipid bilayer and/or core of the liposome,
the liposome being for use in treating biofilm (interfering with a formation of, disrupting and/or eradicating) in a subject in need thereof.

According to some of any of the embodiments described herein, the liposome further comprises a sterol, bound to a surface of the liposome and/or within a lipid bilayer and/or core of the liposome.

According to some of any of the embodiments described herein, the sterol is cholesterol.

According to some of any of the embodiments described herein, the positively-charged lipidic agent comprises a hydrocarbon chain of from 4 to 30 carbon atoms in length, substituted and/or terminated by at least one substituent that is positively charged at physiological conditions.

According to some of any of the embodiments described herein, the substituent is or comprises an amine.

According to some of any of the embodiments described herein, the positively-charged lipidic agent is stearyl amine.

According to some of any of the embodiments described herein, a molar ratio of the bilayer-forming lipid and the positively-charged lipidic agent is in a range of from 1:1 to 100:1, or from 1:1 to 50:1 (e.g., 20:1).

According to some of any of the embodiments described herein, the liposome is characterized by a diameter in a range of from 10 nm to 1000 nm, or from 10 nm to 500 nm, or from 50 nm to 500 nm, or from 10 nm to 300 nm, or from 50 nm to 300 nm, or from 10 nm to 200 nm, or from 50 nm to 200 nm, or from 10 nm to 100 nm, or from 50 nm to 100 nm, or from 100 nm to 200 nm.

According to some of any of the embodiments described herein, a molar ratio of the bilayer-forming lipid and the polymeric compound is in a range of from 1:1 to 100:1, or from 5:1 to 50:1 (e.g., 20:1).

According to some of any of the embodiments described herein, a molar ratio of the positively-charged lipidic agent and the polymeric compound is in a range of from 10:1 to 1:10 (e.g., 1:1).

According to some of any of the embodiments described herein, Y is a substituted or unsubstituted alkylene unit.

According to some of any of the embodiments described herein, Y is a substituted or unsubstituted ethylene unit.

According to some of any of the embodiments described herein, Y has the formula —CR₄R₅—CR₆D—, wherein: when Y is a backbone unit which is not attached to the L or the Z, D is R₇; and when Y is a backbone unit which is attached to the L or the Z, D is a covalent bond or a linking group attaching Y to the L or the Z, the linking group being selected from the group consisting of —O—, —S—, alkylene, arylene, sulfinyl, sulfonyl, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, and amino; and R₄-R₇ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azide, azo, phosphate, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, and amino.

According to some of any of the embodiments described herein, R₄ and R₅ are each hydrogen.

According to some of any of the embodiments described herein, R₆ is hydrogen.

According to some of any of the embodiments described herein, the linking group is selected from the group consisting of —O—, —C(═O)O—, —C(═O)NH— and phenylene.

According to some of any of the embodiments described herein, the linking group is —C(═O)O—.

According to some of any of the embodiments described herein, L is a substituted or unsubstituted hydrocarbon from 1 to 10 carbon atoms in length.

According to some of any of the embodiments described herein, L is a substituted or unsubstituted ethylene group.

According to some of any of the embodiments described herein, B is an oxygen atom.

According to some of any of the embodiments described herein, A is a substituted or unsubstituted hydrocarbon from 1 to 4 carbon atoms in length.

According to some of any of the embodiments described herein, A is a substituted or unsubstituted ethylene group.

According to some of any of the embodiments described herein, R₁-R₃ are each independently hydrogen or C₁₋₄-alkyl.

According to some of any of the embodiments described herein, R₁-R₃ are each methyl.

According to some of any of the embodiments described herein, n is at least 3.

According to some of any of the embodiments described herein, n is in a range of from 5 to 50, and m is in a range of from 0 to 50.

According to some of any of the embodiments described herein, at least a portion of the Y, the L and/or the Z comprise at least one targeting moiety.

According to some of any of the embodiments described herein, the polymeric compound has the general formula Ib:

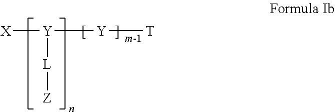

wherein:
T is a unit of the Y which comprises the at least one targeting moiety;
X and T are attached to distal termini of the polymeric compound; and
X, Y, L, Z, n and m are as defined for general formula I, with the proviso that m is a positive integer.

According to some of any of the embodiments described herein, X has the general formula III:

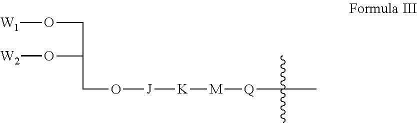

wherein:
W₁ and W₂ are each independently selected from the group consisting of hydrogen, alkyl, alkenyl, alkynyl and acyl, wherein at least one of W₁ and W₂ is not hydrogen;
J is —P(═O)(OH)—O— or absent;
K is a substituted or unsubstituted hydrocarbon from 1 to 10 carbon atoms in length;
M is a linking group selected from the group consisting of —O—, —S—, amino, sulfinyl, sulfonyl, phosphate, phosphonyl, phosphinyl, carbonyl, thiocarbonyl, urea, thiourea, carbamyl, thiocarbamyl, amido, carboxy, and sulfonamide, or absent; and
Q is a substituted or unsubstituted hydrocarbon from 1 to 10 carbon atoms in length, or absent,
wherein when M is absent, Q is also absent.

According to some of any of the embodiments described herein, J is —P(═O)(OH)—O— and K is selected from the group consisting of an ethanolamine moiety, a serine moiety, a glycerol moiety and an inositol moiety.

According to some of any of the embodiments described herein, M is amido.

According to some of any of the embodiments described herein, Q is dimethylmethylene (—C(CH₃)₂—).

According to some of any of the embodiments described herein, J, M and Q are each absent.

According to some of any of the embodiments described herein, K is —C(═O)—C(CH₃)₂—.

According to some of any of the embodiments described herein, at least one of W₁ and W₂ is alkyl, alkenyl, alkynyl or acyl, being from 10 to 30 carbon atoms in length.

According to some of any of the embodiments described herein, the lipid moiety comprises at least one fatty acid moiety selected from the group consisting of lauroyl, myristoyl, palmitoyl, stearoyl, palmitoleoyl, oleoyl, and linoleoyl.

According to some of any of the embodiments described herein, the liposome is formulated as part of a pharmaceutical composition, which further comprises a pharmaceutically acceptable carrier.

According to some of any of the embodiments described herein, the therapeutically active agent is an antimicrobial agent effective in treating the biofilm.

According to some of any of the embodiments described herein, the biofilm is a bacterial biofilm.

According to some of any of the embodiments described herein, the bacterial biofilm is formed by a pathogenic bacterium selected from Pseudomonas, Escherichia, Klebsiella, Enterobacter, Acinetobacter, Serratia, Haemophilus, Chlamydia, Salmonella, Arsenophonus, Cosenzaea, Moraxella, Brucella, Bordetella, Vibrio, Campylobacter, Legionella, Francisella, Photorhabdus, Neisseria, Proteus, Shigella, Edwardsiella, Plesiomonas, Aeromonas, Alcaligenes, Providencia, Yersinia, Staphylococcus, Bacillus, Listeria, Streptococcus, Gardnerella, Cronobacter, Enterococcus, Clostridium, Corynebacterium, Mycobacterium, Micrococcus, Hafnia, Morganella, Pasteurella, Mycoplasma, Ureaplasma, Coxiella, Borrelia, Aerococcus, Lactococcus, Actinomyces, Rhodococcus, Propionibacterium, Bartonella, Lactobacillus, Bifidobacterium, Rothia, Porphyromonas, Prevotella, Bacteroides, Fusobacterium, Megasphaera, Acidaminococcus, Veilonella, Norcardia, Treponema, Leptospira, Deinococcus, Helicobacter, Burkholderia, Canipylobacter, Micropolyspora and Thermoactinomyces.

According to some of any of the embodiments described herein, the bacterial biofilm is a gram-negative bacterial biofilm.

According to some of any of the embodiments described herein, the gram-negative bacterial biofilm is formed by a pathogenic gram-negative bacterium selected from Pseudomonas, Escherichia, Klebsiella, Enterobacter, Acinetobacter, Serratia, Haemophilus, Chlamydia, Salmonella, Arsenophonus, Veilonella, Cosenzaea, Moraxella, Brucella, Bordetella, Vibrio, Campylobacter, Legionella, Francisella, Prevotella, Acidaminococcus, Megasphaera, Fusobacterium, Photorhabdus, Neisseria, Proteus, Shigella, Bacteroides, Porphyromonas, Edwardsiella, Plesiomonas, Aeromonas, Alcaligenes, Providencia, Pasteurella, Hafnia, Morganella, Mycoplasma, Ureaplasma, Coxiella, Leptospira, Treponema, Borrelia, Aerococcus, Lactococcus, Bartonella, Yersinia, Deinococcus, Helicobacter, Burkholderia, Canipylobacter, Micropolyspora and Thermoactinomyces.

According to some of any of the embodiments described herein, the bacterial biofilm is formed by Pseudomonas aeruginosa.

According to some of any of the embodiments described herein, the therapeutically active agent is an antibiotic selected from an aminoglycoside (e.g., gentamicin, tobramycin, amikacin, streptomycin), a fluoroquinolone (e.g., ciprofloxacin, levofloxacin, gatifloxacin, moxifloxacin), a carbapenem (e.g., imipenem, meropenem, ertapenem), a polymyxin (e.g., colistin, polymyxin B, polymyxin E), a tetracycline (e.g., tetracycline, doxycycline), a macrolide (e.g., azithromycin, clarithromycin, erythromycin), a beta-lactam (e.g., ampicillin, amoxicillin, ticarcillin, piperacillin, imipenem, oxacillin, cephalosporins), a sulfonamide (e.g., sulfamethoxazole, sulfadiazine, sulfisoxazole, sulfacetamide, sulfamethazine, sulfasalazine), a rifamycin (e.g., rifampin, rifabutin), a nitroimidazole (e.g., metronidazole, tinidazole), phosphonic acid antibiotics (e.g., fosfomycin), chloramphenicol, glycopeptides (e.g., vancomycin), oxazolidinones (e.g., linezolid), cephalosporin (e.g., cefazolin, ceftriaxone, cephalothin, ceftazidime, cefepime), monobactam (e.g., aztreonam), nitrofurans (e.g., nitrofurantoin) and lipopeptide (e.g., daptomycin).

According to some of any of the embodiments described herein, the therapeutically active agent is selected from Sulfamethoxazole (SMX) and Ellagic acid (EA).

According to some of any of the embodiments described herein, the liposome comprises at least two therapeutically active agents, each independently bound to a surface of the liposome and/or within a lipid bilayer and/or core of the liposome.

According to some of any of the embodiments described herein, the at least two therapeutically active agents act in synergy.

According to an aspect of some embodiments of the present invention, pharmaceutical composition for use in treating biofilm in a subject in need thereof, the composition comprising a plurality of liposomes, wherein in at least a portion of the liposomes each liposome is a liposome as described herein in any of the respective embodiments and any combination thereof.

According to some of any of the embodiments described herein, in at least one portion of the liposomes each liposome is a liposome as described in any one of claims 1 to 45, comprising a first therapeutically active agent bound to a surface of the liposome and/or within a lipid bilayer and/or core of the liposome, and in at least one another portion of the liposomes each liposome is a liposome as described in any one of claims 1 to 45, comprising a second therapeutically active agent bound to a surface of the liposome and/or within a lipid bilayer and/or core of the liposome, the first and second therapeutically active agents being different from one another.

According to some of any of the embodiments described herein, the first and second therapeutically active agents act in synergy.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of the exemplary pMPC-functionalized liposomes, showing a pMPC conjugated to phosphatidylethanolamine (pMPC-conjugated distearylphosphorylethanolamine, DSPE) lipid, which is present at the liposome surface at 5% (mol/mol). The pMPCylated liposomes are further composed of hydrogenated soy phosphatidylcholine lipid (HSPC) with 40% mol/mol cholesterol and 5% (mol/mol) stearylamine (SA). Liposomes were loaded with exemplary antimicrobial agents: sulfamethoxazole (SMX) alone or in combination with ellagic acid (EA).

FIG. 2A are comparative plots presenting changes in hydrodynamic diameter of HSPC (50%)/cholesterol (40%)/stearylamine (5%)/pMPC (5 kDa, 5%) liposomes which were extruded through 200 nm pore membrane, following storage at 4° C. for up to 24 weeks.

FIGS. 2B-C are comparative plots presenting changes in hydrodynamic diameter of non-functionalized liposomes composed of (FIG. 2B) HSPC (60%)/cholesterol (40%); and (FIG. 2C) HSPC (55%)/cholesterol (40%)/stearylamine (5%), obtained following extruded through 200 nm pore membrane, following storage at 4° C. for up to 5 days.

FIG. 2D is a photograph of non-functionalized liposomes composed of HSPC (60%)/cholesterol (40%) (left Eppendorf) and same composition doped with 5% stearylamine (right Eppendorf). Both samples were calcein-loaded and images were taken 3 days post-extrusion upon storage at 4° C.

FIG. 3 is a bar graph presenting cell viability of HEK293 cells upon a 24-hour incubation period with exemplary antimicrobial agents (SMX and SMX/EA), as free drugs and when loaded in the exemplary pMPC—functionalized liposomes described in FIG. 1 and in corresponding PEG-functionalized liposomes. Liposomes were extruded through 200 nm pore membrane. Cell viability was measured with a Cell Proliferation Kit (XTT based) following the manufacturer instruction. The data represents the averages and standard deviations from two independent biological repeats. NT=not treated.

FIGS. 4A-C are representative cryo-TEM images of unloaded pMPCylated LUVs after 1 hour incubation in 10 Mm (FIG. 4A), and 40 mM (FIG. 4B) calcium acetate (Ca(Ac)₂) solution and of corresponding PEG-LUVs (FIG. 4C) in 40 mM solution of Ca(Ac)₂. White arrows indicate adhesion points and black arrow indicates no adhesion. Scale bar: 50 nm.

FIG. 4D is a bar graph presenting distance distribution between opposing vesicles measured based on cryo-TEM pictures. The distance between liposomes' membranes was measured considering the length between the phospholipid headgroups of the inner leaflet of the two vesicles. The data represent measurements extracted from 7 separate fields of view, for a total of N=45 for pMPC-LUVs in 10 mM Ca(Ac)₂ pMPC; N=43 for pMPC-LUVs in 40 mM Ca(Ac)₂ and N=44 for PEG-LUVs in 40 mM Ca(Ac)₂.

FIGS. 5A-B are confocal microscopy images of time-lapse acquisition of pMPC-giant unilamellar vesicles (GUVs) stained with DiI dye (red), interacting upon addition of 40 mM Ca(Ac)₂ (FIG. 5A); and interacting in HEPES-glucose buffer (FIG. 5B). Scale bars: 20 μm. White arrows indicate interaction points.

FIG. 6A is a schematic illustration of a bacteria-LUVs fusion with pMPCylated liposomes measured with calcein dequenching assay, as described herein.

FIG. 6B are comparative plots presenting the profile of calcein content mixing assay between bacteria-mimicking membranes and liposomes in the presence and absence of LPS, and calcein-free pMPC-liposomes in the presence and absence of calcium ions (Final concentration: 0.72 mM). Results are an average of 3 independent experiments.

FIGS. 7A-B are representative super-resolution microscopy images of a model Pseudomonas aeruginosa strain (PA14) cells after 4 hours incubation with) pMPCylated-liposomes (FIG. 7A), and PEGylated-liposomes (FIG. 7B), at 37° C. Inserts show a zoomed-in detail of bacteria cells with liposomes adhering to the membrane. Images are presented as overlays of bacteria membrane stained with FM1-43 dye (green) with liposomes labeled with DiR dye (red) visualized using stochastic optical reconstruction microscopy (STORM). Scale bar: 1 μm; insert scale bar: 1 μm.

FIG. 7C is a scatter plot presenting quantification of bacteria cells containing liposomal fluorescence from STORM images, according to FIGS. 7A-B. Each fluorescent locus on the cells' membrane with a minimum size of 180 nm was counted as one individual cell-attached liposomal unit. Differences between groups shown in the box plot were tested with a one-way ANOVA. Boxes represent the 25-75 percentiles of the sample distribution, with black vertical lines representing 1.5×IQR (interquartile range). The black horizontal line represents the median.

FIGS. 8A-B are representative microscopy images of PA14 cells following 4 hour (FIG. 8A) and 24 hour (FIG. 8B) incubation periods, followed by thorough washing, with PEG- (upper panels) and pMPC- (lower panels) calcein-loaded liposomes. Cells were grown at 37° C. for 24 hours prior to the incubation with liposomes. Images are presented as a fluorescent intensity of the calcein signal (green) and overlay between calcein-fluorescence and brightfield. The insert in the lower right panel of FIG. 8A shows a zoomed-in detail of bacteria cells displaying a weak yet recognizable fluorescent intensity. Scale bar: 10 μm; insert scale bar: 1 μm.

FIG. 8C is a scatter plot presenting quantification of single cell microscopy images of the number of cells displaying luminal calcein signal following 24 hours incubation of cells with either PEG-LUVs or pMPC-LUVs, according to FIG. 8B. Differences between groups shown in the box plot were tested with a one-way ANOVA. Boxes represent the 25-75 percentiles of the sample distribution, with black vertical lines representing the 1.5×IQR (interquartile range). The black horizontal line represents the median.

FIGS. 8D-E are kinetic profiles of calcein release from PEG- and pMPC-functionalized liposomes upon interaction with bacterial biofilms of strain PA14 (FIG. 8D) or a model Listeria monocytogenes strain, LESB58 (FIG. 8E), following incubation at 37° C. for a period of 17 hours. Liposomes were either non-functionalized (circles), or functionalized with 5% pMPC (squares) and 5% PEG (triangles) polymer. Calcein-loaded pMPC- LUVs were incubated with naive BM2G media as a negative control (circles). Results are an average of a minimum of 3 experiments.

FIGS. 9A-D are kinetic profiles of calcein release from liposomes functionalized with pMPC- (FIGS. 9A and 9C) or PEG- (FIGS. 9B and 9D) polymer (5%) and extruded through 200 nm (squares), 100 nm (triangles in FIGS. 9A-B and triangles with the apex oriented upwards in FIG. 9C-D) or 50 nm (triangles with the apex oriented downwards in FIGS. 9C-D) pore membrane. The analyses present the effect of liposome diameter and stearylamine presence (FIG. 9A-B) and absence (FIG. 9C-D) on calcein release profiles upon interaction between liposomes and PA14 cells at 37° C. LUVs were extruded through 200 nm pore membrane encapsulating calcein were incubated with BM2G media only as a negative control (LUVs in bacteria-free medium; circles). Results are from a minimum of 3 independent experiments.

FIG. 10 is a schematic illustration of a proposed two-stage mechanism for calcium-mediated adhesion and fusion between exemplary polymeric compound according to some of the present embodiments, pMPC-LUVs, and a bacterial (e.g., Pseudomonas aeruginosa) membrane. Step (1) illustrates an exemplary pMPC-functionalized liposome comprising cholesterol and stearylamine, according to FIG. 1, reaching the bacterial membrane, where the presence of divalent cations (e.g., calcium ions; singular dots) at the cell membrane's surface bridges pMPC with lipopolysaccharides (LPS) and pulls them towards the cell surface; Step (2) illustrates an interaction (e.g., charge-charge interactions) between the liposome and bacterial membrane; and step (3) illustrates a fusion between the liposome and the bacterial membrane.

FIG. 11A is a scatter plot presenting pyocyanin production in P. aeruginosa following treatment with free exemplary antimicrobial agents SMX, EA, SMX/EA, unloaded exemplary pMPC-LUVs, PEG-LUVs- or exemplary pMPC-LUVs- loaded with SMX (SMX:lipid molar ratio of 0.55:1.0) or SMX/EA (SMX:EA:lipid molar ratios 0.22:0.6:1.0). Normalized pyocyanin concentration measured by HPLC following treatment with two subsequent 1 mL doses of 4 hours incubation period each. The data represents minimum 3 biological repeats. Boxes represent the 25-75 percentiles of the sample distribution, with black vertical lines representing the 1.5×IQR (interquartile range). The black horizontal line represents the median.

FIG. 11B is a photograph of the exemplary pMPC-LUVs loaded with SMX/EA.

FIGS. 12A-C are representative cryo-TEM images of pMPCylated vesicles: unloaded liposomes (FIG. 12A), liposomes loaded with SMX (FIG. 12B) and co-loaded with SMX/EA (FIG. 12C). White arrows indicate a dark structure inside liposomes. Scale bar: 50 nm.

FIG. 12D are comparative plots presenting release profiles of SMX, EA, and combined SMX/EA from loaded pMPC-functionalized liposomes at physiological conditions (37° C., pH 7.2), as presented in FIGS. 12A-C. Results are shown as an average and standard deviation of 3 independent experiments. The dashed lines represent a trend of the data points.

FIG. 12E are comparative plots presenting release profiles of SMX and SMX/EA loaded into PEG-functionalized liposomes at physiological condition (37° C., pH 7.2), as presented in FIGS. 13A-C. Results are shown as average and standard deviation of 3 independent experiments. Dashed lines represent trend of the data points.

FIG. 12F is a plot presenting the retention profiles of loaded drugs inside pMPC-liposomes upon storage at 4° C. Results are from a minimum of 2 independent experiments with 3 technical repeats.

FIGS. 13A-C are representative cryo-TEM images of liposomes loaded with SMX (FIG. 13A) and of liposomes co-loaded with SMX/EA (FIGS. 13B-C). Scale bar: 50 nm.

FIG. 14A is a scatter plot presenting PA14 (P. aeruginosa) cell viability following two-dose treatment (4 hours each) of the biofilm with free exemplary antimicrobial agents SMX and SMX/EA, and unloaded or SMX- and SMX/EA- loaded PEG-LUVs or exemplary pMPC-LUVs. The results were quantified by MBEC™-based resazurin assay as described herein. The rightmost boxes (SMX [1.5 mg/mL], SMX/EA [0.7/1.5 mg/mL]) represents treatment with free drugs. Data obtained from 8 or more biological repeats.

FIG. 14B is a scatter plot presenting linear correlation between resazurin assay (I585/I635) as described herein (General bacterial growth conditions) and CFU estimated on growth of PA14 strain.

FIG. 14C is a scatter plot presenting CFU/mL of PA14-biofilm after two-dose treatment with SMX- and SMX/EA- loaded liposomes and free drugs, as described in FIG. 14A. CFU results are calculated from correlation between resazurin assay and CFU according to FIG. 12E. Biofilm was grown on a MBEC™ assay lid, and after two doses of antimicrobial treatment the remaining cells were removed to fresh BM2G by sonication and cell viability was quantified by resazurin assay after 400 minutes incubation period. The rightmost boxes (SMX, SMX/EA) represent treatment with free drugs. Abbreviation: NT-not treated (untreated) biofilm. Differences between groups shown in the box plot were tested with a one-way ANOVA. Boxes represent the 25-75 percentiles of the sample distribution, with black vertical lines representing the 1.5×IQR (interquartile range). The black horizontal line represents the median.

FIG. 15A presents representative confocal microscopy images of PA14-biofilm viability following treatment with drug-loaded LUVs and free drugs on 24 hour-old biofilm evaluated by a LIVE/DEAD™ assay, showing live (green) and dead (red) cells. Images show representative areas from chamber slides. PA14-biofilms were left untreated, or treated with free SMX (1.5 mg/mL), SMX-loaded PEG-LUVs, SMX/EA-loaded PEG-LUVs, SMX/EA-loaded pMPC-LUVs, or SMX-loaded pMPC-LUVs. Scale bar: 50 μm.

FIG. 15B is a scatter plot presenting percentage of dead bacteria quantified from at least four different microscopic images as presented in FIG. 15A. Data represent a minimum of two biological repeats with two technical repeats each.

FIGS. 16A-B are a schematic illustration of the treatment, preparation and examination of a 72 hour-old colony (FIG. 16A), and confocal microscopy images of 10 μm-thin cross-sections of paraffin-embedded PA14-colony following a 4-hour treatment with 5 μL pMPC-LUVs, unloaded or loaded with SMX (FIG. 16B). In FIG. 16B, LIVE/DEAD™ assay was applied prior to fixation, showing live (green) and dead (red) cells; Scale bar: 100 μm.

FIG. 16C are comparative plots presenting spatial profiles for biofilm sections displaying variation in dead bacteria cells fraction upon injection with either SMX-loaded pMPC-LUVs (squares) or PEG-LUVs (triangles), or non-loaded liposomes (circles). Details of the quantification are described in FIGS. 16D-E. Differences between groups shown in box plots were tested with a one-way ANOVA. Boxes represent the 25-75 percentiles of the sample distribution, with black vertical lines representing the 1.5×IQR. The black horizontal line represents the median.

FIG. 16D is a schematic illustration showing the data analysis workflow for quantification of LIVE/DEAD™ signal in images of paraffin-embedded and 10 μm-thin section of colonies, as described in FIG. 16B. Images were contoured to isolate only the biofilm section and remove the fluorescent background signal. Resulting composite images were segmented with a 200 μm wide and 150-200 μm high region of interest.

FIG. 16E is a scatter plot presenting average intensity of the two channels (green/red) for each segment of FIG. 16D. The fraction of dead bacteria was quantified as intensity red channel over the sum of both channels. To compare different biofilm sections, each profile of dead bacteria fraction was normalized as variation from its lowest value.

### DESCRIPTION OF SPECIFIC EMBODIMENTS OF THE INVENTION

The present invention, in some embodiments thereof, relates to therapy, and more particularly, but not exclusively, to drug-loaded liposomal compositions usable, inter alia, for treating (e.g., interfering with a formation of, disrupting and/or eradicating) biofilm in a subject in need thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have studied the effect of drug-loaded functionalized liposomes made of a bilayer-forming lipid and a lipid-polymer conjugate (also referred to herein as LPC, or a polymeric compound) such as described in WO 2017/109784, on treating biofilm, and have tested, during these studies, the effect of various manipulations of the liposome composition. The present inventors have surprisingly uncovered that the inclusion of a positively-charged hydrophobic compound (also referred to herein interchangeably as a positively-charged lipidic agent, as defined herein) in and/or on the liposome membrane, and optionally also a sterol, provides drug-loaded liposomes that are capable, inter alia, of interacting with the biofilm's membrane, and of releasing the drug at the biofilm infection site, and thereby of disrupting and/or eradicating and hence treating the biofilm.

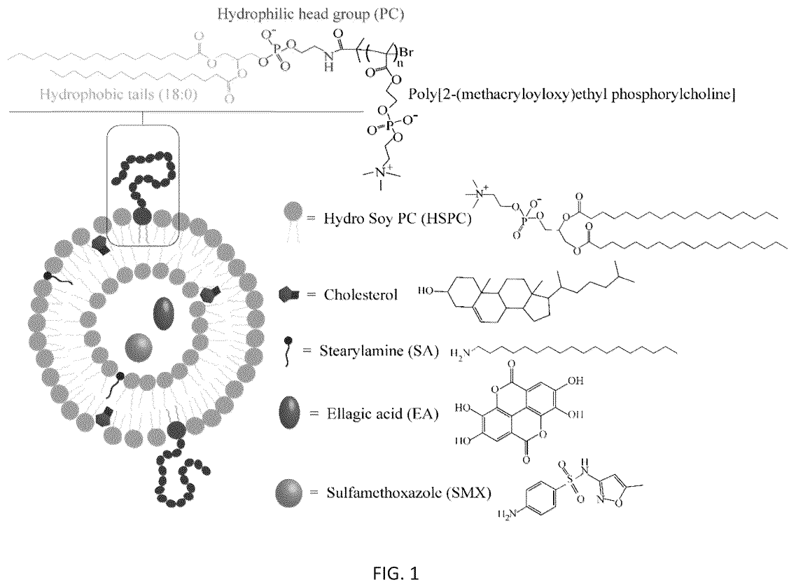

pMPC-functionalized liposomes, as exemplary liposomes containing an LPC as described herein, were used in these studies and were prepared in the presence of an exemplary sterol (cholesterol) and an exemplary positively-charged hydrophobic compound (a cationic lipid such as stearylamine; SA), as shown in FIG. 1. These liposomes were characterized, and their abilities to interact with bacterial cell surface and to permeate biofilms were assessed as means to deliver, e.g., antibacterial agents, thereby eradicate bacterial biofilms.

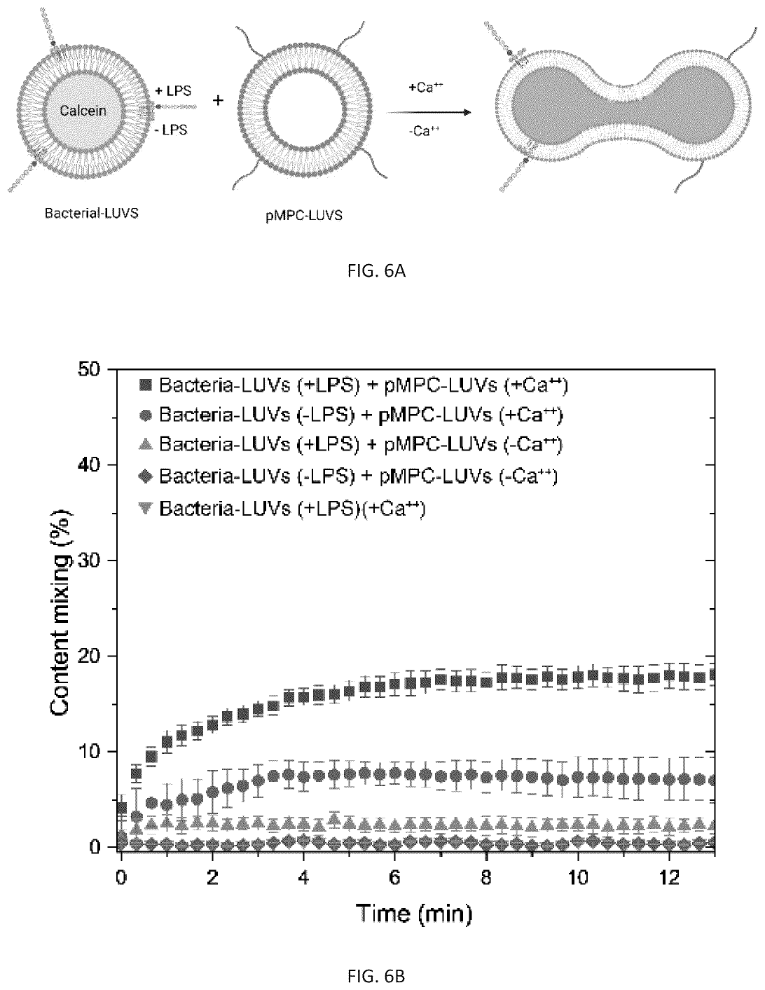

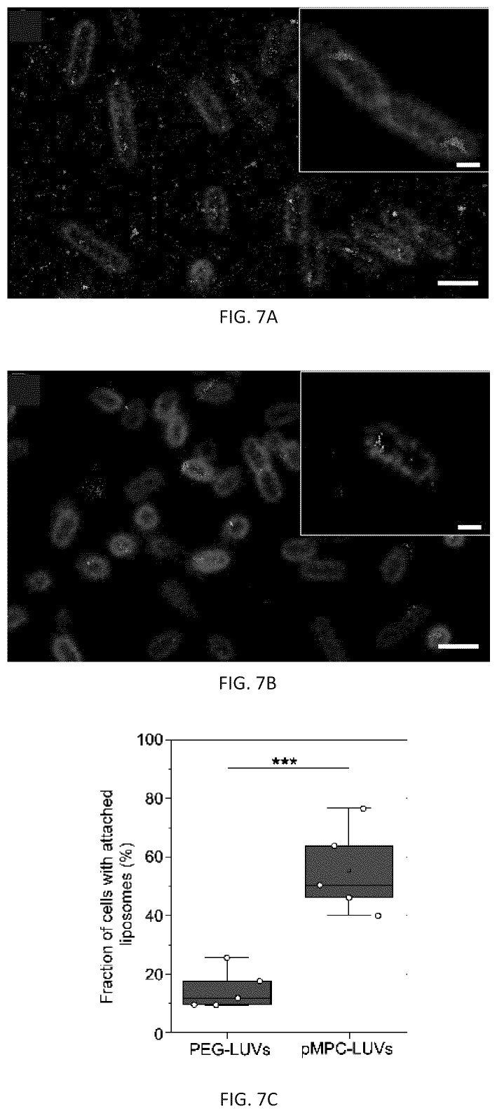

As demonstrated in the Examples section that follows, the pMPC-functionalized liposomal drug delivery vehicles exhibited significantly higher biofilm penetration, cell interaction and biofilm eradication than equivalent free drug or currently used (e.g., PEG-functionalized) liposome-based treatments of such biofilms. See, for example, FIGS. 6B, 7A-C, 8A-E, 9A-D, 11A, 12A-F, 14A, 14C and 15A-B.

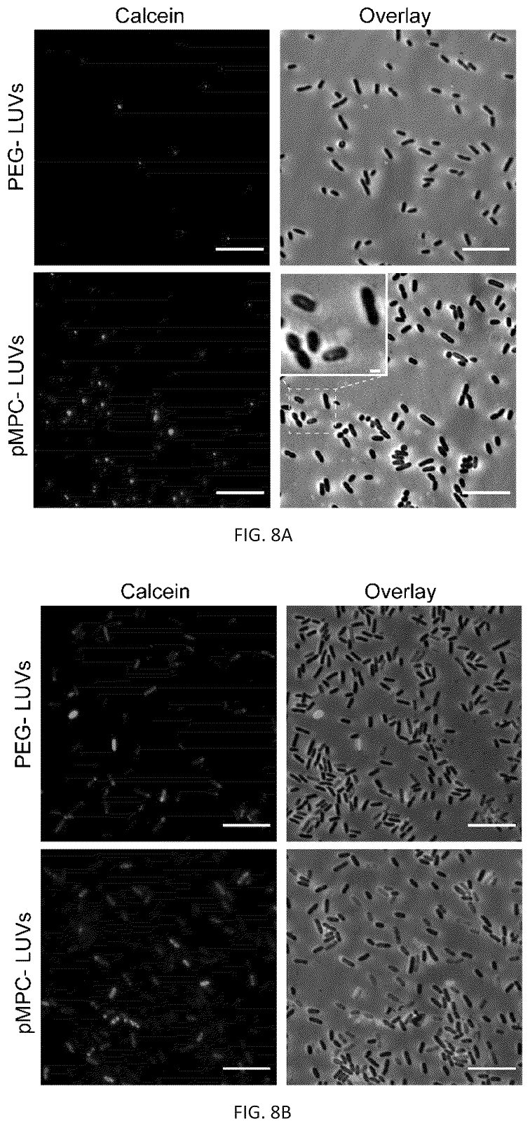

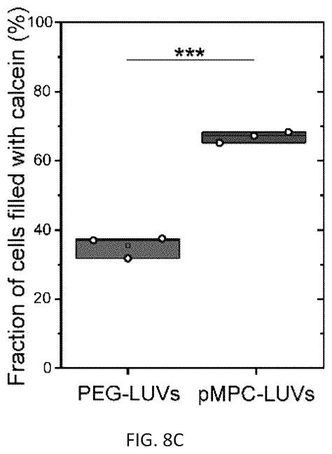

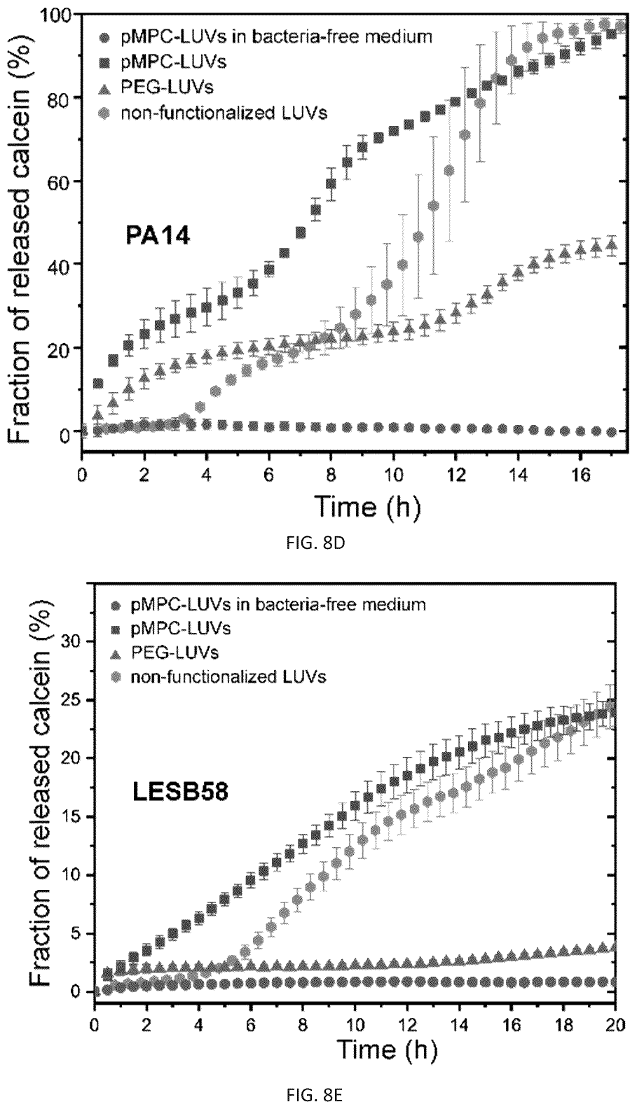

These exemplified liposomes were shown to exhibit targeting towards bacteria cell membranes with 4-fold higher affinity compared to other liposomes (e.g., PEGylated) (see, FIGS. 7A-C) and, as a result of membrane adhesion, these liposomes were shown to fuse with bacteria cells with much higher efficiency (see, FIGS. 8A-C).

It was demonstrated that the observed enhanced fusion results in higher cargo release into the bacteria's cytosol compared to other functionalized counterparts; between 2.5-fold and 5-fold faster for two exemplary bacterial strains, as shown in FIGS. 8D-E, respectively. The enhanced cytosolic release observed for both strains indicates the potential therapeutic effect of the exemplary drug-loaded liposomes against a broad range of biofilm infections.

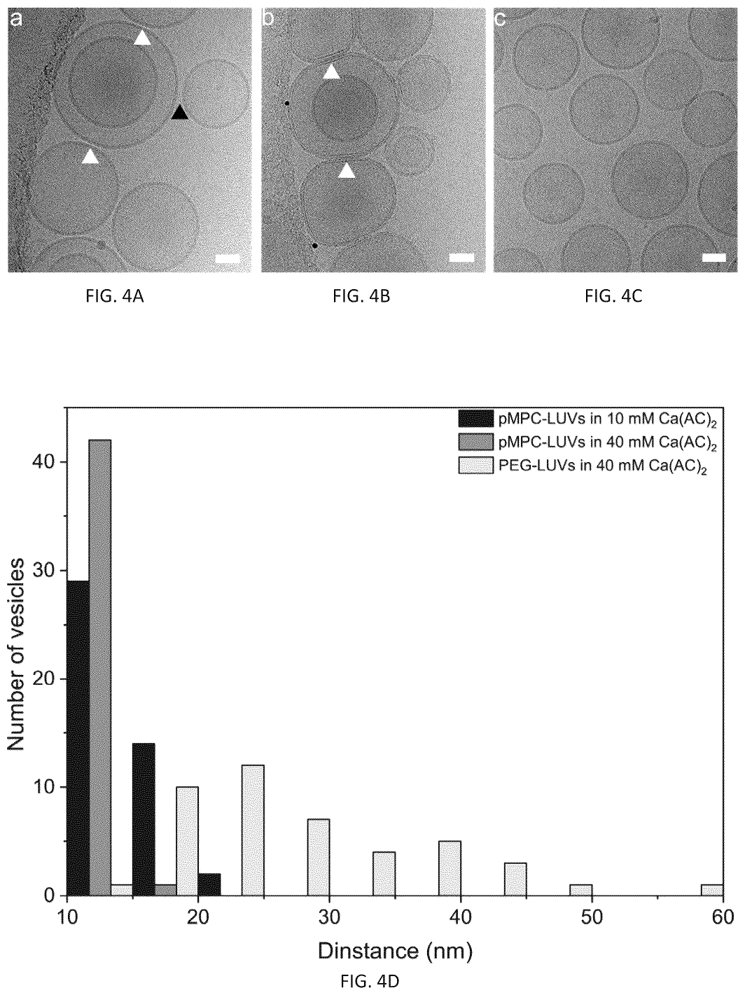

Without being bound to any particular theory, it is assumed that the significant increase in efficiency in the performance of the exemplary pMPCylated liposomes is mediated by the presence of divalent ions, (mainly in the form of Ca⁺²) [Lopez-Laguna et al. Trends Biochem Sci 2020, 45 (11), 992-1003], which leads to a clear attraction between the strong dipole arising from the dual charge nature of the pMPCylated liposomes (see, FIGS. 4A-C and 5A).

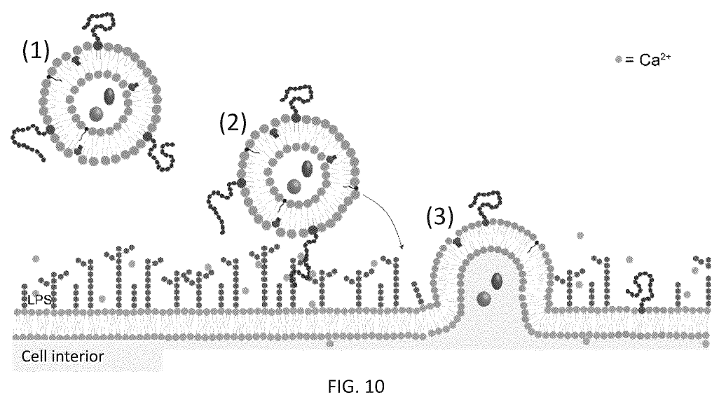

As observed in vitro using calcein dequenching measurement of bacterial-mimicking LUVs (FIG. 6B), the stronger interaction of the exemplary pMPCylated LUVs with the target cells arises from the bridging of the exemplary LPC functionalization to the negatively-charged lipopolysaccharide (LPS) exposed at the cells' surface via divalent cations present at the bacterial membrane [Clifton et al. 2015, supra; and Sun et al. Nat Rev Microbiol 2021, 1-13]. This may be described by a two-stage mechanism as schematically shown in FIG. 10, where LPC (e.g., pMPC) functionalization modulates the liposome-cell adhesion.

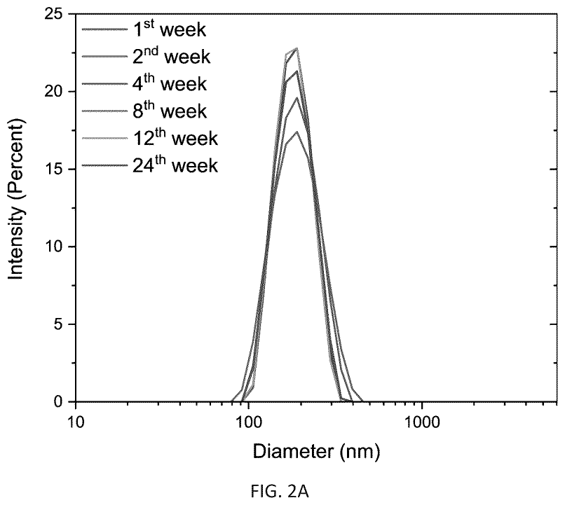

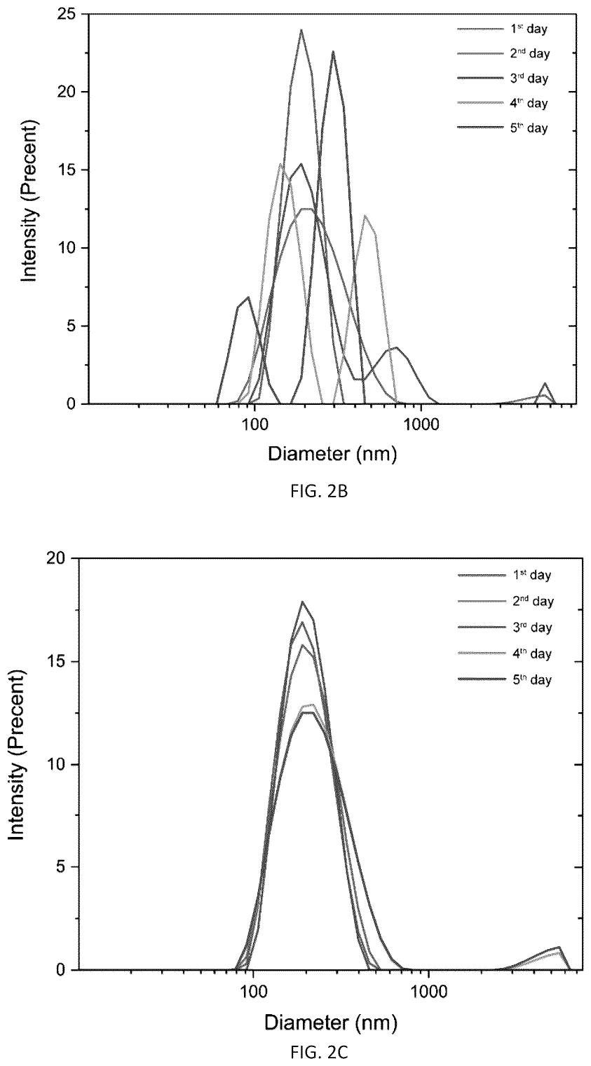

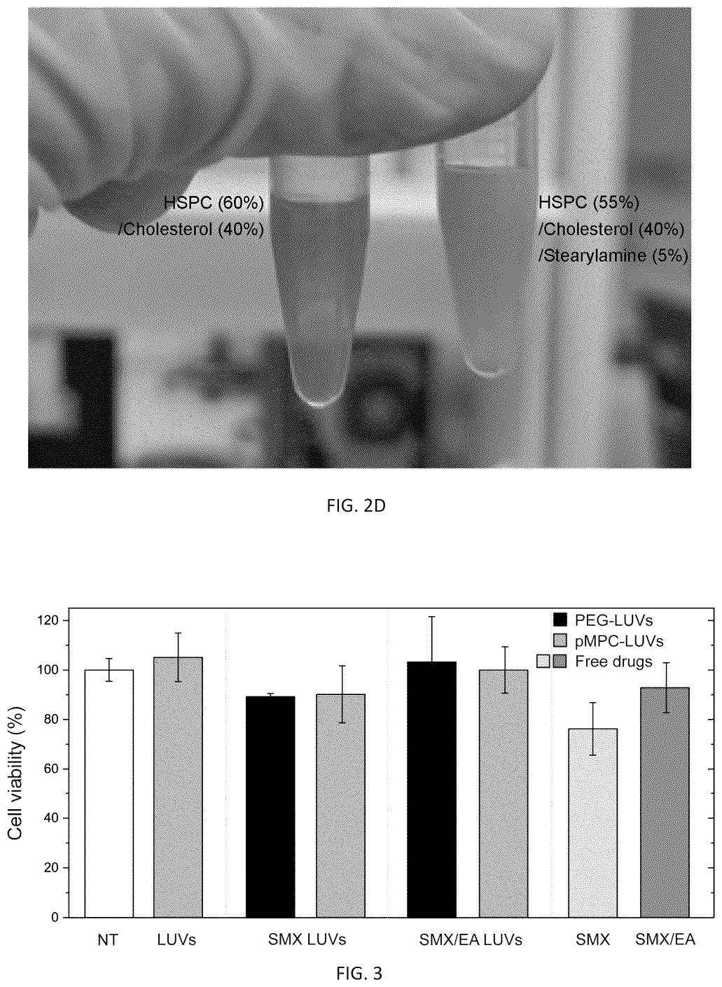

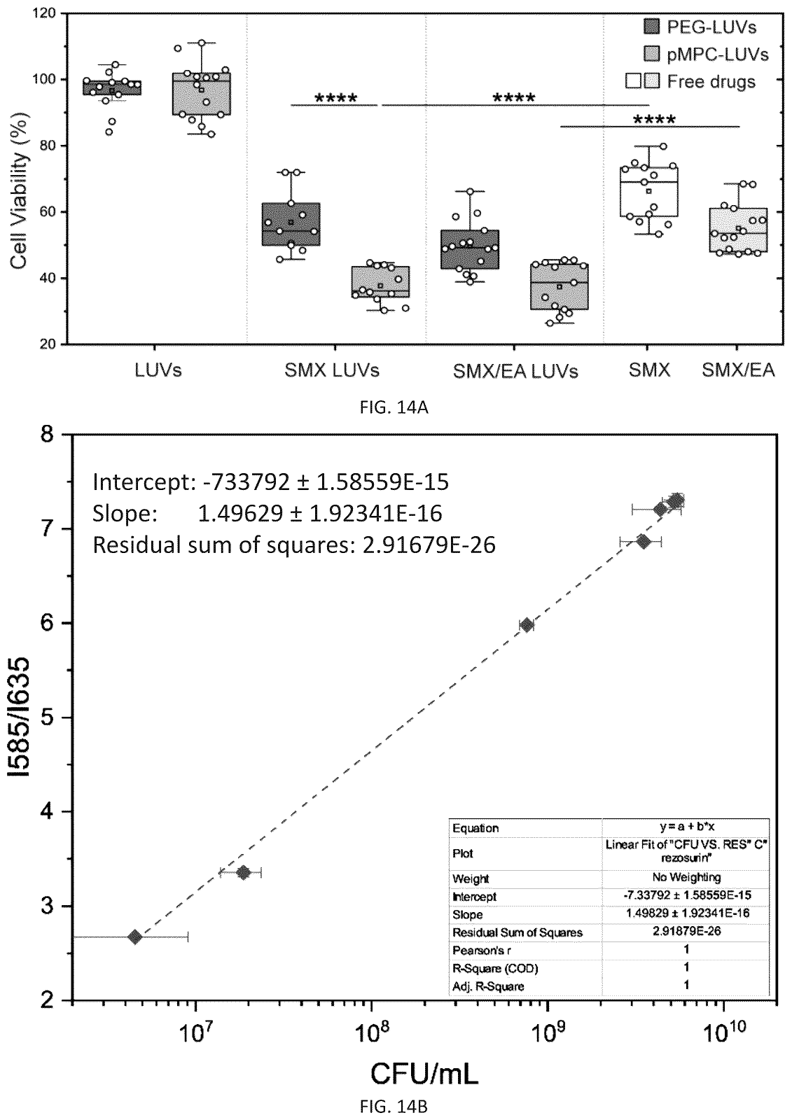

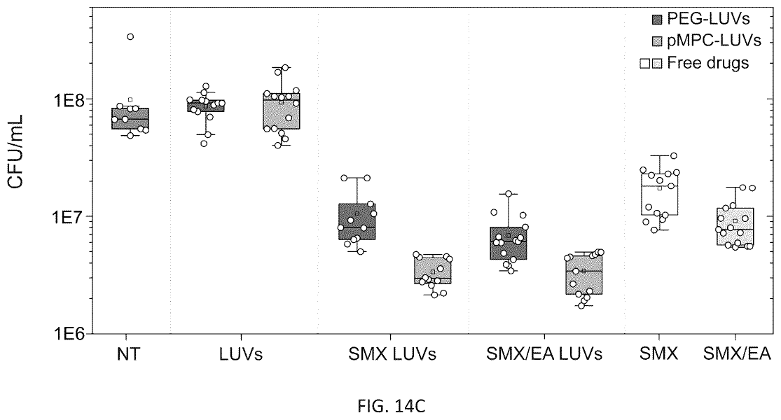

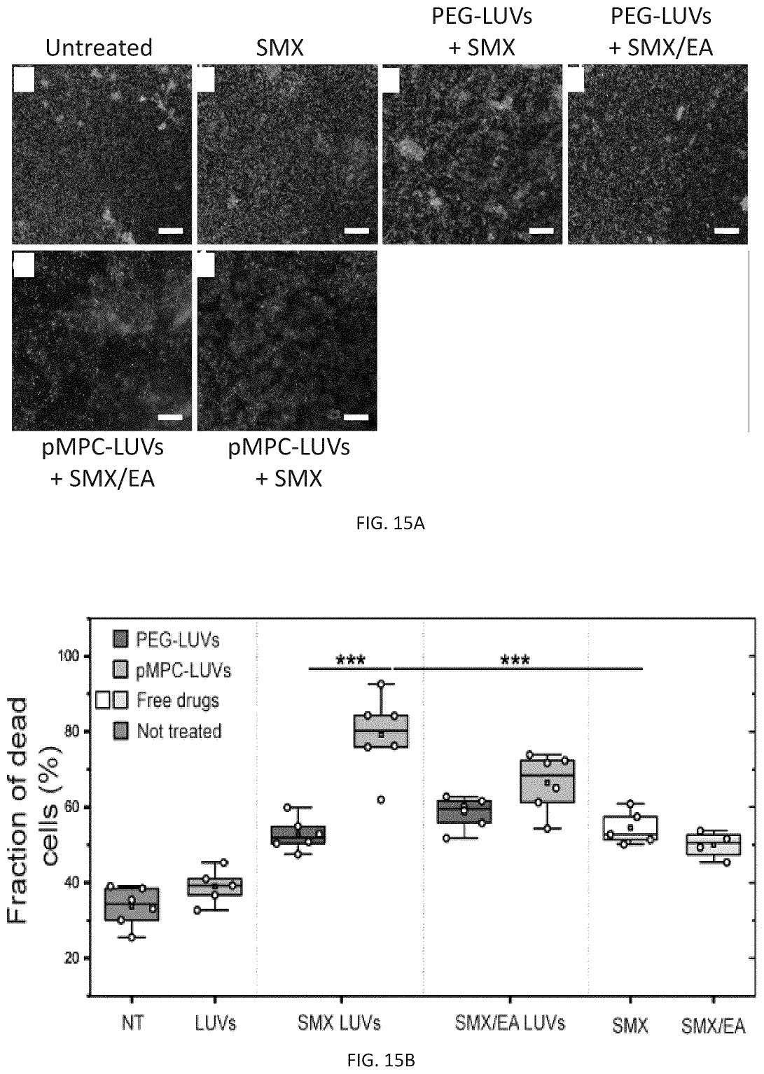

The exemplary drug-loaded pMPCylated liposomes are significantly more efficient in killing target cells in bacterial biofilms, compared to drug-loaded PEG-functionalized liposomal carriers, or to free drugs, as FIG. 15A indicates. Yet, the exemplary pMPCylated vesicles display equivalent biocompatibility (FIG. 14A) and their long-term colloidal stability (FIG. 2A) to those obtained by PEGylated vesicles.

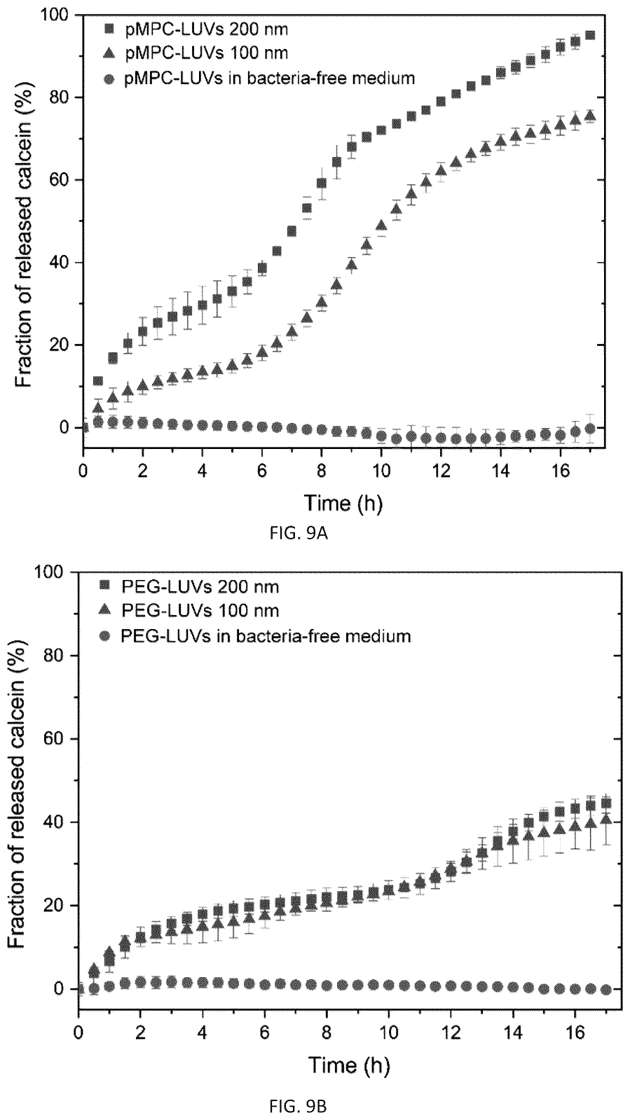

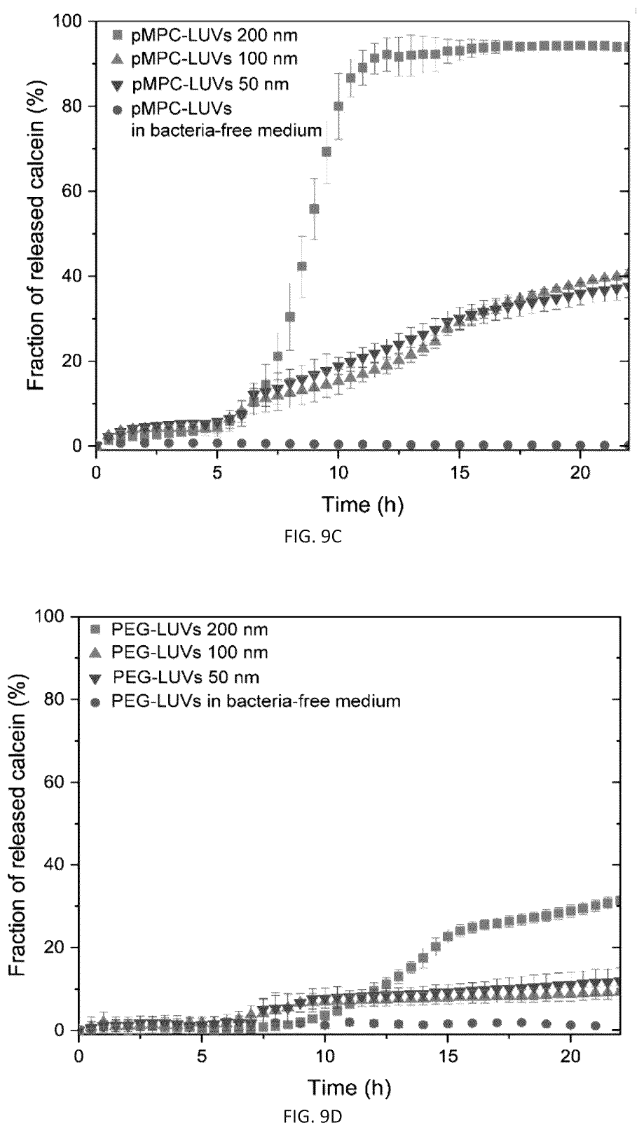

The role of the positively-charged stearyl amine, included in the liposome's membrane, as affecting the interaction with the bacterial biofilms, is demonstrated when comparing FIGS. 9A and 9C.

The drug-loaded exemplary pMPC-liposomes were shown to efficiently and sustainably load single compounds (e.g. the antimicrobial agent SMX) or multiple agents simultaneously (e.g., SMX/ellagic acid). This therapeutic approach relies on multiple drug combinations and results in decreased toxicity of bacterial treatment by employing a lower concentration of antibiotics and enhancing their antimicrobial activity by synergistic interplay (FIGS. 14A-C and 15A-B).

Overall, it has been shown herein that the exemplary functionalized liposomes exhibit enhanced penetration and delivery efficiency to bacterial biofilms, including different strains, and both wet and air-exposed biofilms, as well as multi-drug loadings to improve therapeutic efficacy. Such properties are particularly attractive for applications in clinical settings, where the vast majority of biofilm-based bacterial infections occur.

The enhanced bacterial affinity and penetration of the liposomes as described herein may provide better outcomes for patients (more efficient eradication and corresponding quicker recovery) and lower adversary effects (lower drug dosage).

Embodiments of the present invention relate to newly designed liposomes, formed of a bilayer-forming lipid, a lipid-polymer conjugate, a positively-charged lipidic agent, and optionally a sterol, to such liposomes that have one or more therapeutically active agents associated therewith, as described herein, which are also referred to herein as drug-loaded liposomes, and to the use of these drug-loaded liposomes in the treatment of biofilm (e.g., of biofilm-associated medical conditions).

### Liposomes:

According to an aspect of some embodiments of the invention, there is provided a liposome, the liposome comprising a bilayer-forming lipid and a polymeric compound (referred to herein also as “lipid-polymeric compound” or “LPC”).

In some of any of the embodiments described herein, the liposome further comprises a positively-charged lipidic agent or compound, as described herein.

In some of any of the embodiments described herein, the liposome further comprises a sterol.

In some of any of the embodiments described herein, the liposome further comprises a positively-charged lipidic agent or compound and a sterol.

According to the present embodiments, the liposome comprises one or more therapeutically active agent(s), as described in further detail hereinunder.

According to some of any of the embodiments described herein, the liposome is characterized by a diameter (e.g., an average diameter) in a range of from 10 nm to 1000 nm, or from 10 nm to 500 nm, or from 50 nm to 500 nm, or from 10 nm to 300 nm, or from 50 nm to 300 nm, or from 10 nm to 200 nm, or from 50 nm to 250 nm, or from 50 nm to 200 nm, or from 100 nm to 200 nm, including any intermediate values and subranges therebetween. In some of any of the embodiments described herein, the liposome is characterized by a diameter (e.g., an average diameter) higher than 100 nm, for example, of from 100 nm to 200 nm.

In some of any of the embodiments described herein, the liposome is characterized by a diameter (an average diameter) of 100 nm or lower, for example, in the range of from 10 nm to 100 nm, or from 50 nm to 100 nm, including any intermediate values and subranges therebetween.

A liposome according to any of the embodiments described herein, or according to combination thereof, may be approximately spherical in shape or may have any alternative shape, such as an elongated tube and/or a flattened (e.g., sheet-like) shape.

### Bilayer-Forming Lipid:

The liposome as described herein in any of the embodiments comprises at least one bilayer forming lipid.

Herein, the term “bilayer-forming lipid” encompasses any compound in which a bilayer may form from a pure aqueous solution of the compound. The formed bilayer comprises two parallel layers of lipid molecules.

In some embodiments of any one of the embodiments described herein, the bilayer-forming lipid comprises at least one charged group (e.g., one or more negatively charged groups and/or one or more positively charged groups).

In some embodiments, the bilayer-forming lipid is zwitterionic; comprising both (e.g., an equal number of) negatively charged and positively charged groups (e.g., one of each).

In some embodiments of any of the embodiments described herein, a polymeric moiety in comprises a lipid moiety represented by the variable X in formula I (according to any of the respective embodiments described herein) which comprises a residue of a bilayer-forming lipid (e.g., a glycerophospholipid) which is comprised by or which is closely related to the bilayer-forming lipid, for example, wherein the lipid moiety comprises a glycerophospholipid residue and the liposome comprises another glycerophospholipid as a bilayer-forming lipid (e.g., optionally wherein fatty acid residues in the glycerophospholipid residue have about the same length as fatty acid residues in the bilayer-forming lipid, and optionally wherein the fatty acid residues in the glycerophospholipid residue are substantially the same as the fatty acid residues in the bilayer-forming lipid).

Examples of bilayer-forming lipids are described in WO 2017/109784, and include, but are not limited to, fatty acids, monoglycerides, diglycerides, triglycerides, phospholipids, glycerophospholipids, sphingolipids, and sterol.

In exemplary embodiment, the bilayer-forming lipid is a phospholipid, for example, a glycerophosphlipid.

In some embodiments of any of the embodiments described herein, a molar ratio of the bilayer-forming lipid and the polymeric compound in the liposome is in a range of from 5:1 to 5,000:1 (bilayer-forming lipid: polymeric compound), optionally in a range of from 10:1 to 2,500:1, optionally in a range of from 25:1 to 1,000:1, and optionally in a range of from 50:1 to 500:1, including any intermediate values and subranges therebetween, by % mol.

### Positively-Charged Lipidic Compound:

According to some of any of the embodiments described herein, the positively-charged lipidic agent or compound is incorporated within a lipid bilayer and/or on a surface of the liposome as described in any of the embodiments.

Herein, the terms “lipidic agent” or “lipidic compound” or “lipidic substance” are used interchangeably and describe a chemical or biochemical agent (e.g., compound, substance) that is composed of a lipid and/or is used in the biochemical and/or synthetic formation or modification of lipids, and/or is able to interact with lipids, e.g., phospholipids, via, for example, hydrophobic interactions. In some embodiments, the lipidic agent comprises at least one hydrophobic moiety that is derived from a lipid, for example, from a fatty acid, a monoglyceride, a diglyceride, a triglyceride, a glycerophospholipid, a sphingolipid, and/or a sterol.

The positively-charged lipidic agent according to the present embodiments comprises a lipid moiety, derived from a lipid, as described herein, and one or more moieties or groups that are positively charged at physiological conditions (e.g., at pH 6-8). Such moieties are or comprise one or more of an amine, as defined herein, including primary, secondary and tertiary, aromatic, aliphatic and alicyclic amines, an imine, a hydrazine, a guanidine, etc.

In some of any of the embodiments described herein, the positively-charged lipidic agent comprises a hydrocarbon chain of from 4 to 30, or from 8 to 26, or from 12 to 22, carbon atoms in length, substituted and/or terminated by at least one substituent that is positively charged at physiological conditions.

Herein, the term “positively-charged” refers to an agent (e.g., molecule) that has an overall positive charge in physiological conditions, i.e., at pH of 6-8 and at a temperature of about 37° C.

In some of any of the embodiments described herein, the positively-charged lipidic agent is substituted and/or terminated by one or more amines, as defined herein.

In some of any of the embodiments described herein, the positively-charged lipidic agent comprises a hydrocarbon chain of from 4 to 30, or from 8 to 26, or from 12 to 22, carbon atoms in length, substituted and/or terminated by one or more amines.

In some of any of the embodiments described herein, the positively-charged lipidic agent or compound comprises a moiety derived from a fatty acid, for example, from lauric acid, myristic acid, palmitic acid, stearic acid, palmitolic acid, oleoic acid, or linoleic acid, which is substituted and/or terminated by one or more amines.

In some of any of the embodiments described herein, the positively-charged lipidic agent or compound comprises a moiety derived from a fatty acid, for example, from lauric acid, myristic acid, palmitic acid, stearic acid, palmitolic acid, oleoic acid, or linoleic acid, in which the terminal carboxylic acid is replaced by amine, and can be, for example, lauryl amine, myristyl amine, palmityl amine, stearyl amine, palmitolyl amine, oleyl amine, or linolyl amine.

In exemplary embodiments, the positively-charged lipidic agent is stearyl amine.

In some of any of the embodiments described herein, a liposome comprising the positively-charged lipidic agent or compound is characterized by an increased colloidal stability in comparison with the same liposome without the positively-charged lipidic agent. The determination and/or assessment of colloidal stability of a liposome is known in the art, e.g., kinetic measurements using dynamic light scattering (DLS).

In some of any of the embodiments described herein, a liposome comprising the positively-charged lipidic agent or compound is characterized by an increased affinity towards biofilm in comparison with the same liposome without the positively-charged lipidic agent. The determination and/or assessment of a liposome's affinity towards biofilm in the present context is known in the art, e.g., fluorescence microscopy, or calcein release as described herein.

In some embodiments of any of the embodiments described herein, a molar ratio of the bilayer-forming lipid and the positively-charged lipidic agent is in a range of from 1:1 to 100:1, or from 1:1 to 50:1 (bilayer-forming lipid: positively-charged lipidic agent), including any intermediate values and subranges therebetween, by % mol. In some embodiments, a molar ratio of the bilayer-forming lipid and the positively-charged lipidic agent is about 20:1 (bilayer-forming lipid: positively-charged lipidic agent), by % mol.

In some embodiments of any of the embodiments described herein, a molar ratio of the positively-charged lipidic agent and the polymeric compound is in a range of from 100:1 to 1:100, or from 10:1 to 1:10 (positively-charged lipidic agent: polymeric compound), including any intermediate values and subranges therebetween, by % mol. In some embodiments, a molar ratio of the positively-charged lipidic agent and the polymeric compound is about 1:1 (positively-charged lipidic agent: polymeric compound), by % mol.

### Sterol:

According to some of any of the embodiments described herein, the sterol is bound to a surface of the liposome and/or within a lipid bilayer and/or core of the liposome, as described in any of the embodiments.

In some of any of the embodiments described herein, or in any combination thereof, the sterol is cholesterol. In some of any of the embodiments described herein, a molar ratio of the bilayer-forming lipid and the sterol is in a range of from 10:1 to 1:10, or from 5:1 to 1:10, or from 10:1 to 5:1 (bilayer-forming lipid: sterol), including any intermediate values and subranges therebetween, by % mol. In some of any of the embodiments described herein, a molar ratio of the bilayer-forming lipid and the sterol is about 5:2, (bilayer-forming lipid: sterol), by % mol.

In some of any of the embodiments described herein, the sterol provides the liposome with an increased flexibility and/or a decrease rigidity, compared to the same liposome without the presence of the sterol.

### Polymeric Compound:

Herein, the term “polymeric” refers a compound having at least 2 repeating units (and more preferably at least 3 repeating units), the repeating units being identical or similar. It is to be appreciated that the compound of general formula I is by definition polymeric when n is at least 2, as it comprises at least 2 of the backbone units represented by Y.

The polymeric compound which forms a liposome as described herein, comprises a polymeric moiety conjugated to a lipid moiety, and is also referred to herein as a lipid polymeric conjugate, or LPC.

The polymeric compound according to the present embodiments is represented by the general formula I:

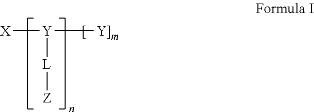

wherein:
m is zero or a positive integer;
n is an integer which is at least 1;
X is a lipid moiety, wherein when X does not comprise a phosphate group, n is at least 2;
Y is a backbone unit which forms a polymeric backbone;
L is absent or is a linking moiety; and
Z has the general formula II:

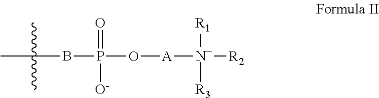

wherein:
A is a substituted or unsubstituted hydrocarbon;
B is an oxygen atom or is absent; and
R₁-R₃ are each independently hydrogen, alkyl, cycloalkyl, heteroalicyclic, aryl or heteroaryl,
as described in more detail herein below.
Formula I may also be described herein simply as:

X—[Y(-L-Z)]n[Y]m
which is to be regarded as interchangeable with the schematic depiction hereinabove.

Herein, the phrase “polymeric moiety” refers to the portion of the polymeric compound (according to any of the embodiments described herein relating to general formula I) which has the general formula Ia:

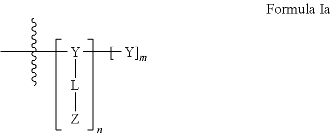

wherein m, n, Y, L and Z are as defined herein for general formula I.

Formula Ta may also be described herein simply as:

—[Y(-L-Z)]n[Y]m
which is to be regarded as interchangeable with the schematic depiction hereinabove.

Herein, the phrase “polymeric compound” further encompasses compounds having a “polymeric moiety” as described herein having one unit (e.g., according to formula Ia wherein n is 1), provided that the lipid moiety described herein (e.g., the lipid moiety represented by X) has a similar unit. For example, when the lipid moiety comprises a phosphate group (e.g., the lipid moiety is a glycerophospholipid moiety), such that the lipid moiety has a phosphate group and a single unit of the polymeric moiety has a phosphate group, the two phosphate groups may be regarded as repeating units.

In preferred embodiments however, n is at least 2, such that the polymeric moiety per se has at least two units. In some embodiments, n is at least 3.

As used herein, the term “backbone unit” refers to a repeating unit, wherein linkage of a plurality of the repeating unit (e.g., sequential linkage) forms a polymeric backbone. A plurality of linked repeating units per se is also referred to herein as a “polymeric backbone”.

As shown in formulas I and Ia, L and Z together form a pendant group of at least a portion of the backbone units, which group is referred to herein for brevity simply as the “pendant group”.

Each backbone unit Y with pendant group (i.e., a unit represented by Y(-L-Z), the number of which is represented by the variable n) and each backbone unit Y without a pendant group (the number of which is represented by the variable m) is also referred to herein as a “monomeric unit”.

A backbone unit may optionally be a residue of a polymerizable monomer or polymerizable moiety of a monomer. A wide variety of polymerizable monomers and moieties will be known to the skilled person, and the structure of the residues of such monomers which result upon polymerization (e.g., monomeric units) will also be known to the skilled person.

A “residue of a polymerizable monomer” refers to a modified form of a polymerizable monomer and/or a portion of a polymerizable monomer that remains after polymerization.

A portion of a polymerizable monomer may be formed, for example, by a condensation reaction, e.g., wherein at least one atom or group (e.g., a hydrogen atom or hydroxyl group) in the monomer, and optionally at least two atoms or groups (e.g., a hydrogen atom and a hydroxyl group) in the monomer, is replaced with a covalent bond with another polymerizable monomer.

A modified form of a polymerizable monomer may be formed, for example, by ring-opening (wherein a covalent bond between two atoms in a ring is broken, and the two atoms optionally each become linked to another polymerizable monomer); and/or by adding to an unsaturated bond, wherein an unsaturated bond between two adjacent atoms is broken (e.g., conversion of an unsaturated double bond to a saturated bond, or conversion of an unsaturated triple bond to an unsaturated double bond) and the two atoms optionally each become linked to another polymerizable monomer.

A modified form of a polymerizable monomer may consist essentially of the same atoms as the original monomer, for example, different merely in the rearrangement of covalent bonds, or alternatively, may have a different atomic composition, for example, wherein polymerization includes a condensation reaction (e.g., as described herein).

Examples of backbone units include, without limitation, substituted or unsubstituted hydrocarbons (which may form a substituted or unsubstituted hydrocarbon backbone), such as alkylene units; hydroxycarboxylic acid units (which may form a polyester backbone), e.g., glycolate, lactate, hydroxybutyrate, hydroxyvalerate, hydroxycaproate and hydroxybenzoate units; dicarboxylic acid units (which may form a polyester backbone in combination with a diol and/or a polyamide in combination with a diamine), e.g., adipate, succinate, terephthalate and naphthalene dicarboxylic acid units; diol units (which may form a polyether backbone, or form a polyester backbone in combination with a dicarboxylic acid), e.g., ethylene glycol, 1,2-propanediol, 1,3-propanediol, 1,4-butanediol, and bisphenol A units; diamine units (which may form a polyamide backbone in combination with a dicarboxylic acid), e.g., para-phenylene diamine and alkylene diamines such hexylene diamine; carbamate units (which may form a polyurethane backbone); amino acid residues (which may form a polypeptide backbone); and saccharide residues (which may form a polysaccharide backbone).

In some embodiments of any of the embodiments described herein, Y is a substituted or unsubstituted alkylene unit.

In some embodiments, Y is a substituted or unsubstituted ethylene unit, that is, an alkylene unit 2 atoms in length.

Polymeric backbones wherein Y is a substituted or unsubstituted ethylene unit may optionally be a polymeric backbone such as formed by polymerizing ethylene (CH₂═CH₂) and/or substituted derivatives thereof (also referred to herein as “vinyl monomers”). Such polymerization is a very well-studied procedure, and one of ordinary skill in the art will be aware of numerous techniques for effecting such polymerization.

It is to be understood that any embodiments described herein relating to a polymeric backbone formed by a polymerization encompass any polymeric backbone having a structure which can be formed by such polymerization, regardless of whether the polymeric backbone was formed in practice by such polymerization (or any other type of polymerization).

As is well known in the art, the unsaturated bond of ethylene and substituted ethylene derivatives becomes saturated upon polymerization, such that the backbone units in a polymeric backbone are saturated, although they may be referred to as units of an unsaturated compound (e.g., a “vinyl monomer” or “olefin monomer”) to which they are analogous.

Polymers which can be formed from unsaturated monomers such as vinyl monomers and olefin monomers are also referred to by the terms “polyvinyl” and “polyolefin”.

Herein, an “unsubstituted” alkylene unit (e.g., ethylene unit) refers to an alkylene unit which does not have any substituent other than the pendant group discussed herein (represented as (-L-Z)). That is, an alkylene unit attached to the aforementioned pendant group is considered unsubstituted if there are no substituents at any other positions on the alkylene unit.

In some embodiments of any of the embodiments described herein, Y has the formula —CR₄R₅—CR₆D—.

When Y is a backbone unit which is not attached to L or Z (i.e., to a pendant group described herein), D is R₇ (an end group, as defined herein); and when Y is a backbone unit which is attached to L or Z, D is a covalent bond or a linking group attaching Y to L or Z. The linking group may optionally be —O—, —S—, arylene, sulfinyl, sulfonyl, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, or amino.

R₄-R₇ are each independently hydrogen, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azide, azo, phosphate phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, or amino.

Herein, the phrase “linking group” describes a group (e.g., a substituent) that is attached to two or more moieties in the compound.

Herein, the phrase “end group” describes a group (e.g., a substituent) that is attached to a single moiety in the compound via one atom thereof.

When each of R₄-R₆ is hydrogen, and D is a covalent bond or linking group, Y is an unsubstituted ethylene group attached (via D) to a pendant group described herein.

When each of R₄-R₇ is hydrogen (and D is R₇), Y is an unsubstituted ethylene group which is not attached to a pendant group described herein.

In some embodiments of any of the embodiments described herein, R₄ and R₅ are each hydrogen. Such embodiments include polymeric backbones formed from many widely used vinyl monomers (including ethylene), including, for example, olefins (e.g., ethylene, propylene, 1-butylene, isobutylene, 4-methyl-1-pentene), vinyl chloride, styrene, vinyl acetate, acrylonitrile, acrylate and derivatives thereof (e.g., acrylate esters, acrylamides), and methacrylate and derivatives thereof (e.g., methacrylate esters, methacrylamides).

In some embodiments of any of the embodiments described herein, R₆ is hydrogen. In some such embodiments, R₄ and R₅ are each hydrogen.

In some embodiments of any of the embodiments described herein, R₆ is methyl. In some such embodiments, R₄ and R₅ are each hydrogen. In some such embodiments, the backbone unit is a unit of methacrylate or a derivative thereof (e.g., methacrylate ester, methacrylamide).

In some embodiments of any of the embodiments described herein, the linking group represented by the variable D is —O—, —C(═O)O—, —C(═O)NH— or phenylene. In exemplary embodiments, D is —C(═O)O—.

For example, the backbone unit may optionally be a vinyl alcohol derivative (e.g., an ester or ether of a vinyl alcohol unit) when D is —O—; an acrylate or methacrylate derivative (e.g., an ester of an acrylate or methacrylate unit) when D is —C(═O)O—; an acrylamide or methacrylamide unit when D is —C(═O)NH—; and/or a styrene derivative (e.g., a substituted styrene unit) when D is phenylene.

In some embodiments of any of the embodiments described herein, L is a substituted or unsubstituted hydrocarbon from 1 to 10 carbon atoms in length. In some embodiments, the hydrocarbon is unsubstituted. In some embodiments, the hydrocarbon is a linear, unsubstituted hydrocarbon, that is, —(CH₂)i— wherein i is an integer from 1 to 10.

In some embodiments of any of the embodiments described herein, L is a substituted or unsubstituted ethylene group. In some embodiments, L is an unsubstituted ethylene group (—CH₂CH₂—).

In some embodiments of any of the embodiments described herein, B is an oxygen atom. In some such embodiments, L is a hydrocarbon according to any of the respective embodiments described herein (i.e., L is not absent), and Z is a phosphate group attached to L.

In some embodiments of any of the embodiments described herein, B is absent. In some such embodiments, L is a hydrocarbon according to any of the respective embodiments described herein (i.e., L is not absent), and Z is a phosphonate group attached to L. In some embodiments, L is also absent, such that the phosphorus atom of formula II is attached directly to Y.

In some embodiments of any of the embodiments described herein, A is a substituted or unsubstituted hydrocarbon from 1 to 4 carbon atoms in length.

In some embodiments of any of the embodiments described herein, A is an unsubstituted hydrocarbon. In some such embodiments, the unsubstituted hydrocarbon is from 1 to 4 carbon atoms in length. In some embodiments, the hydrocarbon is a linear, unsubstituted hydrocarbon, that is, —(CH₂)ⱼ—wherein j is an integer from 1 to 4.

In some embodiments of any of the embodiments described herein, A is a substituted or unsubstituted ethylene group.

In some embodiments of any of the embodiments described herein, A is an unsubstituted ethylene group (—CH₂CH₂—). In such embodiments, the moiety having general formula II (represented by the variable Z) is similar or identical to a phosphoethanolamine or phosphocholine moiety. Phosphoethanolamine and phosphocholine moieties are present in many naturally occurring compounds (e.g., phosphatidylcholines, phosphatidylethanolamines).

In some embodiments of any of the embodiments described herein, A is an ethylene group substituted by a C-carboxy group. In some embodiments, the C-carboxy is attached to the carbon atom adjacent to the nitrogen atom depicted in formula II (rather than the carbon atom attached to the depicted oxygen atom). In such embodiments, the moiety having general formula II (represented by the variable Z) is similar or identical to a phosphoserine moiety. Phosphoserine is present in many naturally occurring compounds (e.g., phosphatidylserines).

Without being bound by any particular theory, it is believed that moieties similar or identical to naturally occurring moieties such as phosphocholine, phosphoethanolamine and/or phosphoserine may be particularly biocompatible.

In some embodiments of any of the embodiments described herein, R₁-R₃ (the substituents of the nitrogen atom depicted in general formula II) are each independently hydrogen or C₁₋₄-alkyl. In some embodiments, R₁-R₃ are each independently hydrogen or methyl. In some embodiments, R₁-R₃ are each methyl. In some such embodiments, R₁-R₃ are each hydrogen.

The variable n may be regarded as representing a number of backbone units (represented by the variable Y) which are substituted by the pendant group represented by (-L-Z), and the variable m may be regarded as representing a number of backbone units which are not substituted by such a pendant group. The sum n+m may be regarded as representing the total number of backbone units in the polymeric backbone. The ratio n/(n+m) may be regarded as representing the fraction of backbone units which are substituted by the pendant group represented by (-L-Z).

The backbone unit Y substituted by the pendant group may be the same as or different than the backbone unit Y which is not substituted by the pendant group (e.g., when m is at least 1).

The plurality (indicated by the variable n) of backbone units Y substituted by the pendant group may be the same as each other or different from each other.

In addition, the plurality (indicated by the variable n) of pendant groups (-L-Z) attached to a plurality of backbone units Y may be the same as each other or different from each other (e.g., may differ in the identity of any one or more of A, B, R₁, R₂, R₃ and L).

In any of the embodiments described herein wherein more than one backbone unit Y is not substituted by the pendant group described herein (i.e., when m is more than 1), the plurality (indicated by the variable m) of backbone units Y which are substituted by the pendant group may be the same as each other or different from each other.

The number of types of backbone units substituted by the pendant group, the number of types of backbone units not substituted by the pendant group (if any such units are present), and/or the number of types of pendant group in the polymeric moiety may each independently be any number (e.g., 1, 2, 3, 4, 5, 6, 7, 8, 9, 10 or more).

In some embodiments of any of the embodiments described herein, the polymeric moiety is a copolymer moiety, that is, the polymeric moiety comprises at least two different types of monomeric unit. The different types of monomeric unit may differ in whether they comprise the pendant group (-L-Z) according to any of the respective embodiments described herein (e.g., when m is at least 1), differ in the type of backbone unit Y, and/or differ in the type of pendant group (-L-Z).

For example, in some embodiments of any of the embodiments described herein the backbone unit Y in each of the Y(-L-Z) units may optionally be the same or different, while the L and Z moieties are the same among the Y(-L-Z) units. In some such embodiments, backbone units not substituted by the pendant group (if any such units are present) may optionally be the same as backbone unit Y in each of the Y(-L-Z) units. Alternatively, backbone units not substituted by the pendant group (if any such units are present) may optionally be different than backbone unit Y in each of the Y(-L-Z) units (while optionally being the same among all backbone units not substituted by the pendant group).

In some embodiments of any of the embodiments described herein the L moiety in each of the Y(-L-Z) units may optionally be the same or different, while the backbone units Y and the Z moieties are the same among the Y(-L-Z) units. In some such embodiments, backbone units not substituted by the pendant group (if any such units are present) may optionally be the same as backbone unit Y in each of the Y(-L-Z) units. Alternatively, backbone units not substituted by the pendant group (if any such units are present) may optionally be different than backbone unit Y in each of the Y(-L-Z) units (while optionally being the same among all backbone units not substituted by the pendant group).

In some embodiments of any of the embodiments described herein the Z moiety in each of the Y(-L-Z) units may optionally be the same or different, while the backbone units Y and the Z moieties are the same among the Y(-L-Z) units. In some such embodiments, backbone units not substituted by the pendant group (if any such units are present) may optionally be the same as backbone unit Y in each of the Y(-L-Z) units. Alternatively, backbone units not substituted by the pendant group (if any such units are present) may optionally be different than backbone unit Y in each of the Y(-L-Z) units (while optionally being the same among all backbone units not substituted by the pendant group).

In any of the embodiments described herein wherein the polymeric moiety is a copolymer moiety, any two or more different types of monomeric unit may be distributed randomly or non-randomly throughout the polymeric moiety. When different types of monomeric unit are distributed non-randomly, the copolymer may be one characterized by any non-random distribution, for example, an alternating copolymer, a periodic copolymer, and/or a block copolymer.

In some embodiments of any of the embodiments described herein, at least a portion of the monomeric units of the polymeric moiety comprise a targeting moiety (according to any of the embodiments described herein relating to a targeting moiety).

A targeting moiety may optionally be comprised by a backbone unit Y according to any of the respective embodiments described herein, linking moiety L according to any of the respective embodiments described herein, and/or moiety Z according to any of the respective embodiments described herein, for example, wherein a substituent according to any of the respective embodiments described herein comprises (and optionally consists of) the targeting moiety. For example, in some embodiments wherein at least a portion of backbone units Y have the formula —CR₄R₅—CR₆D—(as described herein in any of the respective embodiments), any one or more of R₄-R₆ and D (optionally wherein D is R₇ as described herein) comprises a targeting moiety according to any of the respective embodiments described herein (e.g., wherein any one or more of R₄-R₆ and D is a substituted group, comprising a substituent which is a targeting moiety), and optionally any one or more R₄-R₆ and D is a targeting moiety. However, many other structures of monomeric units comprising a substituent which comprises (and optionally consist of) a targeting moiety are also encompassed by embodiments of the invention.

When Y is a backbone unit which is not attached to L or Z (i.e., to a pendant group as described herein), D is R₇ (an end group, as defined herein); and when Y is a backbone unit which is attached to L or Z, D is a covalent bond or a linking group attaching Y to L or Z. The linking group may optionally be —O—, —S—, arylene, sulfinyl, sulfonyl, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, or amino.

R₄-R₇ are each independently hydrogen, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azide, azo, phosphate phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, or amino.

In some embodiments, the polymeric moiety is a copolymer moiety wherein at least one type of monomeric unit comprises a targeting moiety (according to any of the respective embodiments described herein) and at least one type of monomeric unit does not comprise such a targeting moiety. The distribution of a monomeric unit comprising a targeting moiety may be in accordance with any distribution described herein of a monomeric unit in a copolymer moiety (e.g., random, alternating, periodic copolymer, and/or block copolymer).

In some embodiments of any of the embodiments described herein wherein a portion of monomeric units comprise a targeting moiety, the monomeric units comprising a targeting moiety are, on average, closer to a terminus of the polymeric moiety distal to the lipid moiety, e.g., an average distance (as measured in atoms or backbone units along the backbone of the polymeric moiety) of monomeric units comprising a targeting moiety from the lipid moiety is greater than an average distance of the other monomeric units from the lipid moiety.

In some embodiments, at least a portion (and optionally all) of the monomeric units comprising a targeting moiety form a block (of one or more monomeric units) near (and optionally at) a terminus of the polymeric moiety distal to the lipid moiety. In some such embodiments, the copolymer moiety contains a single monomeric unit which comprises a targeting moiety, and the monomeric unit is at a terminus of the polymeric moiety distal to the lipid moiety.

Without being bound by any particular theory, it is assumed that a targeting moiety located distal to the lipid moiety may be more effective as a targeting moiety (e.g., more effective at binding to a target), for example, due to the targeting moiety being less sterically shielded (e.g., by a surface to which the lipid moiety is associated) and therefore more exposed to and thus better able to make contact with targets in an aqueous environment.

In alternative embodiments, the polymeric moiety does not comprise a targeting moiety described herein according to any of the respective embodiments.

In some embodiments of any of the embodiments described herein, the percentage of backbone units (represented by the variable Y) which are substituted by the pendant group represented by (-L-Z) (as represented by the formula 100%*n/(n+m)) is at least 20%. In some embodiments, the percentage of backbone units substituted by the aforementioned pendant group is at least 30%. In some embodiments, the percentage of backbone units substituted by the aforementioned pendant group is at least 40%. In some embodiments, the percentage of backbone units substituted by the aforementioned pendant group is at least 50%. In some embodiments, the percentage of backbone units substituted by the aforementioned pendant group is at least 60%. In some embodiments, the percentage of backbone units substituted by the aforementioned pendant group is at least 70%. In some embodiments, the percentage of backbone units substituted by the aforementioned pendant group is at least 80%. In some embodiments, the percentage of backbone units substituted by the aforementioned pendant group is at least 90%. In some embodiments, the percentage of backbone units substituted by the aforementioned pendant group is at least 95%. In some embodiments, the percentage of backbone units substituted by the aforementioned pendant group is at least 98%.

In some embodiments of any of the embodiments described herein, m is 0, such that each of the backbone units (represented by the variable Y) is substituted by the pendant group represented by (-L-Z).

In some embodiments of any of the embodiments described herein, n is at least 5. In some embodiments, n is at least 10. In some embodiments, n is at least 15.

In some embodiments of any of the embodiments described herein, n is in a range of from 2 to 1,000. In some embodiments of any of the embodiments described herein, n is in a range of from 2 to 500. In some embodiments of any of the embodiments described herein, n is in a range of from 2 to 200. In some embodiments of any of the embodiments described herein, n is in a range of from 2 to 100. In some embodiments of any of the embodiments described herein, n is in a range of from 2 to 50. In some such embodiments, m is 0.

In some embodiments of any of the embodiments described herein, n is in a range of from 3 to 1,000. In some embodiments of any of the embodiments described herein, n is in a range of from 3 to 500. In some embodiments of any of the embodiments described herein, n is in a range of from 3 to 200. In some embodiments of any of the embodiments described herein, n is in a range of from 3 to 100. In some embodiments of any of the embodiments described herein, n is in a range of from 3 to 50. In some embodiments of any of the embodiments described herein, n is in a range of from 5 to 50. In some embodiments of any of the embodiments described herein, n is in a range of from 10 to 50. In some embodiments of any of the embodiments described herein, n is in a range of from 10 to 25. In some such embodiments, m is 0.

In some embodiments of any of the embodiments described herein, m is in a range of from 0 to 1,000. In some such embodiments, n is in a range of from 2 to 1,000, such that the total number of backbone units is in a range of from 2 to 2,000. In some such embodiments, n is in a range of from 3 to 1,000. In some embodiments, n is in a range of from 3 to 500. In some embodiments, n is in a range of from 3 to 200. In some embodiments, n is in a range of from 3 to 100. In some embodiments, n is in a range of from 3 to 50. In some embodiments, n is in a range of from 5 to 50. In some embodiments, n is in a range of from 10 to 50.

In some embodiments of any of the embodiments described herein, m is in a range of from 0 to 500. In some such embodiments, n is in a range of from 2 to 1,000. In some such embodiments, n is in a range of from 3 to 1,000. In some embodiments, n is in a range of from 3 to 500. In some embodiments, n is in a range of from 3 to 200. In some embodiments, n is in a range of from 3 to 100. In some embodiments, n is in a range of from 3 to 50. In some embodiments, n is in a range of from 5 to 50. In some embodiments, n is in a range of from 10 to 50.

In some embodiments of any of the embodiments described herein, m is in a range of from 0 to 200. In some such embodiments, n is in a range of from 2 to 1,000. In some such embodiments, n is in a range of from 3 to 1,000. In some embodiments, n is in a range of from 3 to 500. In some embodiments, n is in a range of from 3 to 200. In some embodiments, n is in a range of from 3 to 100. In some embodiments, n is in a range of from 3 to 50. In some embodiments, n is in a range of from 5 to 50. In some embodiments, n is in a range of from 10 to 50.

In some embodiments of any of the embodiments described herein, m is in a range of from 0 to 100. In some such embodiments, n is in a range of from 2 to 1,000. In some such embodiments, n is in a range of from 3 to 1,000. In some embodiments, n is in a range of from 3 to 500. In some embodiments, n is in a range of from 3 to 200. In some embodiments, n is in a range of from 3 to 100. In some embodiments, n is in a range of from 3 to 50. In some embodiments, n is in a range of from 5 to 50. In some embodiments, n is in a range of from 10 to 50.

In some embodiments of any of the embodiments described herein, m is in a range of from 0 to 50. In some such embodiments, n is in a range of from 2 to 1,000. In some such embodiments, n is in a range of from 3 to 1,000. In some embodiments, n is in a range of from 3 to 500. In some embodiments, n is in a range of from 3 to 200. In some embodiments, n is in a range of from 3 to 100. In some embodiments, n is in a range of from 3 to 50. In some embodiments, n is in a range of from 5 to 50. In some embodiments, n is in a range of from 10 to 50.

In some embodiments of any of the embodiments described herein, m is in a range of from 0 to 20. In some such embodiments, n is in a range of from 2 to 1,000. In some such embodiments, n is in a range of from 3 to 1,000. In some embodiments, n is in a range of from 3 to 500. In some embodiments, n is in a range of from 3 to 200. In some embodiments, n is in a range of from 3 to 100. In some embodiments, n is in a range of from 3 to 50. In some embodiments, n is in a range of from 5 to 50. In some embodiments, n is in a range of from 10 to 50.

In some embodiments of any of the embodiments described herein, m is in a range of from 0 to 10. In some such embodiments, n is in a range of from 2 to 1,000. In some such embodiments, n is in a range of from 3 to 1,000. In some embodiments, n is in a range of from 3 to 500. In some embodiments, n is in a range of from 3 to 200. In some embodiments, n is in a range of from 3 to 100. In some embodiments, n is in a range of from 3 to 50. In some embodiments, n is in a range of from 5 to 50. In some embodiments, n is in a range of from 10 to 50.

The lipid moiety (represented by the variable X in formula I herein) according to any of the embodiments in this section may be attached to a polymeric moiety according to any of the embodiments described in the section herein relating to the polymeric moiety.

The lipid moiety may optionally be derived from any lipid known in the art (including, but not limited to, a naturally occurring lipid). Derivation of the lipid moiety from the lipid may optionally consist of substituting a hydrogen atom at any position of the lipid with the polymeric moiety represented in general formula I by [Y(-L-Z)]n[Y]m (i.e., the polymeric moiety represented by general formula Ia).

In some embodiments of any of the embodiments described herein, the lipid moiety (according to any of the respective embodiments described herein) is attached to a Y(-L-Z) unit (according to any of the embodiments described herein relating to Y, L and/or Z), that is, backbone unit substituted by the pendant group described herein (e.g., rather than a backbone unit not substituted by the pendant group).

Alternatively or additionally, in some embodiments of any of the embodiments described herein wherein m is at least 1, the lipid moiety (according to any of the respective embodiments described herein) may optionally be attached to a backbone unit (Y) which is not substituted by a pendant group described herein (e.g., rather than attached to a backbone unit substituted by the pendant group). For example, the polymeric moiety may optionally be a copolymer wherein the identity of the backbone unit attached to the lipid moiety varies randomly between molecules.

Thus, the depiction of X in Formula I as being attached to a backbone unit substituted by a pendant group (i.e., Y-(L-Z)) rather than to an unsubstituted backbone unit Y is arbitrary, and is not intended to be limiting.

In some embodiments of any of the embodiments described herein, the lipid moiety is a moiety of a lipid which is a fatty acid, a monoglyceride, a diglyceride, a triglyceride, a glycerophospholipid, a sphingolipid, or a sterol. In some embodiments, the lipid is a glycerophospholipid.

In some embodiments of any of the embodiments described herein, the lipid moiety comprises at least one fatty acid moiety (e.g., an acyl group derived from a fatty acid). The fatty acid moiety may be derived from a saturated or unsaturated fatty acid. For example, the lipid moiety may consist of a fatty acid moiety, or be a monoglyceride moiety comprising one fatty acid moiety, a diglyceride moiety comprising two fatty acid moieties, or a triglyceride moiety comprising three fatty acid moieties.

Examples of fatty acid moieties which may optionally be comprised by the lipid moiety include, without limitation, lauroyl, myristoyl, palmitoyl, stearoyl, palmitoleoyl, oleoyl, and linoleoyl.

Suitable examples of glycerophospholipids include, without limitation, a phosphatidyl ethanolamine, a phosphatidyl serine, a phosphatidyl glycerol and a phosphatidyl inositol.

In some embodiments of any of the embodiments described herein, the lipid moiety represented by the variable X has the general formula III:

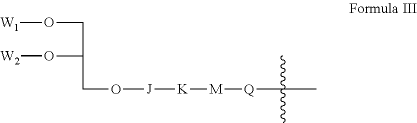

W₁ and W₂ are each independently hydrogen, alkyl, alkenyl, alkynyl or acyl, wherein at least one of W₁ and W₂ is not hydrogen;
J is —P(═O)(OH)—O—, or J is absent (such that K is attached directly to the depicted oxygen atom of a glycerol moiety);
K is a substituted or unsubstituted hydrocarbon from 1 to 10 carbon atoms in length;
M is a linking group which is —O—, —S—, amino, sulfinyl, sulfonyl, phosphate, phosphonyl, phosphinyl, carbonyl, thiocarbonyl, urea, thiourea, carbamyl, thiocarbamyl, amido, carboxy, or sulfonamide, or M is absent (such that K is attached directly to Q); and
Q is a substituted or unsubstituted hydrocarbon from 1 to 10 carbon atoms in length, or Q is absent.
Q is attached to a backbone unit of the polymeric backbone according to any of the respective embodiments described herein, or alternatively, when Q is absent, M is attached to the aforementioned backbone unit.

When M is absent, Q is also absent, and K is attached to a backbone unit of the polymeric backbone according to any of the respective embodiments described herein.

In some embodiments of any of the embodiments described herein, one of W₁ and W₂ is hydrogen and the other is not hydrogen.

In some embodiments of any of the embodiments described herein, neither W₁ nor W₂ is hydrogen.

In some embodiments of any of the embodiments described herein, at least one of W₁ and W₂ is an alkyl, alkenyl, alkynyl or acyl, which is from 10 to 30 carbon atoms in length. In some embodiments, each of W₁ and W₂ is from 10 to 30 carbon atoms in length.

Examples of acyl groups which may optionally serve independently as W₁ and/or W₂ include, without limitation, lauroyl, myristoyl, palmitoyl, stearoyl, palmitoleoyl, oleoyl, and linoleoyl.

In some embodiments of any of the embodiments described herein, J is —P(═O)(OH)—O— (e.g., the lipid moiety is a glycerophospholipid).

Herein, the length of the hydrocarbon represented by the variable K refers to the number of atoms separating J and M (i.e., along the shortest path between J and M) as depicted in formula III.

When K is a substituted hydrocarbon, M may be attached to a carbon atom of the hydrocarbon per se, or be attached to a substituent of the hydrocarbon.

In some embodiments of any of the embodiments described herein, K is an acyl moiety (e.g., —C(═O)—C(CH₃)₂—). In some such embodiments, J is absent, such that K is attached directly to the depicted oxygen atom of a glycerol moiety. In some such embodiments, K comprises a carbonyl linking group (—C(═O)—), which attaches to the oxygen atom of a glycerol moiety via an ester bond.

In some embodiments of any of the embodiments described herein, K is an ethanolamine moiety (e.g., —CH₂—CH₂—NH—, or —CH₂—CH₂— attached to a nitrogen atom), a serine moiety (e.g., —CH₂—CH(CO₂H)—NH—, or —CH₂—CH(CO₂H)— attached to a nitrogen atom), a glycerol moiety (e.g., —CH(OH)—CH(OH)—CH—O—) and an inositol moiety (e.g., -cyclohexyl(OH)₄—O—). In some embodiments, J is —P(═O)(OH)—O—.

In some embodiments of any of the embodiments described herein, M is amido, optionally —C(═O)NH—.

In some embodiments, the nitrogen atom of the amido is attached to K. In some such embodiments, K is an ethanolamine or serine moiety described herein.

In some embodiments of any of the embodiments described herein, Q is a substituted or unsubstituted methylene group. In some such embodiments, M is amido. In some embodiments, the C(═O) of the amido is attached to Q.

In some embodiments of any of the embodiments described herein, Q is a methylene group substituted by one or two substituents. In some embodiments, the methylene group is substituted by one or two alkyl groups (e.g., C₁₋₄-alkyl).

In some embodiments of any of the embodiments described herein, Q is a methylene group substituted by two substituents. In some embodiments, the methylene group is substituted by two alkyl groups (e.g., C₁₋₄-alkyl). In some embodiments, the alkyl groups are methyl, such that Q is dimethylmethylene (—C(CH₃)₂—).

In some embodiments of any of the embodiments described herein, M and Q are each absent, and K is terminated by a substituted or unsubstituted methylene group, according to any of the respective embodiments described herein with respect to Q, for example, a methylene group substituted by two substituents (e.g., dimethylmethylene (—C(CH₃)₂—)). In some embodiments, K further comprises a carbonyl group according to any of the respective embodiments described herein.

In some embodiments of any of the embodiments described herein, J, M and Q are each absent. In some such embodiments, K comprises a carbonyl linking group (—C(═O)—) attached directly to the depicted oxygen atom of a glycerol moiety (via an ester bond), and further comprises a substituted or unsubstituted methylene group (e.g., dimethylmethylene). In some embodiment, K consists of a carbonyl linking group attached directly to the depicted oxygen atom of a glycerol moiety (via an ester bond), and a substituted or unsubstituted methylene group, for example, K is —C(═O)—C(CH₃)₂—.

In some embodiments of any of the embodiments described herein, at least a portion of the monomeric units comprise a targeting moiety (according to any of the embodiments described herein relating to a targeting moiety).

Herein, a “targeting moiety” refers to a moiety which is capable of bringing a compound (e.g., a compound according to some embodiments of the invention) into proximity with a selected substance and/or material (which is referred to herein as a “target”). The target is optionally a cell (e.g., a proliferating cell associated with the proliferative disease or disorder), wherein the proximity is such that the targeting moiety facilitates attachment and/or internalization of the compound into a target cell, and such that the compound may exert a therapeutic effect.

In any of the embodiments described herein wherein m is at least 1, at least a portion of the monomeric units comprising a targeting moiety (the number of which is represented by the variable m), according to any of the respective embodiments described herein, are monomeric units which do not comprise the pendant group represented by (-L-Z). In some such embodiments, each of the monomeric units comprising a targeting moiety (according to any of the respective embodiments described herein) is a monomeric unit which comprises the pendant group represented by (-L-Z) (i.e., a backbone unit Y substituted by (-L-Z)), that is, none of the monomeric units comprising the pendant group represented by (-L-Z) comprise the aforementioned targeting moiety.

In any of the embodiments described herein wherein m is at least 1, each of the monomeric units which do not comprise the pendant group represented by (-L-Z) (the number of which is represented by the variable m) comprises a targeting moiety (according to any of the respective embodiments described herein). In some such embodiments, each of the monomeric units comprising a targeting moiety (according to any of the respective embodiments described herein) is a monomeric unit which does not comprise the pendant group represented by (-L-Z), that is, none of the monomeric units comprising the pendant group represented by (-L-Z) comprise the aforementioned targeting moiety, and each of the monomeric units which does not comprise the pendant group represented by (-L-Z) comprises the aforementioned targeting moiety.

In any of the embodiments described herein wherein m is at least 1, a monomeric unit comprising a targeting moiety may consist essentially of a backbone unit Y (according to any of the respective embodiments described herein) substituted by one or more targeting moieties (according to any of the respective embodiments described herein).

The backbone unit Y of a monomeric unit comprising a targeting moiety may optionally be different (optionally considerably different) in structure than a backbone unit Y of other monomeric units in the polymeric moiety (according to any of the respective embodiments described herein).

In any of the embodiments described herein wherein m is at least 1, the polymeric moiety comprises a monomeric unit which comprises a targeting moiety, and the monomeric unit is at a terminus of the polymeric moiety distal to the lipid moiety. In such embodiments, the compound represented by general formula I has the formula Ib:

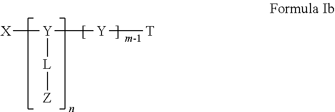

wherein:
T is a monomeric unit comprising a targeting moiety (according to any of the respective embodiments described herein);
X and T are attached to distal termini of the moiety represented by [Y(-L-Z)]n[Y]m−1; and
X, Y, L, Z, n and m are defined in accordance with any of the embodiments described herein relating to general formula I, with the proviso that m is at least 1.

It is to be understood that T in formula Ib is a type of monomeric unit represented by Y (i.e., without the pendant group represented by (-L-Z)) in formulas I and Ia, and the number of monomeric units represented by Y (i.e., without the pendant group represented by (-L-Z)) other than T is represented by the value m−1, such that the total number of monomeric units without the pendant group represented by (-L-Z)), including T, is represented by the variable m, as in formulas I and Ia.

In some embodiments, m is 1, such that m−1 is zero, and the compound represented by formula Ib consequently has the formula: X—[Y(-L-Z)]n—T, wherein L, T, X, Y, Z and n are defined in accordance with any of the embodiments described herein.

A monomeric unit comprising a targeting moiety according to any of the respective embodiments described herein may optionally be prepared by preparing a monomer comprising a targeting moiety, and using the monomer to prepare a polymeric moiety described herein (e.g., by polymerization of monomers according to any of the respective embodiments described herein) and/or by modifying a monomeric unit in a polymeric moiety subsequently to preparation of a polymeric moiety (e.g., by polymerization of monomers according to any of the respective embodiments described herein), using any suitable technique known in the art, including, but not limited to, techniques for conjugation.

In some embodiments of any of the embodiments described herein relating to a targeting moiety, the targeting moiety does not comprise a moiety having general formula II (according to any of the respective embodiments described herein). For example, even if a moiety represented by general formula II is capable of forming a bond with a target as described herein, the phrase “targeting moiety”, in some embodiments, is to be understood as relating to a moiety distinct from a moiety represented by variable Z (having general formula II).

In some embodiments of any one of the embodiments described herein, the pendant group represented by (-L-Z) is selected so as not to form a bond with the target and/or so as not to include a structure and/or property of a targeting moiety as described herein in any one of the respective embodiments. For example, in embodiments wherein a targeting moiety comprising a nucleophilic group (according to any of the respective embodiments described herein)—for example, an amine group—is capable of forming a bond (e.g., covalent bond) with a target, the variable Z (having general formula II) is optionally selected such that the depicted amine/ammonium group is a tertiary amine/ammonium (i.e., no more than one of R₁-R₃ is hydrogen) or quaternary ammonium (i.e., none of R₁-R₃ is hydrogen), preferably a quaternary ammonium (e.g., comprising a trimethylamino group, such as in phosphocholine). Tertiary amine groups, and especially quaternary ammonium groups, may be significantly less reactive nucleophilic groups than primary and secondary amine groups.

In some embodiments of any of the embodiments described herein relating to a targeting moiety, the targeting moiety comprises (and optionally consists of) at least one functional group capable of forming a covalent bond or non-covalent bond (preferably a selective non-covalent bond) with a substance and/or material (which is referred to herein as a “target”), e.g., at a surface of the target (e.g., a surface of a cell and/or tissue).

Herein, the phrase “functional group” encompasses chemical groups and moieties of any size and any functionality described herein (for example, any functionality capable of forming a covalent bond or non-covalent bond with a target).

A non-covalent bond according to any of the respective embodiments described herein may optionally be effected by non-covalent interactions such as, without limitation, electrostatic attraction, hydrophobic bonds, hydrogen bonds, and aromatic interactions.

In some embodiments, the targeting moiety comprises a functional group capable of forming a non-covalent bond which is selective for the target, e.g., an affinity (e.g., as determined based on a dissociation constant) of the targeting moiety and/or functional group to the target is greater than an affinity of the of the targeting moiety and/or functional group to most (or all) other compounds capable of forming a non-covalent bond with the targeting moiety.

In some embodiments of any one of the embodiments described herein, the functional group(s) are capable of forming a covalent bond with one or more specific functional groups (e.g., hydroxy, amine, thiohydroxy and/or oxo groups) which are present on the target (e.g., a target according to any of the respective embodiments described herein).

Examples of functional groups (in a targeting moiety) capable of forming a covalent bond with a target (according to any of the respective embodiments described herein) and the type of covalent bonds they are capable of forming, include, without limitation:
nucleophilic groups such as thiohydroxy, amine (e.g., primary or secondary amine) and hydroxy, which may form covalent bonds with, e.g., a nucleophilic leaving group (e.g., any nucleophilic group described herein), Michael acceptor (e.g., any Michael acceptor described herein), acyl halide, isocyanate and/or isothiocyanate (e.g., as described herein) in a target;
nucleophilic leaving groups such as halo, azide (—N₃), sulfate, phosphate, sulfonyl (e.g. mesyl, tosyl), N-hydroxysuccinimide (NHS) (e.g. NHS esters), sulfo-N-hydroxysuccinimide, and anhydride, which may form covalent bonds with, e.g., a nucleophilic group (e.g., as described herein) in a target;

Michael acceptors such as enones (e.g., maleimide, acrylate, methacrylate, acrylamide, methacrylamide), nitro groups and vinyl sulfone, which may form covalent bonds with, e.g., a nucleophilic group (e.g., as described herein) in a target, optionally thiohydroxy; dihydroxyphenyl groups (according to any of the respective embodiments described herein), which may form covalent bonds with, e.g., a nucleophilic group (e.g., as described herein) and/or a substituted or unsubstituted phenyl group (e.g., another dihydroxyphenyl group) in a target, as described herein;
an acyl halide (—C(═O)-halogen), isocyanate (—NCO) and isothiocyanate (—N═C═S) group, which may form covalent bonds with, e.g., a nucleophilic group (e.g., as described herein) in a target;
a carboxylate (—C(═O)OH) group, which may form a covalent bond with, e.g., a hydroxyl group in a target to form an ester bond and/or an amine group (e.g., primary amine) in a target to form an amide bond (optionally by reaction with a coupling reagent such as a carbodiimide); and/or a carboxylate group is in a target and may form an amide or ester bond with an amine or hydroxyl group, respectively, in the targeting moiety;
an oxo group (optionally in an aldehyde group (—C(═O)H)), which may form a covalent imine bond with an amine group (e.g., a primary amine) in a target; and/or an oxo group (optionally in an aldehyde group) is in a target and may form a covalent imine bond with an amine groups in the targeting moiety; and/or
thiohydroxy groups, which may form covalent disulfide (—S—S—) bonds with a thiohydroxy group in a target.

Modification of a monomer (e.g., prior to polymerization) or a monomeric unit of a polymeric moiety (e.g., subsequent to polymerization) to comprise any of the functional groups described herein may optionally be performed using any suitable technique for conjugation known in the art. The skilled person will be readily capable of selecting a suitable technique for any given molecule to be modified.

Herein, the term “dihydroxyphenyl” refers to an aryl group (as defined herein) which is a phenyl substituted by two hydroxyl groups at any positions thereof. The phenyl may optionally be substituted by additional substituents (which may optionally comprise additional hydroxyl groups), to thereby form a substituted dihydroxyphenyl group; or alternatively, the phenyl comprises no substituents other than the two hydroxyl groups, such that the dihydroxyphenyl group is an unsubstituted dihydroxyphenyl group.

In some embodiments of any one of the embodiments described herein, the dihydroxyphenyl group is an ortho-dihydroxyphenyl (wherein the hydroxyl groups are attached to the phenyl at adjacent positions) or a para-dihydroxyphenyl (wherein the hydroxyl groups are attached to opposite sides of the phenyl ring), each being a substituted or unsubstituted dihydroxyphenyl. In some such embodiments, the ortho-dihydroxyphenyl or para-dihydroxyphenyl is an unsubstituted dihydroxyphenyl.

A dihydroxyphenyl group according to any of the respective embodiments described herein may optionally bond covalently and/or non-covalently to a target according to any one or more attachment mechanism described for dihydroxyphenyl (catechol) groups in Lee et al. [PNAS 2006, 103:12999-13003], Brodie et al. [Biomedical Materials 2011, 6:015014] and/or International Patent Application PCT/IL2015/050606, the contents of each of which are incorporated in their entirety, and especially contents regarding bonds formed by dihydroxyphenyl (catechol) groups to surfaces.

In some embodiments of any one of the embodiments described herein, the functional group capable of forming a bond to a target is a functional group capable of forming a covalent bond with an amine group, optionally a primary amine group. In some such embodiments, the target comprises on or more amino acids or amino acid residues, for example, a peptide or polypeptide of any length (e.g., at least two amino acid residues, for example, proteins), and the amine groups may optionally be lysine side chain amine groups and/or N-terminal amine groups. In some embodiments, the target comprises an extracellular matrix protein, for example, collagen. In some embodiments, the target comprises cartilage (e.g., articular cartilage).

In some embodiments of any one of the embodiments described herein, the targeting moiety comprises (and optionally consists of) at least one functional group capable of forming a non-covalent bond with the target (e.g., as described herein in any one of the respective embodiments).

In some embodiments of any one of the embodiments described herein, a functional group capable of forming a non-covalent bond with the target comprises (and optionally consists of) a polysaccharide and/or polypeptide (e.g., a protein and/or fragment thereof), wherein the target optionally comprises a ligand of the polysaccharide and/or polypeptide; and/or the target comprises a polysaccharide and/or polypeptide (e.g., a protein and/or fragment thereof) and the functional group capable of forming a non-covalent bond with the target is a ligand of the polysaccharide and/or polypeptide.

Examples of suitable polysaccharides and/or polypeptides, and ligands thereof, include, without limitation: avidin or streptavidin as a polypeptide described herein, and biotin as a ligand thereof; a polysaccharide-binding polypeptide as a polypeptide described therein, and a complementary polysaccharide as a ligand thereof (or a complementary polysaccharide-binding polypeptide as a ligand of a polysaccharide described herein); a collagen-binding polypeptide as a polypeptide described therein, and a complementary collagen as a ligand thereof (or a collagen as a polypeptide described herein and a complementary collagen-binding polypeptide as a ligand thereof); a cell receptor expressed by a cell, and a ligand selectively bound by the receptor; an antibody towards any antigen (e.g., wherein the target described herein optionally comprises the antigen) or a fragment of such an antibody as a polypeptide described herein, and the respective antigen as a ligand thereof; and an antibody mimetic towards any antigen (e.g., wherein the target described herein optionally comprises the antigen).

Examples of cell receptors expressed by a cell include, without limitation, receptors characteristic of a particular type of cell and/or tissue, and receptors overexpressed by a cancer cell. The cell receptor or the cell is optionally a target described herein, and the targeting moiety optionally comprises any ligand of the receptor. Examples of such ligands include, without limitation, transferrin, a ligand of transferrin receptor which may optionally target transferrin receptor overexpressed by some cancer cells; keratinocyte growth factor (KGF or FGF7) which is specific for cells of epithelial origin, and may optionally target KGF receptor such as that overexpressed by an endometrial carcinoma or pancreatic carcinoma [Visco et al., Int J Oncol 1999, 15:431-435; Siegfried et al., Cancer 1997, 79:1166-1171]; and epidermal growth factor (EGF) which may optionally target an EGF receptor, optionally an erbB, such as that overexpressed by gliomas and endometrial carcinomas [Normanno et al., Curr Drug Targets 2005, 6:243-257]).

As used herein, the term “antibody” encompasses any type of immunoglobin.

As used herein, the phrase “antibody mimetic” encompasses any type of molecule, optionally a polypeptide, referred as such in the art capable of selectively binding an antigen (e.g., non-covalently). Non-limiting examples of antibody mimetics include affibodies, affilins, affimers, affitins, alphabodies, anticalins, avimers, DARPins, Fynomers, Kunitz domain peptides, and monobodies, e.g., as described in Nygren [FEBS J 2008, 275:2668-2676], Ebersbach et al. [J Mol Biol 2007, 372:172-185], Johnson et al. [Anal Chem 2012, 84:6553-6560], Krehenbrink et al. [J Mol Biol 2008, 383:1058-1068], Desmet et al. [Nature Comm 2014, 5:5237], Skerra [FEBS J 2008, 275:2677-2683], Silverman et al. [Nature Biotechnol 2005, 23:1556-1561], Stumpp et al. [Drug Discov Today 2008, 13:695-701], Grabulovski et al. [J Biol Chem 2007, 282:3196-3204], Nixon & Wood [Curr Opin Drug Discov Devel 2006, 9:261-268], Koide & Koide [Methods Mol Biol 2007, 325:95-109], and Gebauer & Skerra [Curr Opin Chem Biol 2009, 13:245-255], the contents of each of which are incorporated in their entirety, and especially contents regarding particular types of antibody mimetics.

As used herein, the phrase “polysaccharide-binding polypeptide” encompasses any polypeptide or oligopeptide (peptide chains of at least 2, and preferably at least 4 amino acid residues in length) capable of selectively binding (e.g., non-covalently) to a polysaccharide. A wide variety of polysaccharide-binding polypeptides and their binding specificities will be known to the skilled person, and include short peptide sequences (e.g., from 4 to 50, optionally 4 to 20 amino acid residues in length), and longer polypeptides such as proteins or fragments (e.g., carbohydrate-binding modules and/or domains) thereof. In addition, the phrase “polysaccharide-binding polypeptide” encompasses antibodies capable of specifically binding to a polysaccharide. Such antibodies will be available to the skilled person and/or the skilled person will know how to prepare such antibodies, using immunological techniques known in the art.

Examples of polysaccharide-binding polypeptides which may be used in some of any one of the embodiments of the invention include, without limitation, carbohydrate-binding modules (CBMs); and hyaluronic acid-binding peptides, polypeptides and/or modules (e.g., having a sequence as described in any of International Patent Application publication WO 2013/110056; International Patent Application publication WO 2014/071132; Barta et al. [Biochem J 1993, 292:947-949], Kohda et al. [Cell 1996, 86:767-775], Brisset & Perkins [FEBS Lett 1996, 388:211-216], Peach et al. [J Cell Biol 1993, 122:257-264], Singh et al. [Nature Materials 2014, 13:988-995], and Zaleski et al. [Antimicrob Agents Chemother 2006, 50:3856-3860], the contents of each of which are incorporated in their entirety, and especially contents regarding particular polysaccharide-binding polypeptides, for example, a hyaluronic acid-binding peptide sequence.

Examples of CBMs which may be used in some of any one of the embodiments of the invention, include, without limitation, CBMs belonging to the families CBM3, CBM4, CBM9, CBM10, CBM17 and/or CBM28 (which may optionally be used to bind cellulose, e.g., in a cellulose-containing target); CBM5, CBM12, CBM14, CBM18, CBM19 and/or CBM33 (which may optionally be used to bind chitin and/or other polysaccharides comprising N-acetylglucosamine, e.g., in a chitin-containing target); CBM15 (which may optionally be used to bind hemicellulose, e.g., in a hemicellulose-containing target); and/or CBM20, CBM21 and/or CBM48 (which may optionally be used to bind starch and/or glycogen, e.g., in a starch-containing and/or glycogen-containing target).

As used herein, the phrase “collagen-binding polypeptide” encompasses any polypeptide or oligopeptide (peptide chains of at least 2, and preferably at least 4 amino acid residues in length) capable of selectively binding (e.g., non-covalently) to a collagen (e.g., one type of collagen, some types of collagen, all types of collagen), including glycosylated polypeptides and oligopeptides such as peptidoglycans and proteoglycans. A wide variety of collagen-binding polypeptides and their binding specificities will be known to the skilled person, and include short peptide sequences (e.g., from 4 to 50, optionally 4 to 20 amino acid residues in length), and longer polypeptides such as proteins or fragments (e.g., collagen-binding domains) thereof. In addition, the phrase “collagen-binding polypeptide” encompasses antibodies capable of specifically binding to a collagen. Such antibodies will be available to the skilled person and/or the skilled person will know how to prepare such antibodies, using immunological techniques known in the art.

Examples of collagen-binding polypeptides which may be used in embodiments of the invention include, without limitation, collagen-binding proteins (e.g., decorin), fragments thereof and/or other polypeptides as described in U.S. Pat. No. 8,440,618, Abd-Elgaliel & Tung [Biopolymers 2013, 100:167-173], Paderi et al. [Tissue Eng Part A 2009, 15:2991-2999], Rothenfluh et al. [Nat Mater 2008, 7:248-254] and Helms et al. [J Am Chem Soc 2009, 131:11683-11685] (the contents of each of which are incorporated in their entirety, and especially contents regarding particular collagen-binding polypeptides.

It is expected that during the life of a patent maturing from this application many relevant functional groups and moieties for binding will be developed and/or uncovered and the scope of the terms “targeting moiety”, “functional group”, “cell receptor”, “antibody”, “antibody mimetic”, “collagen-binding polypeptide” and “polysaccharide-binding polypeptide” and the like is intended to include all such new technologies a priori.

In some embodiments of any of the embodiments described herein, a functional group in a targeting moiety (according to any of the respective embodiments described herein) is attached to a linking group (as defined herein). The linking group may optionally be any linking group or linking moiety described herein, including, without limitation, a substituted or unsubstituted hydrocarbon. In some embodiments, the targeting moiety (optionally a substituent of a backbone unit Y) consists essentially of a functional group attached to the rest of the polymeric moiety via the linking group.

A functional group may optionally be attached to the linking moiety by a covalent bond obtainable by a reaction between two functional groups, for example, any covalent bond and/or functional groups described herein in the context of forming a covalent bond between a functional group and a target.

A targeting moiety in a liposome according to any of the respective embodiments described herein may optionally be a targeting moiety according to any of the respective embodiments described herein. A targeting moiety in a liposome may be comprised by a polymeric compound according to some embodiments of the invention (according to any of the respective embodiments described herein), the liposome comprising the polymeric compound. Alternatively or additionally, a targeting moiety in a liposome may optionally be comprised by another compound in the liposome, optionally a bilayer-forming lipid (according to any of the respective embodiments described herein) conjugated to a targeting moiety according to any of the respective embodiments described herein.

Herein, a “targeting agent” refers to a compound (“agent”) comprising (and optionally consisting essentially of) a targeting moiety according to any of the respective embodiments described herein (e.g., in the context of a targeting moiety comprised by a polymeric compound described herein). Typically, the phrase “targeting agent” is used to refer to a compound other than a polymeric compound comprising a targeting moiety, as described herein.

In some embodiments, a functional moiety (e.g., targeting moiety or labeling moiety) is covalently attached to a liposome. Such attachment may be obtained in some embodiments by using techniques known in the art (e.g., amide bond formation).

### Therapeutically Active Agent:

According to the present embodiments, the liposome as described in any of the embodiments or in any combination thereof further comprises at least one therapeutically active agent.

Herein, the phrase “therapeutically active agent” refers to any agent (e.g., compound, compounds) having a therapeutic effect, as well as to any portion of an agent (e.g., a moiety of a compound) which generates an agent having a therapeutic effect upon release (e.g., upon cleavage of one or more covalent bonds).

According to some of any of the embodiments described herein, the therapeutically active agent is bound to a surface of the liposome and/or within a lipid bilayer and/or core of the liposome, as defined herein in any of the embodiments.

Herein, the phrase “bound to a surface” refers to attachment or adherence to the outer surface or outer layer of the liposome, as defined herein in any of the embodiments.

Herein, the phrase “within a lipid bilayer” refers to a state of being embedded within the lipid bilayer of the liposome as defined herein in any of the embodiments, optionally as an integral part of the lipid bilayer of the liposome.

Herein, the phrase “within a core” refers to a state of being located inside the void formed within the bilayer of the liposome, as defined herein in any of the embodiments.

According to the present embodiments, the therapeutically active agent is associated with the liposome, by being incorporated in a liposome and/or on a surface of the liposome. The therapeutically active agent may, for example, be attached by a covalent or non-covalent (e.g., electrostatic and/or hydrophobic) bond to a liposome (e.g., to an exterior surface and/or interior surface of a liposome membrane), incorporated within a liposome membrane (e.g., a lipophilic agent which stably partitions to a lipid phase of the liposome), and/or enveloped within a core of a liposome (e.g., a hydrophilic agent in an aqueous compartment of the liposome).

Herein, the phrase “incorporated in a liposome” refers to a therapeutically active agent as described in any of the embodiments herein, which is either incorporated within a lipid bilayer and/or within a core of a liposome, as described in any of the embodiments.

Herein, the phrase “on a surface of the liposome” refers to a therapeutically active agent as described in any of the embodiments herein, which is bound to a surface of the liposome, as described in any of the embodiments.

According to some of any of the embodiments described herein, the therapeutically active agent is effective in treating biofilm, as described and defined herein, and/or in treating a medical condition associated with the biofilm, as described and defined herein.

According to some of any of the embodiments described herein, the liposome, as described herein in any of the embodiments, comprises a therapeutically active agent which is usable in the treatment of biofilm, as described and defined herein and/or is effective in treating biofilm as defined and described herein, and/or is effective in treating a medical condition associated with the biofilm as defined and described herein.

According to some of any of the embodiments described herein, the therapeutically active agent is an antimicrobial agent, preferably, an antimicrobial agent that is capable of treating a biofilm to be treated, as described and defined herein, either alone or when combined with an additional therapeutically active agent. For example, for a biofilm formed of a certain bacterial strain, a therapeutically active agent, or a combination of two or more agents as described herein, that is effective in treating (e.g., eradicating, inhibiting growth, reducing a load) of the bacterial strain or a biofilm formed thereof can be selected.

The phrase “antimicrobial” as used herein, refers to a property of a substance (e.g., a compound or a composition) that can effect a parameter of microorganism, as defined herein, including death, eradication, elimination, reduction in number, reduction of growth rate, inhibition of growth, change in population distribution of one or more species of microbial life forms. This term encompasses antibacterial agents, which are also referred to herein as antibiotics, as well as, for example, anti-mycobacterial agent, antiviral agents, anti-fungal agents, anti-protozoal agents, and anti-parasitic agents.

Non-limiting examples of conventional antifungal agents include polyene-based antifungal agents such as amphotericin, amphotericin B, nystatin and pimaricin, azole-based antifungal agents such as fluconazole, itraconazole and ketoconazole, allylamine- or morpholine-based antifungal agents such as allylamines (naftifine, terbinafine), and antimetabolite-based antifungal agents such as 5-fluorocytosine, and fungal cell wall inhibitor such as echinocandins like caspofungin, micafungin and anidulafungin.

Exemplary antibacterial agents, or antibiotics, include, without limitations, aminoglycosides (e.g., gentamicin, tobramycin, amikacin, streptomycin), fluoroquinolones (e.g., ciprofloxacin, levofloxacin, gatifloxacin, moxifloxacin), carbapenems (e.g., imipenem, meropenem, ertapenem), polymyxins (e.g., colistin, polymyxin B, polymyxin E), tetracyclinea (e.g., tetracycline, doxycycline), macrolides (e.g., azithromycin, clarithromycin, erythromycin), beta-lactams (e.g., ampicillin, amoxicillin, ticarcillin, piperacillin, imipenem, oxacillin, cephalosporins), sulfonamides (e.g., sulfamethoxazole, sulfadiazine, sulfisoxazole, sulfacetamide, sulfamethazine, sulfasalazine), rifamycins (e.g., rifampin, rifabutin), nitroimidazoles (e.g., metronidazole, tinidazole), phosphonic acid antibiotics (e.g., fosfomycin), chloramphenicol, glycopeptides (e.g., vancomycin), oxazolidinones (e.g., linezolid), cephalosporins (e.g., cefazolin, ceftriaxone, cephalothin, ceftazidime, cefepime), monobactams (e.g., aztreonam), nitrofurans (e.g., nitrofurantoin), polyphenols (e.g., ellagic acid and derivative thereof) [see, e.g., M. Daglia, Current Opinion in Biotechnology, 23(2), 2012, 174-181] and lipopeptides (e.g., daptomycin).

Non-limiting examples of conventional antibacterial agents (antibiotics) include, but are not limited to, gentamicin, ampicillin, amikacin (AK), cefazolin, ceftriaxone, clindamycin, cephalothin, ciprofloxacin, chloramphenicol, ceftazidime (CAZ), cefepime (CPE), erythromycin, trimethoprim/sulfamethoxazole (T/S), gatifloxacin, piperacillin/tazobactam (P/T), aztreonam (AZT), imipenem, levofloxacin, penicillin, oxacillin, nitrofurantoin, linezolid, moxifloxacin, meropenem (MER), tobramycin (TO), ciprofloxacin (CP), tetracycline, vancomycin, rifampin, synercid, streptomycin, colistin (CT) and chloramphenicol (C).In some of any of the embodiments described herein, the therapeutically active agent is an anti-inflammatory agent.

Non-limiting examples of anti-inflammatory agents include, but are not limited to, steroids (e.g., corticosteroids such as cortisone, prednisone), non-steroidal anti-inflammatory drugs (NSAIDs, e.g., ibuprofen, aspirin, naproxen), COX-2 inhibitors (e.g., celecoxib), biologics (e.g., TNF inhibitors), and antihistamines.

In some of any of the embodiments described herein, the therapeutically active agent is or comprises an anti-biofilm agent, which are also referred to herein and in the art as anti-biofouling agents.

Exemplary compounds useable as anti-biofilm agents include, but are not limited to, antibiotics as described herein, dispersants (i.e., compounds that can penetrate or break down the extracellular matrix of biofilms, optionally making the biofilm more susceptible to antimicrobial agents) (e.g., dispersin B, D-amino acids, DNase, proteases), quorum sensing inhibitors (QSIs as known in the art; e.g., N-acyl homoserine lactone inhibitors), EDTA and N-acetylcysteine.

In some of any of the embodiments described herein, a combination of two or more therapeutically active agents are included in and/or on the surface of the same liposome as described herein. In some of any of the embodiments described herein, a combination of two or more therapeutically active agents are used by co-administering two or more liposomes as described herein in any of the respective embodiments.

In some of any of the embodiments described herein, a combination of two or more therapeutically active agents as described herein, provides an additive effect.

As used herein, the term “additive effect” refers to a combined effect of two or more agents which is equal to the sum of the individual effects of each agent.

In some of any of the embodiments described herein, a combination of two or more therapeutically active agents as described herein, provides a synergistic effect.

As used herein, the term “synergistic effect” refers to a combined effect of two or more agents which is greater than the sum of the individual effects of each agent. Synergy can be determined by methods well-known in the art, for example, using isobolograms.

In exemplary embodiments, the one or more therapeutically active agent(s) comprise a sulfonamide antibiotic, for example, sulfamethoxazole (SMX).

In exemplary embodiments, the one or more therapeutically active agent(s) comprise an antimicrobial polyphenol, for example, ellagic acid (EA).

In exemplary embodiments, one of the therapeutically active agents is a sulfonamide antibiotic, for example, sulfamethoxazole (SMX), and another one of the therapeutically active agents is an antimicrobial polyphenol, for example, ellagic acid (EA), which acts in synergy with the sulfonamide antibiotic, for example, sulfamethoxazole (SMX).

A liposome that comprises a therapeutically active agent as described herein is also referred to herein as a drug-loaded liposome.

### Drug Delivery:

According to an aspect of some embodiments of the invention, there is provided a liposome comprising: at least one bilayer-forming lipid; a polymeric compound; optionally a positively-charged lipidic agent; optionally a sterol; and a therapeutically active agent, as described herein in any of the respective embodiments and any combination thereof, which is for use in delivering the therapeutically active agent to a subject in need thereof. The liposome can be used per se or be formulated in a pharmaceutical composition as described herein.

According to an aspect of some embodiments of the present invention, there is provided a method of delivering a therapeutically active agent to a subject in need thereof, the method comprising administering to the subject a liposome comprising at least one bilayer-forming lipid; a polymeric compound; optionally a positively-charged lipidic agent; optionally a sterol; and a therapeutically active agent, as described herein in any of the respective embodiments and any combination thereof, or a composition comprising the liposome, as described herein, thereby delivering the therapeutically active agent.

According to an aspect of some embodiments of the present invention, there is provided a use of a liposome comprising at least one bilayer-forming lipid; a polymeric compound; optionally a positively-charged lipidic agent; optionally a sterol; and a therapeutically active agent, as described herein in any of the respective embodiments and any combination thereof, in the manufacture of a medicament for delivering the therapeutically active agent to a subject in need thereof.

As used herein, “delivery” or “delivering” of a therapeutically active agent or drug (which terms are used herein interchangeably) refers to administration of the therapeutically active agent to a subject while controlling duration and/or proportion of the agent at a desired bodily site, depending on a subject's condition (e.g., a bodily site at which the agent desirably exerts a therapeutic effect). Thus, the terms “delivery” and “delivering” (and grammatical variations thereof) encompass targeting of a therapeutically active agent to a specific bodily site, such that a higher proportion of the agent reaches the bodily site (e.g., using a suitable targeting moiety); and/or control over duration of a presence of such an agent in the body (e.g., in the blood)—for example, by sustained release—which may be associated with a duration of such an agent at a desired bodily site (even if in the absence of specific targeting to the bodily site).

As used herein, the phrase “bodily site” includes any organ, tissue, membrane, cavity, blood vessel, tract, biological surface or muscle, which contacting therewith (e.g., delivering thereto or applying thereon) the liposome or the therapeutically active agent disclosed herein is beneficial. Exemplary bodily sites include, but are not limited to, the skin, a dermal layer, the scalp, an eye, an ear, a mouth, a throat, a stomach, a small intestines tissue, a large intestines tissue, a kidney, a pancreas, a liver, the digestive system, the respiratory tract, a bone marrow tissue, a mucosal membrane, a nasal membrane, the blood system, a blood vessel, a muscle, a pulmonary cavity, an artery, a vein, a capillary, a heart, a heart cavity, a male or female reproductive organ and any visceral organ or cavity. Any organ or tissue onto which microorganism can exist in contemplated.

As used herein, “sustained release” refers to a formulation of an agent which provides a gradual and/or delayed (“sustained”) release of the agent (e.g., from a reservoir such a liposome according to any of the respective embodiments described herein), which results in the agent being present in a bodily site (e.g., in the blood upon systemic administration, or in a bodily site to which the agent is locally administered) for a longer duration and/or at a later time (relative to administration) than if the agent is administered per se (via the same administration route).

In some embodiments, the sustained release is characterized by a concentration of therapeutically active agent (e.g., in the blood upon systemic administration, or in a bodily site to which the agent is locally administered) which is at least half of the maximal concentration (Cmax) for a time period which is at least 50% more than a corresponding time period (i.e., during which a concentration of an agent is at least half of the maximal concentration) an agent upon administration of the therapeutically effective agent per se (e.g., as defined herein) in an amount which results in the same maximal concentration. In some such embodiments, the time period (for sustained release) is at least 100% more than (i.e., twice) a corresponding time period (for the agent per se). In some embodiments, the time period (for sustained release) is at least 200% more than (i.e., 3-fold) a corresponding time period (for the agent per se). In some embodiments, the time period (for sustained release) is at least 400% more than (i.e., 5-fold) a corresponding time period (for the agent per se).

Sustained release (according to any of the respective embodiments described herein), may allow, for example, for a regimen characterized by less frequent administration and/or by greater therapeutic efficacy of any given administration. The skilled person will be readily capable of determining a suitable frequency of administration for a given therapeutically active agent based on the duration of the sustained release (e.g., a time period during which the concentration of the agent is at least half of the maximal concentration, according to any of the respective embodiments described herein, and/or at least a minimal effective concentration), and the ratio between a desirable maximal concentration and a minimal effective concentration for the given agent (e.g., a “therapeutic window” of the agent).

### Pharmaceutical Compositions:

The liposome of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the liposomes comprising a therapeutically active agent accountable for the biological effect.

Hereinafter, the phrases “physiologically acceptable carrier” and “pharmaceutically acceptable carrier”, which may be interchangeably used, refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences”, Mack Publishing Co., Easton, PA, latest edition, which is incorporated herein by reference.

Suitable routes of administration may, for example, include oral, rectal, topical, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

In some of any of the embodiments described herein, the tissue encompasses any part of a living organism, a bodily site or a living organ of a subject.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For topical administration, an appropriate carrier may be selected and optionally other ingredients that can be included in the composition, as is detailed herein. Hence, the compositions can be, for example, in a form of a cream, an ointment, a paste, a gel, a lotion, and/or a soap.

Ointments are semisolid preparations, typically based on vegetable oil (e.g., shea butter and/or cocoa butter), petrolatum or petroleum derivatives. As with other carriers or vehicles, an ointment base should be inert, stable, nonirritating and non-sensitizing.

Lotions are preparations that may to be applied to the skin without friction. Lotions are typically liquid or semiliquid preparations with a water or alcohol base, for example, an emulsion of the oil-in-water type. Lotions are typically preferred for treating large areas (e.g., as is frequently desirable for sunscreen compositions), due to the ease of applying a more fluid composition.

Creams are viscous liquids or semisolid emulsions, either oil-in-water or water-in-oil. Cream bases typically contain an oil phase, an emulsifier and an aqueous phase. The oil phase, also called the “lipophilic” phase, optionally comprises petrolatum and/or a fatty alcohol such as cetyl or stearyl alcohol. The aqueous phase optionally contains a humectant. The emulsifier in a cream formulation is optionally a nonionic, anionic, cationic or amphoteric surfactant.

Herein, the term “emulsion” refers to a composition comprising liquids in two or more distinct phases (e.g., a hydrophilic phase and a lipophilic phase). Non-liquid substances (e.g., dispersed solids and/or gas bubbles) may optionally also be present.

As used herein and in the art, a “water-in-oil emulsion” is an emulsion characterized by an aqueous phase which is dispersed within a lipophilic phase.

As used herein and in the art, an “oil-in-water emulsion” is an emulsion characterized by a lipophilic phase which is dispersed within an aqueous phase.

Pastes are semisolid dosage forms which, depending on the nature of the base, may be a fatty paste or a paste made from a single-phase aqueous gel. The base in a fatty paste is generally petrolatum, hydrophilic petrolatum, and the like. The pastes made from single-phase aqueous gels generally incorporate carboxymethylcellulose or the like as a base.

Gel formulations are semisolid, suspension-type systems. Single-phase gels optionally contain organic macromolecules distributed substantially uniformly throughout the carrier liquid, which is typically aqueous; but also, preferably, contains a non-aqueous solvent, and optionally an oil. Preferred organic macromolecules (e.g., gelling agents) include cross-linked acrylic acid polymers such as the family of carbomer polymers, e.g., carboxypolyalkylenes, that may be obtained commercially under the trademark Carbopol®. Other types of preferred polymers in this context are hydrophilic polymers such as polyethylene oxides, polyoxyethylene-polyoxypropylene copolymers and polyvinyl alcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

A composition formulated for topical administration may optionally be present in a patch, a swab, a pledget, and/or a pad.

Dermal patches and the like may comprise some or all of the following components: a composition to be applied (e.g., as described herein); a liner for protecting the patch during storage, which is optionally removed prior to use; an adhesive for adhering different components together and/or adhering the patch to the skin; a backing which protects the patch from the outer environment; and/or a membrane which controls release of a drug to the skin.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinylpyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (drug-loaded liposomes) effective to prevent, alleviate or ameliorate symptoms of a disorder (e.g., bacterial infection) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See, for example, Fingl, et al., 1975, in “The Pharmacological Basis of Therapeutics”, Ch. 1, p. 1).

Dosage amount and interval may be adjusted individually to provide the infected tissue with levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein, the term “subject” includes mammals, preferably human beings at any age which suffer from the pathology. Preferably, this term encompasses individuals who are at risk to develop the pathology.

In some of any of the embodiments described herein or in any combination thereof, the pharmaceutical composition comprises a plurality of liposomes as described in any of the embodiments herein.

In some of any of the embodiments described herein, at least a portion of the liposomes each liposome is a liposome as described in any of the embodiments herein.

In some of any of the embodiments described herein, in at least one portion of the liposomes each liposome is a liposome as described in any of the embodiments herein, comprising a first therapeutically active agent bound to a surface of the liposome and/or within a lipid bilayer and/or core of the liposome as described herein, and in at least one another portion of the liposomes each liposome is a liposome as described in any of the embodiments herein, comprising a second therapeutically active agent bound to a surface of the liposome and/or within a lipid bilayer and/or core of the liposome as described herein. In some of any of the embodiments, the first and second therapeutically active agents being different from one another. In some embodiments, the pharmaceutical composition comprises three or more types of drug-loaded liposomes, and at least one type of the drug-loaded liposomes are as described herein in any of the respective embodiments and any combination. In some embodiments, all of the three or more types of drug-loaded liposomes are as described herein, and each comprises a different therapeutically active agent.

According to an aspect of some embodiments of the invention, there is provided a pharmaceutical composition as described herein in any of the respective embodiments and any combination thereof, for use in treating biofilm in a subject in need thereof, as described and defined herein.

According to an aspect of some embodiments of the invention, there is provided a method of treating biofilm in a subject in need thereof, as described and defined herein, the method comprising administering to the subject a pharmaceutical composition as described herein in any of the respective embodiments and any combination thereof.

According to some of any of these embodiments, treating the biofilm is effected by administering two or more pharmaceutical compositions, each comprising a different type of drug-loaded liposomes, at least one type are the liposomes as described herein in any of the respective embodiments and any combination thereof, which comprise a first therapeutically active agent. The other pharmaceutical compositions can include liposomes as described herein, which comprises a different, second, therapeutically active agent, which can be such that acts in synergy with the first therapeutically active agent. Alternatively, the other pharmaceutical compositions can include liposomes other than described herein, which can comprises the same or different, therapeutically active agent. The other pharmaceutical compositions can comprise one or more other therapeutically active agent(s), in a free form or in a form other than a liposome as described herein, and can comprise such agents that act in synergy with the first therapeutically active agent.

In exemplary such embodiments, the one or more therapeutically active agent(s) comprise a sulfonamide antibiotic, for example, sulfamethoxazole (SMX).

In exemplary such embodiments, the one or more therapeutically active agent(s) comprise an antimicrobial polyphenol, for example, ellagic acid (EA).

In exemplary such embodiments, one of the therapeutically active agents is a sulfonamide antibiotic, for example, sulfamethoxazole (SMX), and another one of the therapeutically active agents is an antimicrobial polyphenol, for example, ellagic acid (EA), which acts in synergy with the sulfonamide antibiotic, for example, sulfamethoxazole (SMX).

In some of any of the embodiments described herein, the two or more pharmaceutical compositions as described herein can be administered to a subject in need thereof, concomitantly, sequentially or alternately.

### Biofilm Treatment:

According to an aspect of some embodiments of the invention, there is provided a liposome, comprising a bilayer-forming lipid; a polymeric compound; optionally a positively-charged lipidic agent; optionally a sterol; and a therapeutically active agent, as described herein in any of the respective embodiments or any combination thereof, or a pharmaceutical composition comprising same, as described herein, for use in treating biofilm in a subject in need thereof.

According to an aspect of some embodiments of the present invention, there is provided a method of treating biofilm in a subject in need thereof, the method comprising administering to the subject a liposome, comprising a bilayer-forming lipid; a polymeric compound; optionally a positively-charged lipidic agent; optionally a sterol; and a therapeutically active agent, as described herein in any of the respective embodiments or any combination thereof, or one or more pharmaceutical compositions as described herein, thereby treating the biofilm in the subject.

According to some of any of the embodiments described herein, the method and uses as described herein are for treating a medical conditions associated with biofilm formation, as described herein.

The term “biofilm”, as used herein and in the art, refers to an aggregate of living cells which are stuck to each other and/or immobilized onto a surface as colonies. The cells are frequently embedded within a self-secreted matrix of extracellular polymeric substance (EPS), also referred to as “slime”, which is a polymeric sticky mixture of nucleic acids, proteins and polysaccharides.

In the context of the present embodiments, the living cells forming a biofilm can be cells of a unicellular microorganism (prokaryotes, archaea, bacteria, eukaryotes, protists, fungi, algae, euglena, protozoan, dinoflagellates, apicomplexa, trypanosomes, amoebae and the likes), or cells of multicellular organisms in which case the biofilm can be regarded as a colony of cells (like in the case of the unicellular organisms) or as a lower form of a tissue.

In the context of the present embodiments, the cells are of microorganism origins, and the biofilm is a biofilm of microorganisms, such as bacteria and/or fungi. The cells of a microorganism growing in a biofilm are physiologically distinct from cells in the “planktonic form” of the same organism, which by contrast, are single-cells that may float or swim in a liquid medium. Biofilms can go through several life-cycle steps which include initial attachment, irreversible attachment, one or more maturation stages, and dispersion.

The phrase “anti-biofilm treatment” or “anti-biofouling treatment” and any grammatical variation thereof refers to the capacity of a substance (e.g., a liposome as described herein or a composition comprising same) to effect the load of a biofilm of bacterial, fungal and/or other cells, at an infected bodily site, by reducing a load, inhibiting the formation and/or reducing in the rate of buildup, of a biofilm of bacterial, fungal and/or other cells), and/or by disrupting the biofilm or by eradicating the biofilm.

In some of any of the embodiments described herein or in any combination thereof, treating the biofilm is affected by reducing a load of a biofilm, and/or interfering with a formation of biofilm, and/or disrupting a formation of a biofilm, and/or eradicating a biofilm, in a subject in need thereof, for example, at a bodily site of the subject in which biofilm was formed or which is at risk of biofilm formation (e.g., is infected by a biofilm-forming pathogenic microorganism).

Herein, the phrase “reducing a load” refers to a decrease in the number of the microorganism(s) (e.g., of a biofilm), or to a decrease in the rate of their growth or both in the substrate as compared to a non-treated substrate.

In some embodiments of any of the embodiments described herein, biofilm load is defined as an area of the biofilm.

In some embodiments of any of the embodiments described herein, biofilm load is defined as a mass and/or volume of the biofilm.

In some embodiments of any of the embodiments described herein, biofilm load is defined as a number of cells in the biofilm.

The biofilm load may optionally be determined using any technique known in the art for detecting and quantifying an amount of cells and/or microorganisms in a biofilm. Exemplary such techniques are described herein and include single cells fluorescence microscopy for visualizing liposomal fusion with bacteria cells, determination of bacterial viability in biofilms by, e.g., LIVE/DEAD™ staining, and paraffin-embedded thin sectioning followed by staining (e.g., using LIVE/DEAD™ for, e.g., fluorescence imaging.

In some of any of the embodiments described herein or in any combination thereof, treating the biofilm is effected by treating a subject in need thereof with at least one pharmaceutical composition as described herein in any of the respective embodiments.

In some of any of the embodiments described herein, reducing, preventing disrupting and/or eradicating the load of a biofilm in a subject is effected topically by applying a composition containing the active ingredient(s) on a wound. This method is also effective in reducing, preventing and/or disrupting a biofilm formation on the wound dressing used to treat the wound.

In some of any of the embodiments described herein, the biofilm treatment is effected in the presence of, or is mediated by, divalent ions, for example, divalent cations such as calcium ions, which are concentrated in the vicinity of the biofilm.

Treatment can be effected by administering the liposomes as described herein in any of the respective embodiments and any combination to an infected bodily site, to thereby treat the biofilm or the medical condition associated therewith.

By “infected bodily site” it is meant a tissue or an organ having a biofilm formed therein or thereof.

The liposomes can be co-administered with an additional agent that is capable of treating biofilm and/or a medical condition as defined herein. Co-administration can be simultaneous, sequential or alternating (i.e., administered at different time intervals).

Medical conditions associated with biofilm include, but are not limited to, chronic wounds, ear infections, urinary tract infections, dental plaque, and certain types of lung infections such as cystic fibrosis, and bronchiectasis. Additional such conditions may also include implant-related infections and device-related infections such as catheter-associated urinary tract infections and prosthetic joint infections.

In some of any of the embodiments described herein, the biofilm is a bacterial biofilm. In some embodiments, the biofilm is formed of bacterial cells (or from a bacterium).

In some embodiments, the phrase “pathogenic microorganism” refers to a disease-causing bacterium (or a bacterial strain). In some embodiments, a biofilm is formed of bacterial cells of bacteria selected from the group consisting of all Gram-positive and Gram-negative bacteria.

The terms “bacterium” or “bacteria”, as used herein, refer to all prokaryotic organisms, including those within all of the phyla in the Kingdom Procaryotae. It is intended that these terms encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within these terms are prokaryotic organisms that are Gram-negative or Gram-positive. “Gram-negative” and “Gram-positive” refer to staining patterns with the Gram-staining process, which is well known in the art. (See e.g., Finegold and Martin, Diagnostic Microbiology, 6th Ed., CV Mosby St. Louis, pp. 13-15 (1982)). “Gram-positive bacteria” are bacteria that retain the primary dye used in the Gram stain, causing the stained cells to generally appear dark blue to purple under the microscope. “Gram-negative bacteria” do not retain the primary dye used in the Gram stain, but are stained by the counterstain. Thus, Gram-negative bacteria generally appear red. In some embodiments, bacteria are continuously cultured. In some embodiments, bacteria are uncultured and existing in their natural environment (e.g., at the site of a wound or infection) or obtained from patient tissues (e.g., via a biopsy). Bacteria may exhibit pathological growth or proliferation.

Non-limiting examples of bacteria include bacteria of a genus selected from the group including Salmonella, Shigella, Escherichia, Enterobacter, Serratia, Proteus, Yersinia, Citrobacter, Edwardsiella, Providencia, Klebsiella, Hafnia, Ewingella, Kluyvera, Morganella, Planococcus, Stomatococcus, Micrococcus, Staphylococcus, Vibrio, Aeromonas, Plesiomonas, Haemophilus, Actinobacillus, Pasteurella, Mycoplasma, Ureaplasma, Rickettsia, Coxiella, Rochalimaea, Ehrlichia, Streptococcus, Enterococcus, Aerococcus, Gemella, Lactococcus, Leuconostoc, Pedicoccus, Bacillus, Corynebacterium, Arcanobacterium, Actinomyces, Rhodococcus, Listeria, Erysipelothrix, Gardnerella, Neisseria, Campylobacter, Arcobacter, Wolinella, Helicobacter, Achromobacter, Acinetobacter, Agrobacterium, Alcaligenes, Chryseomonas, Comamonas, Eikenella, Flavimonas, Flavobacterium, Moraxella, Oligella, Pseudomonas, Shewanella, Weeksella, Xanthomonas, Bordetella, Franciesella, Brucella, Legionella, Afipia, Bartonella, Calymmatobacterium, Cardiobacterium, Streptobacillus, Spirillum, Peptostreptococcus, Peptococcus, Sarcinia, Coprococcus, Ruminococcus, Propionibacterium, Mobiluncus, Bifidobacterium, Eubacterium, Lactobacillus, Rothia, Clostridium, Bacteroides, Porphyromonas, Prevotella, Fusobacterium, Bilophila, Leptotrichia, Wolinella, Acidaminococcus, Megasphaera, Veilonella, Norcardia, Actinomadura, Norcardiopsis, Streptomyces, Micropolyspora, Thermoactinomyces, Mycobacterium, Treponema, Borrelia, Leptospira and Chlamydia.

In some of any of the embodiments of the present invention the pathogenic bacteria are of one or more of the following species: Acinetobacter baumannii, Helicobacter pylori, Burkholderia multivorans, Canipylobacter jejuni, Deinococcus radiodurans, E. coli, Enterobacter cloacae, Enterococcus faecalis, Haemophilus influenzae, Klebsiella pneumoniae, Klebsiella oxytoca, Mycobacterium tuberculosis, Neisseria gonorrhoeae, Neisseria meningitidis, Pseudomonas aeruginosa, Pseudomonas phosphoreui, Escherichia coli, Bacillus Subtifis, Borrelia burgfrferi, Yersinia pestis, Deinococcus radiodurans, Mycobacterium tuberculosis, Enterococcus faecalis, Streptococcus pneumoniae, Streptococcus pyogenes and Staphylococcus aureus, Salmonella typhimuriunim, Serratia marcescens, Staphylococcus aureus, Staphylococcus epidermidis, Staphylococcus pneumoniae, Staphylococcus sanguis, Staphylococcus viridans, Vibrio harveyi, Vibrio cholerae, Vibrio parahaeniolyticus, Vibrio alginolyticus, Yersinia enterocolitica or Yersinia pestis, including any strain or mutant thereof.

In some of any of the embodiments, the bacterial biofilm is formed by a pathogenic bacterium such as, but are not limited to, Pseudomonas, Escherichia, Klebsiella, Enterobacter, Acinetobacter, Serratia, Haemophilus, Chlamydia, Salmonella, Arsenophonus, Cosenzaea, Moraxella, Brucella, Bordetella, Vibrio, Campylobacter, Legionella, Francisella, Photorhabdus, Neisseria, Proteus, Shigella, Edwardsiella, Plesiomonas, Aeromonas, Alcaligenes, Providencia, Yersinia, Staphylococcus, Bacillus, Listeria, Streptococcus, Gardnerella, Cronobacter, Enterococcus, Clostridium, Corynebacterium, Mycobacterium, Micrococcus, Hafnia, Morganella, Pasteurella, Mycoplasma, Ureaplasma, Coxiella, Borrelia, Aerococcus, Lactococcus, Actinomyces, Rhodococcus, Propionibacterium, Bartonella, Lactobacillus, Bifidobacterium, Rothia, Porphyromonas, Prevotella, Bacteroides, Fusobacterium, Megasphaera, Acidaminococcus, Deinococcus, Helicobacter, Burkholderia, Canipylobacter, Veilonella, Norcardia, Treponema, Leptospira, Micropolyspora and Thermoactinomyces.

In some embodiments, the Gram-negative biofilm-forming bacteria may be selected from the group of pathogenic Gram-negative biofilm-forming bacteria such as, but not limited to, at least one of Pseudomonas, Escherichia, Klebsiella, Enterobacter, Acinetobacter, Serratia, Haemophilus, Chlamydia, Salmonella, Arsenophonus, Veilonella, Cosenzaea, Moraxella, Brucella, Bordetella, Vibrio, Campylobacter, Legionella, Francisella, Prevotella, Acidaminococcus, Megasphaera, Fusobacterium, Photorhabdus, Neisseria, Proteus, Shigella, Bacteroides, Porphyromonas, Edwardsiella, Plesiomonas, Aeromonas, Alcaligenes, Providencia, Pasteurella, Hafnia, Morganella, Mycoplasma, Ureaplasma, Coxiella, Leptospira, Treponema, Borrelia, Aerococcus, Lactococcus, Bartonella, Yersinia, Deinococcus, Helicobacter, Burkholderia, Canipylobacter, Micropolyspora and Thermoactinomyces.

In some embodiments, the Gram-positive biofilm-forming bacteria may be selected from the group of pathogenic Gram-positive biofilm-forming bacteria such as, but not limited to, at least one of Staphylococcus, Bacillus, Listeria, Streptococcus, Gardnerella, Cronobacter, Enterococcus, Rothia, Lactobacillus, Clostridium, Corynebacterium, Mycobacterium, Norcardia, Rhodococcus, Propionibacterium, Bifidobacterium, Actinomyces, and Micrococcus.

In some embodiment, the microorganism is a fungus, selected from the group of pathogenic fungi such as, but not limited to, Candida, Aspergillus, Fusarium, Cryptococcus, Rhizopus, Trichophyton, Malassezia and Pneumocystis.

In exemplary embodiments, a biofilm is formed of Pseudomonas aeruginosa bacterial cells.

In some embodiments, inhibiting, reducing and/or retarding a load of a biofilm as described herein is reflected by reducing biofilm load in a subject in need thereof by at least 20%, at least 30%, at least 40%, at least 50%, at least 60%, at least 70%, at least 80%, at least 90%, including any value therebetween, compared to the same subject in need thereof prior to treatment with the drug-loaded liposomes as described herein in any of the respective embodiments.

In some embodiments, inhibiting, reducing and/or retarding the load of a biofilm in or on a substrate or an article containing the substrate, is effected by administering to a subject in need thereof, as defined herein in any of the embodiments, an anti-fouling effective amount of a liposome of a composition comprising same, as described herein.

As used herein, “an anti-fouling effective amount” is defined as the amount which is sufficient to inhibit, retard and/or reduce the load of a biofilm as described herein in a subject in need thereof as described herein. Assays for determining an anti-fouling effective amount are known is the art and are contemplated herein.

In some of any of the embodiments, a biofilm is formed of pathogenic fungi.

Representative examples of pathogenic fungi, against which a compound having general Formula A as described herein can be efficiently used according to the present embodiments include, without limitation, fungi of the genus Absidia: Absidia corymbifera; genus Ajellomyces: Ajellomyces capsulatus, Ajellomyces dermatitidis; genus Arthroderma: Arthroderma benhamiae, Arthroderma fulvum, Arthroderma gypseum, Arthroderma incurvatum, Arthroderma otae, Arthroderma vanbreuseghemii; genus Aspergillus: Aspergillus flavus, Aspergillus fumigatus, Aspergillus niger; genus Blastomyces: Blastomyces dermatitidis; genus Candida: Candida albicans, Candida glabrata, Candida guilliermondii, Candida krusei, Candida parapsilosis, Candida tropicalis, Candida pelliculosa; genus Cladophialophora: Cladophialophora carrionii; genus Coccidioides: Coccidioides immitis; genus Cryptococcus: Cryptococcus neoformans; genus Cunninghamella: Cunninghamella sp.; genus Epidermophyton: Epidermophyton floccosum; genus Exophiala: Exophiala dermatitidis; genus Filobasidiella: Filobasidiella neoformans; genus Fonsecaea: Fonsecaea pedrosoi; genus Fusarium: Fusarium solani; genus Geotrichum: Geotrichum candidum; genus Histoplasma: Histoplasma capsulatum; genus Hortaea: Hortaea werneckii; genus Issatschenkia: Issatschenkia orientalis; genus Madurella: Madurella grisae; genus Malassezia: Malassezia furfur, Malassezia globosa, Malassezia obtusa, Malassezia pachydermatis, Malassezia restricta, Malassezia slooffiae, Malassezia sympodialis; genus Microsporum: Microsporum canis, Microsporum fulvum, Microsporum gypseum; genus Mucor: Mucor circinelloides; genus Nectria: Nectria haematococca; genus Paecilomyces: Paecilomyces variotii; genus Paracoccidioides: Paracoccidioides brasiliensis; genus Penicillium: Penicillium marneffei; genus Pichia, Pichia anomala, Pichia guilliermondii; genus Pneumocystis: Pneumocystis carinii; genus Pseudallescheria: Pseudallescheria boydii; genus Rhizopus: Rhizopus oryzae; genus Rhodotorula: Rhodotorula rubra; genus Scedosporium: Scedosporium apiospermum; genus Schizophyllum: Schizophyllum commune; genus Sporothrix: Sporothrix schenckii; genus Trichophyton: Trichophyton mentagrophytes, Trichophyton rubrum, Trichophyton verrucosum, Trichophyton violaceum; and of the genus Trichosporon: Trichosporon asahii, Trichosporon cutaneum, Trichosporon inkin, Trichosporon mucoides.

In some of any of the embodiments, a biofilm is formed of pathogenic parasites and protozoa.

Representative examples of pathogenic parasites and protozoa include, but are not limited to, various types of amoeba, Leishmania spp, Plasmodium falciparum: Trypanosoma cruzi (causing Chagas' disease), Trypanosoma bucei (causing “sleeping sickness”), Plasmodium vivax (causing malaria), Cryptosporidium parvum (causing cryptosporidiosis), Cyclospora cayetanensis, Giardia lamblia (causing giardiasis) and many others.

In some of any of the embodiments described herein, the biofilm is formed in an air-exposed tissue or organ, for example, in a lung tissue, a skin tissue or a mucosal tissue (e.g., skin); and/or in a systemic tissue which is not exposed to air.

### Additional Definitions:

Herein, the term “hydrocarbon” describes an organic moiety that includes, as its basic skeleton, a chain of carbon atoms, substituted mainly by hydrogen atoms. The hydrocarbon can be saturated or non-saturated, be comprised of aliphatic, alicyclic or aromatic moieties, and can optionally be substituted by one or more substituents (other than hydrogen). A substituted hydrocarbon may have one or more substituents, whereby each substituent group can independently be, for example, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, oxo, cyano, nitro, azo, azide, sulfonamide, carboxy, thiocarbamate, urea, thiourea, carbamate, amide, and hydrazine. The hydrocarbon can be an end group or a linking group, as these terms are defined herein. The hydrocarbon moiety is optionally interrupted by one or more heteroatoms, including, without limitation, one or more oxygen, nitrogen and/or sulfur atoms. In some embodiments of any of the embodiments described herein relating to a hydrocarbon, the hydrocarbon is not interrupted by any heteroatoms.

Preferably, the hydrocarbon moiety has 1 to 20 carbon atoms. Whenever a numerical range; e.g., “1 to 20”, is stated herein, it implies that the group, in this case the alkyl group, may contain 1 carbon atom, 2 carbon atoms, 3 carbon atoms, etc., up to and including 20 carbon atoms.

Herein, the term “alkyl” describes a saturated aliphatic hydrocarbon end group, as defined herein, including straight chain and branched chain groups. Preferably, the alkyl group has 1 to 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted. Substituted alkyl may have one or more substituents, whereby each substituent group can independently be, for example, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine.

The term “alkylene” describes a saturated aliphatic hydrocarbon linking group, as this term is defined herein, which differs from an alkyl group, as defined herein, only in that alkylene is a linking group rather than an end group.

Herein, the term “alkenyl” describes an unsaturated aliphatic hydrocarbon end group which comprises at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted. Substituted alkenyl may have one or more substituents, whereby each substituent group can independently be, for example, cycloalkyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine.

Herein, the term “alkynyl” describes an unsaturated aliphatic hydrocarbon end group which comprises at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted. Substituted alkynyl may have one or more substituents, whereby each substituent group can independently be, for example, cycloalkyl, alkenyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or non-substituted. Substituted cycloalkyl may have one or more substituents, whereby each substituent group can independently be, for example, alkyl, alkenyl, alkynyl, cycloalkyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine. The cycloalkyl group can be an end group, as this phrase is defined herein, wherein it is attached to a single adjacent atom, or a linking group, as this phrase is defined herein, connecting two or more moieties.

The term “aryl” describes an all-carbon monocyclic or fused-ring polycyclic (i.e., rings which share adjacent pairs of carbon atoms) end group (as this term is defined herein) having a completely conjugated pi-electron system. The aryl group may be substituted or non-substituted.

Substituted aryl may have one or more substituents, whereby each substituent group can independently be, for example, alkyl, alkenyl, alkynyl, cycloalkyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine. Phenyl and naphthyl are representative aryl end groups.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. Substituted heteroaryl may have one or more substituents, whereby each substituent group can independently be, for example, alkyl, alkenyl, alkynyl, cycloalkyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine. The heteroaryl group can be an end group, as this phrase is defined herein, where it is attached to a single adjacent atom, or a linking group, as this phrase is defined herein, connecting two or more moieties. Representative examples are pyridine, pyrrole, oxazole, indole, purine and the like.

The term “arylene” describes a monocyclic or fused-ring polycyclic linking group, as this term is defined herein, and encompasses linking groups which differ from an aryl or heteroaryl group, as these groups are defined herein, only in that arylene is a linking group rather than an end group.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. Substituted heteroalicyclic may have one or more substituents, whereby each substituent group can independently be, for example, alkyl, alkenyl, alkynyl, cycloalkyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine. The heteroalicyclic group can be an end group, as this phrase is defined herein, where it is attached to a single adjacent atom, or a linking group, as this phrase is defined herein, connecting two or more moieties. Representative examples are piperidine, piperazine, tetrahydrofuran, tetrahydropyran, morpholine and the like.

As used herein, the terms “amine” and “amino” describe both a —NRxRy end group and a —NRx— linking group, wherein Rx and Ry are each independently hydrogen, alkyl, cycloalkyl, aryl, heteroaryl or heteroalicyclic, as these terms are defined herein. When Rx or Ry is heteroaryl or heteroalicyclic, the amine nitrogen atom is bound to a carbon atom of the heteroaryl or heteroalicyclic ring. A carbon atom attached to the nitrogen atom of an amine is not substituted by ═O or ═S, and in some embodiments, is not substituted by any heteroatom.

The amine group can therefore be a primary amine, where both Rx and Ry are hydrogen, a secondary amine, where Rx is hydrogen and Ry is alkyl, cycloalkyl, aryl, heteroaryl or heteroalicyclic, or a tertiary amine, where each of Rx and Ry is independently alkyl, cycloalkyl, aryl, heteroaryl or heteroalicyclic.

The terms “hydroxy” and “hydroxyl” describe a —OH group.

The term “alkoxy” describes both an —O-alkyl and an —O-cycloalkyl end group, or —O— alkylene or —O-cycloalkyl linking group, as defined herein.

The term “aryloxy” describes both an —O-aryl and an —O-heteroaryl end group, or an —O-arylene-linking group, as defined herein.

The term “thiohydroxy” describes a —SH group.

The term “thioalkoxy” describes both an —S-alkyl and an —S-cycloalkyl end group, or —S-alkylene or —S-cycloalkyl linking group, as defined herein.

The term “thioaryloxy” describes both an —S-aryl and an —S-heteroaryl end group, or an —S-arylene-linking group, as defined herein.

The terms “cyano” and “nitrile” describe a —C≡N group.

The term “nitro” describes an —NO₂ group.

The term “oxo” describes a ═O group.

The term “azide” describes an —N═N+=N— group.

The term “azo” describes an —N═N—Rx end group or —N═N=linking group, with Rx as defined herein.

The terms “halide” and “halo” refer to fluorine, chlorine, bromine or iodine.

The term “phosphate” refers to a —O—P(═O)(ORx)—OR_{Y} end group, or to a —O—P(═O)(ORx)—O— linking group, where Rx and R_{Y} are as defined herein.

The terms “phosphonyl” and “phosphonate” refer to an —P(═O)(ORx)—OR_{y} end group, or to a —P(═O)(ORx)—O— linking group, where Rx and R_{Y} are as defined herein. The term “phosphinyl” refers to a —PRxR_{Y} group, where Rx and R_{Y} are as defined hereinabove.

The term “sulfoxide” or “sulfinyl” describes a —S(═O)—Rx end group or —S(═O)— linking group, where Rx is as defined herein.

The terms “sulfonate” and “sulfonyl” describe a —S(═O)₂—Rx end group or —S(═O)₂— linking group, where Rx is as defined herein.

The terms “sulfonamide” and “sulfonamido”, as used herein, encompass both S-sulfonamide and N-sulfonamide end groups, and a —S(═O)₂—NRx— linking group.

The term “S-sulfonamide” describes a —S(═O)₂—NRxR_{Y} end group, with Rx and R_{y} as defined herein.

The term “N-sulfonamide” describes an RxS(═O)₂—NR_{Y}— end group, where Rx and R_{y} are as defined herein.

The term “carbonyl” as used herein, describes a —C(═O)—Rx end group or —C(═O)— linking group, with Rx as defined herein.

The term “acyl” as used herein, describes a —C(═O)—Rx end group, with Rx as defined herein.

The term “thiocarbonyl” as used herein, describes a —C(═S)—Rx end group or —C(═S)— linking group, with Rx as defined herein.

The terms “carboxy” and “carboxyl”, as used herein, encompasses both C-carboxy and O-carboxy end groups, and a —C(═O)—O— linking group.

The term “C-carboxy” describes a —C(═O)—ORx end group, where Rx is as defined herein.

The term “O-carboxy” describes a —OC(═O)—Rx end group, where Rx is as defined herein.

The term “urea” describes a —NRxC(═O)—NRyRw end group or —NRxC(═O)—NRy— linking group, where Rx and Ry are as defined herein and Rw is as defined herein for Rx and Ry.

The term “thiourea” describes a —NRx—C(═S)—NRyRw end group or a —NRx—C(═S)—NRy— linking group, with Rx, Ry and Ry as defined herein.

The terms “amide” and “amido”, as used herein, encompasses both C-amide and N-amide end groups, and a —C(═O)—NRx— linking group.

The term “C-amide” describes a —C(═O)—NRxRy end group, where Rx and Ry are as defined herein.

The term “N-amide” describes a RxC(═O)—NRy— end group, where Rx and Ry are as defined herein.

The term “carbamyl” or “carbamate”, as used herein, encompasses N-carbamate and O-carbamate end groups, and a —OC(═O)—NRx— linking group.

The term “N-carbamate” describes a RyOC(═O)—NRx— end group, with Rx and Ry as defined herein.

The term “O-carbamate” describes an —OC(═O)—NRxRy end group, with Rx and Ry as defined herein.

The term “thiocarbamyl” or “thiocarbamate”, as used herein, encompasses O— thiocarbamate, S-thiocarbamate and N-thiocarbamate end groups, and a —OC(═S)—NRx— or —SC(═O)—NRx— linking group.

The terms “O-thiocarbamate” and “O-thiocarbamyl” describe a —OC(═S)—NRxRy end group, with Rx and Ry as defined herein.

The terms “S-thiocarbamate” and “S-thiocarbamyl” describe a —SC(═O)—NRxRy end group, with Rx and Ry as defined herein.

The terms “N-thiocarbamate” and “N-thiocarbamyl” describe a RyOC(═S)NRx— or RySC(═O)NRx- end group, with Rx and Ry as defined herein.

The term “guanidine” describes a —RxNC(═N)—NRyRw end group or —RxNC(═N)—NRy-linking group, where Rx, Ry and Rw are as defined herein.

The term “hydrazine”, as used herein, describes a —NRx—NRyRw end group or —NRx—NRy— linking group, with Rx, Ry, and Rw as defined herein.

For any of the embodiments described herein, the compound described herein may be in a form of a salt, for example, a pharmaceutically acceptable salt, and/or in a form of a prodrug.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt and/or a base addition salt.

An acid addition salt comprises at least one basic (e.g., amine and/or guanidinyl) group of the compound which is in a positively charged form (e.g., wherein the basic group is protonated), in combination with at least one counter-ion, derived from the selected acid, that forms a pharmaceutically acceptable salt. The acid addition salts of the compounds described herein may therefore be complexes formed between one or more basic groups of the compound and one or more equivalents of an acid.

A base addition salt comprises at least one acidic (e.g., carboxylic acid) group of the compound which is in a negatively charged form (e.g., wherein the acidic group is deprotonated), in combination with at least one counter-ion, derived from the selected base, that forms a pharmaceutically acceptable salt. The base addition salts of the compounds described herein may therefore be complexes formed between one or more acidic groups of the compound and one or more equivalents of a base.

Depending on the stoichiometric proportions between the charged group(s) in the compound and the counter-ion in the salt, the acid additions salts and/or base addition salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof, and/or a carboxylate anion and a base addition salt thereof.

The base addition salts may include a cation counter-ion such as sodium, potassium, ammonium, calcium, magnesium and the like, that forms a pharmaceutically acceptable salt.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a mesylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

As used herein, the term “prodrug” refers to a compound which is converted in the body to an active compound (e.g., the compound of the formula described hereinabove). A prodrug is typically designed to facilitate administration, e.g., by enhancing absorption. A prodrug may comprise, for example, the active compound modified with ester groups, for example, wherein any one or more of the hydroxyl groups of a compound is modified by an acyl group, optionally (C₁₋₄)-acyl (e.g., acetyl) group to form an ester group, and/or any one or more of the carboxylic acid groups of the compound is modified by an alkoxy or aryloxy group, optionally (C₁₋₄)-alkoxy (e.g., methyl, ethyl) group to form an ester group.

Further, each of the compounds described herein, including the salts thereof, can be in a form of a solvate or a hydrate thereof.

The term “solvate” refers to a complex of variable stoichiometry (e.g., di-, tri-, tetra-, penta-hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The compounds described herein can be used as polymorphs and the present embodiments further encompass any isomorph of the compounds and any combination thereof.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

### Materials:

HSPC (Hydrogenated soybean phosphatidylcholine, Mw 786.11), Cholesterol (C₂₇H₄₆O, Mw 386.65), Stearylamine (CH₃(CH₂)₁₇NH₂, Mw 269.509), 18:0 PEG5000 PE (1,2-distearoyl-sn-glycero-3-phosphoethanolamine-N-[methoxy(polyethylene glycol)-5000] (ammonium salt), Mw 5801.071), E. coli extract total (100500P), Lipopolysaccharides from Escherichia coli 0111:B4 (L2630), Magnesium acetate ((CH₃COO)₂Mg, Mw 142.39), Sodium sulfate (Na₂SO₄, Mw 142.04), Calcein (C₃₀H₂₆N₂O₁₃, Mw 622.53), Sulfamethoxazole (C₁₀H₁₁N₃O₃S, Mw 253.28) and Resazurin (C₁₂H₆NNaO₄, Mw 251.17), L-lysine hydrochloride (H₂NCH₂(CD₂)₂CH₂CH(NH₂)CO₂H·HCl, Mw 186.67), Sephadex® G-25 in PD-10 Desalting Columns (GE Life Science), and Float-A-Lyzer® dialysis bags were obtained from Sigma-Aldrich™ (Israel).

Ellagic acid (C₁₄H₆O₈, Mw 302.197) was obtained from Carbosynth Limited (Compton, UK).

DiI Stain (1,10-dioctadecyl-3,3,30,30-etramethylindocarbocyanine perchlorate C₅₉H₉₇ClN₂O₄, Mw 933.88), HPTS dye (8-hydroxypyrene-1,3,6-trisulfonic acid, trisodium salt, C₁₆H₇Na₃O₁₀S₃, Mw 524.37) and LIVE/DEAD™ BacLight™ Bacterial Viability Kit (C₂₇H₃₄I₂N₄, Mw 668.4) were obtained from Thermo Fisher Scientific™ (Waltham, MA, USA).

HPLC-grade water were obtained from J.T. Baker@.

HPLC-grade acetonitrile, 5-Methyl-1(5H)-phenazinone (Pyocyanin), trifluoroacetic acid (TFA) and 1,5-Naphthalenediamine were obtained from Merck@.

HCl was obtained from BioLab™.

All chemicals had high purity and were used without further purification.

HEK293 cells were obtained from The American Type Culture Collection (ATCC, USA).

Pseudomonas aeruginosa strains UCBPP-PA14 and LESB58 were obtained from Bar Ilan University, Israel.

Cell Proliferation Kit (XTT based) was obtained from Biological Industries (Israel Beit Haemek LTD, Israel).

MBEC™ assay system was obtained from MBEC™ Biofilm Technologies Ltd. Calgary, AB, Canada.

Liposome preparation (LUVs), size and zeta-potential characterization: HSPC/cholesterol (40% mol/mol)/pMPC (5 kDa, 5% mol/mol) or HSPC/cholesterol (40% mol/mol)/PEG (5 kDa, 5% mol/mol) or non-functionalized liposomes composed of HSPC/cholesterol (40% mol/mol) were dissolved in chloroform or chloroform/methanol mixture (2:1) and organic solvent was evaporated using first nitrogen stream, followed by 8 hours of vacuum pumping. For introducing a positive charge into liposomes, 5% (mol/mol) of stearylamine was added to chloroform before evaporation. The lipid film was then hydrated with an aqueous solution of (CH₃COO)₂Mg (140 mM, osmolarity=320 mOsm/kg) at 70° C. to reach the desired concentration and solution was gently vortexed. The resulting multilamellar vesicles (MLVs) suspensions were sonicated for 15 minutes at 70° C. to disperse larger aggregates. The vesicles were subsequently downsized by extrusion (Lipex®, Northern Lipids Inc.) through 400 nm, 200 nm, and 100 nm polycarbonate membrane. The extrusion was performed 11 times through each membrane at 65° C.

Bacteria-mimicking liposomes: E. coli extract was dissolved in a mixture of chloroform and methanol (2:1 vol:vol) and evaporated using the first nitrogen stream, followed by 3 hours of vacuum pumping.

For LPS-pMPC interaction studies, an additional E. coli extract mixed with 32 ng/mL of LPS was prepared in an organic solvent. The lipid film was then hydrated with 5 mL calcein solution (35 mM, osmolarity=300 mOsm/kg) at 35° C. The resulting multilamellar vesicles (MLVs) suspension was sonicated for 15 minutes at 35° C., and subsequently downsized by extrusion (Lipex®, Northern Lipids Inc.) through 400 nm, 200 nm polycarbonate membranes. The extrusion was performed 11 times through each membrane at 35° C. Liposomes were then separated from an excess of free calcein by 48 hour—dialysis (Float-A-Lyzer®, Sigma-Aldrich™) against PBS.

The size and zeta-potential of the lipid nanoparticles were measured with a ZetaSizer Nano ZS (Malvern Instruments, UK) Dynamic Light Scattering (DLS) at 25° C. Triplicate measurements with a minimum of 10 runs were performed for each sample.

Calcein assay: Membrane fusion of surface-functionalized (either with PEG- or pMPC-) or bare liposomes with bacteria biofilm or with bacteria-mimicking liposomes was monitored by calcein dequenching methods as described elsewhere [Dutta et al. Bio-protocol 2020, 10 (14)].

Functionalized or bare liposomes were prepared using an extruder, where lipid film was hydrated with a solution of (CH₃COO)₂Mg containing 70 mM calcein. Osmolarity of (CH₃COO)₂Mg/Calcein mixture was adjusted to 320 mOsm/kg. The loaded vesicles were then separated from an excess of free calcein by Sephadex® G-25 pre-equilibrated with Na₂SO₄ followed by 24 hours—dialysis (Float-A-Lyzer®, Sigma-Aldrich™) against Na₂SO₄. The calcein-loaded liposomes (20 μL, 2 mM final concentration, shaken) were added to 150 p L of 24h-biofilm.

2. Bacteria-mimicking liposomes (E. coli total extract) were prepared using an extruder, where lipid film was hydrated with calcein solution (35 mM, osmolarity=300 mOsm/kg). 25 μL of bacteria-LUVs (concentration 1.7 mM) was added into 20 p L of 10 mM HSPC/cholesterol (40% mol/mol)/pMPC (5 kDa, 5% mol/mol) and 160 μL PBS (−/−Ca²⁺/Mg²⁺) or PBS (+/+Ca²⁺/Mg²⁺).

Continuous monitoring of calcein fluorescence (excitation 470 nm, emission 509 nm) was done at intervals of 15 minutes for a period of 24 hours (without shaking) at 37° C., using a ClarioStar microplate reader (BMG LABTECH GmbH, Germany). The final fluorescence intensity, which represents maximal fluorescence of free calcein was determined following the solubilization of vesicles with Triton X-100 (2% vol/vol) and compared to the value of calcein from solubilized liposomes in the biofilm- or bacteria-liposome free medium.

Giant unilamellar vesicles preparation (GUVs): Giant unilamellar vesicles (GUVs) of DPPC/pMPC (5 kDa, 5% mol/mol) or DPPC/PEG (5 kDa, 5% mol/mol) labeled with 0.1% (mol/mol) DiI dye were prepared using the polyvinyl alcohol (PVA) gel-assisted formation method as described by Weinberger et al. [Biophysical Journal 2013, 105 (1), 154-164].

Briefly, 200 mL of 5% (w/w) PVA solution was spread on a glass slide and dried for 30 minutes at 80° C. Once the PVA-coated substrate was prepared, 5 mL of lipid in chloroform/methanol (2:1) (1 mg/mL) was spread and placed under vacuum for 30 minutes to evaporate the organic solvent. Using a rubber gasket as a temporary chamber, the lipid film was hydrated with a PBS solution (318 mOsm/kg) and left incubating for 60 minutes at 50° C. After incubation, the GUVs were collected and transferred to microscopy glass.

Remote drug loading and efficiency determination: Active loading of SMX and SMX/EA was based on a modified approach of Clerc and Barenholz [Biochimica Et Biophysica Acta Bba-Biomembr 1995, 1240 (2), 257-265].

Freshly prepared lipid suspension was passed through a size-extrusion column, Sephadex® G-25, pre-equilibrated with Na₂SO₄ (pH of about 5.5, 320 mOsm/kg), which creates a proton/transmembrane gradient inside the lipid carrier. The low pH outside of the vesicles allows the drug to be uncharged, and therefore to freely diffuse across the lipid membrane towards the inside. The higher pH of the liposomes' lumen allows ionization of the drug, and favoring its accumulation. Liposomal sample was heated above phase transition of HSPC-lipid (65° C.), and a defined amount of SMX in DMSO (5% v/v) was added. Magnetic stirring allowed a homogeneous distribution of drug in sample. For single-drug-loaded liposomes sample was left for 90 minutes at 65° C.

For liposomes loaded with both SMX and EA: after 90 minutes of equilibration with SMX, EA was added at a concentration 1.1 mg/mL and the sample was equilibrated for another 45 minutes. Subsequently, samples were cooled rapidly in an ice bath and non-entrapped drugs were removed using size-extrusion Sephadex® G-25 column pre-equilibrated with BM2G or Na₂SO₄ (pH 7.0, ˜320 mOsm/kg). To ensure complete removal of residual DMSO (dimethyl sulfoxide), samples were overnight dialyzed (MWCO 50 kDa, 4° C.) against BM2G or Na₂SO₄ (pH 7.0, ˜320 mOsm/kg).

Intra-liposomal drug content measurements: After loading, 20 μL of the sample was diluted with 1 mL of ethanol (for EA determination, 10 mM Borax was added to the solution) and placed in a bath sonicator for 10 minutes.

The concentration of SMX and EA inside liposomes was determined by an ultraviolet spectrophotometer (Cary 100 Bio, Varian Inc., USA) at wavelengths 264 nm and 367 nm, respectively.

The loading efficiency was calculated as a mole ratio between loaded drug and HSPC lipid. Concentration of HSPC lipid after loading procedure was determined based on NanoSight (NTA) results.

NTA measurements: Samples were diluted with PBS to a final volume of 1 mL. Optimal dilution for each sample was found by pre-testing the sample until ideal particle-per-frame value (20-100 particles/frame) was obtained. For each measurement, five 1-minute videos were captured at 25° C., with at least 300 μL displacement between each video. The number of completed tracks in NTA (Malvern NanoSight NS300, Malvern, UK) measurements was always greater than the proposed minimum of 1000 in order to minimize data skewing based on single large particles.

Determination of drug release profile: Drug-loaded LUVs sample were placed in a dialysis tubing with a molecular cutoff of 50 kDa and sample was dialyzed against PBS buffer (about 320 mOsm/kg, pH 7.4). Samples were incubated at 37° C. and at defined time points, 20 p L of each sample were collected and analyzed for drug content as described above, and PBS outside the dialysis bag was exchanged.

Cryo-TEM measurements: A Vitrobot plunger system Mark IV (FEI, USA), was used to prepare the samples for cryo-TEM. Humidity was kept close to 80% for all experiments and the temperature was set at 24° C. 3 μL of the liposomal suspension (concentration, 1.5 mg/mL) were placed onto a holey carbon grid (C-flat 2/2 200 mesh) (Electron Microscopy Sciences, Hatfield, PA, USA) which was rendered hydrophilic via glow discharge (30 seconds, 25 mA) (PELCO easiGlow™, Redding, CA, USA). Excess sample was removed by blotting (3 seconds) with filter paper, and the sample grid was vitrified by rapid plunging into liquid ethane. The sample imaging was conducted on a Talos Arctica G3 TEM/STEM (FEI, USA) cryo-electron microscope equipped with an OneView camera (Gatan Inc.) at an accelerating voltage of 200 kV. Images were acquired in low-dose mode using the SerialEM software (FEI, USA) to avoid radiation damage to the samples at 73000× magnification with a defocus value in the range of [−2 μm; −4 μm].

General bacterial growth conditions: P. aeruginosa UCBPP-PA14 or P. aeruginosa strain LESB58 (an epidemic strain isolated from chronically infected CF patients) precultures cultures were inoculated at OD600 0.05 from an overnight culture, and growth was carried out at 37° C. with shaking for 4 hours, in Luria-Bertani broth (LB) for PA14 and Tryptic Soy Broth medium (TSB) for LESB58 until mid-exponential phase (OD at 600 nm is about 0.5).

Then, for the formation of a biofilm, bacteria were diluted 1:100 in BM2G minimal medium (62 mM potassium phosphate buffer, pH 7, 7 mM (NH₄)₂SO₄, 2 mM MgSO₄, 10 μM FeSO₄, 0.4% (wt/vol) glucose, 1% monosodium glutamate) and grew in 96 wells plate at 37° C. without shaking for 24 hours [Winstanley et al. Genome Res 2009, 19 (1), 12-23].

For CFU, bacteria were grown in BM2 at 37° C. with shaking for 16 hours every 2 hours, 100 μl of bacteria were serially diluted in sterile phosphate-buffered saline (PBS) and the dilutions were plated on two LB agar medium Total bacterial counts obtained and compare to Resazurin assay.

Single cells fluorescence microscopy: Liposomal fusion with bacteria cells was visualized using single cell fluorescence microscopy. In brief, large unilamellar vesicles (LUVs) were prepared using an extruder, the lipid film was hydrated with a solution of magnesium acetate containing 70 mM calcein. The osmolarity of the magnesium acetate/calcein mixture was adjusted to 320 mOsm/kg. The calcein-loaded vesicles were then separated from an excess of free calcein by Sephadex® G-25 pre-equilibrated with BM2G medium (about 320 mOsm/kg) followed by overnight dialysis (MWCO 50 kDa) against BM2G.

PA14 cells were grown in BM2G medium at 37° C., 200 rpm. When growth reached an OD600 of 0.3, bacteria were incubated with liposomes loaded with calcein (0.03 mM final liposomal concentration) for 24 hours at 37° C. At t=4 hours and t=24 hours, 500 μL of the samples were centrifuged at 8000 g for 2 minutes at 25° C. and re-suspended in 10 μL PBS. Samples were visualized using an Axioplan2 microscope (ZEISS, Germany) equipped with ORCA Flash 4.0 camera (Hamamatsu Photonics). System control and image processing were performed using Zen version 2.6 (ZEISS, Germany).

Super-resolution microscopy (d-STORM): Three-dimensional d-STORM imaging was performed using Vutara SR352 microscope (Bruker®, USA) based on single-molecule localization biplane technology. Images were recorded using 1.3 NA 60×silicon oil immersion objective (Olympus) and Hamamatsu ORCA-Flash 4v2 camera with a frame rate at 50 Hz. Bacteria were incubated with liposomes labeled with DiR (0.5% mol/mol) dye for 4 hours, then washed three with PBS. Next, bacteria were stained with 1 mg/ml membrane stain FM1-43 (Thermo Fisher Scientific™ T35356), washed three times with PBS were, centrifuged at 5000 rpm for 2 minutes at 25° C., and re-suspended. Then, fixed with 2.8% formaldehyde (FA), 0.04% glutaraldehyde-formaldehyde (GA) for 15 min, washed three times and were centrifuged at 5000 rpm for 2 minutes at 25° C. and re-suspended in 10 μL PBS. Bacteria were dropped on a poly-L-lysine (Sigma-Aldrich™ P8920) coated MatTek plate (P35G-1.5-7-C), and imaging was performed in the presence of an imaging buffer (7 μM glucose oxidase (Sigma-Aldrich™), 56 nM catalase (Sigma-Aldrich™), 2 mM cysteamine (Sigma-Aldrich™), 50 mM Tris, 10 mM NaCl, 10% glucose, pH 8). Subsequently, STORM images of liposomes were recorded using 640 nm excitation laser (maximal excitation of 6 kW/cm²) with a collection of 3000 frames. Data were analyzed with the Vutara SRX 7.0.00rc24 software.

Determination of biofilm eradication by a combination of MBEC™ and resazurin assays: PA14-biofilms were formed using MBEC™ assay system (Innovotech, USA) as previously described with slight modifications [Ceri et al. J Clin Microbiol 1999, 37 (6), 1771-1776].

Briefly, bacterial suspension was adjusted to OD of about 0.5. The biofilm device was inoculated by adding 150 μL of the inoculum into the wells of the 96 peg-lids on which the biofilm cells could build up. Respective negative controls (BM2G medium only) wells were also prepared. The pegs were incubated in a humidified incubator for 24 hours at 37° C. to allow biofilm formation on the purpose-designed pegs. Once biofilm was fully formed, the peg-lid was washed by moving for 10 seconds to a new 96 well containing 160 μL of PBS in each well. The washing procedure was performed twice.

Subsequently, the peg lid was then transferred into a ‘challenge 96-well microtiter plate’ containing 170 μL of antimicrobial treatment i.e. free SMX, or liposomes loaded with antimicrobial treatment. The peg lid was incubated for 4 hours at 37° C. After incubation, the peg lid was washed with 180 μL of PBS, and once more the biofilm was exposed to liposomal treatment. Finally, the PEG lid was removed and washed twice with PBS as previously. PEG-lid wad transferred into the recovery plate containing 190 μL of BM2G in each well and sonicated using a bath sonicator for 10 minutes at room temperature.

For cell viability, cells that were removed from the MBEC lid by sonication in BM2G medium containing 2 μL of resazurin stock solution (1 mg/mL) in 96 wells plate and fluorescence (λ_{Ex} 530 nm and 560-650 nm emission spectra) was using ClarioStar microplate reader (BMG LABTECH GmbH, Germany). Fluorescence signal was measured with 15 minutes intervals for 15 hours at 37° C. with 100 rpm shaking mode. In the wells where no dye was added the absorbance at 600 nm was measured.

Determination of bacterial viability in biofilms by LIVE/DEAD™ staining: PA14-biofilms were developed on 8 Well p-Slide (ibidi GmbH, Germany). 150 μL of the inoculum was incubated in a humidified incubator for 24 hours at 37° C. without shaking. After 24 hours, the fully formed biofilm was gently washed twice with PBS. 160 μL of liposomal suspension or BM2G medium was added to each well, and subsequently, the samples were incubated at 37° C. for 4 hours. Each well was then washed with PBS, and a second dose (170 μL) of antibacterial treatment was applied for 4 hours with incubation at 37° C. Subsequently, biofilm was washed with 180 μL of PBS buffer, and bacteria cells were stained using a BacLight™ Bacterial Viability Kit@ (Thermo Fisher Scientific™, USA). Live bacterial cells were stained with SYTO™ 9 dye (ex: 486/em: 501) and dead cells were stained with propidium iodide (PI) dye (λ_{Ex}: 535/λ_{Em}: 617), with dyes mixed in 1:1 ratio. 170 μL of Syto9/PT in BM2G medium was added to wells and incubated for 20 minutes at room temperature. Later, wells were washed twice with PBS, and images of biofilm were acquired using confocal fluorescence microscopy. Cell viability quantification was performed using ImageJ software v1.52i (NIH, USA). Confocal pictures were acquired using a confocal laser scanning fluorescence microscope LSM700 (ZEISS, Germany). All images were acquired using a 40×oil immersion objective, with a 0.3 μm optical slice step for z scanning. Images were recorded in brightfield mode and in confocal mode using 488 excitation and 561 excitation laser channels. Picture analysis was performed using ImageJ software v1.52i (NIH, USA). For comparative analysis, all parameters during image acquisition were kept constant throughout each experiment.

Paraffin-embedded thin sectioning and LIVE/DEAD staining for fluorescence imaging: Thin sectioning assays were performed as described, for example, in Cornell et al. J Vis Exp 2018, No. 133.

Briefly, 5 μl of subcultures were spotted onto 1% agar plates containing a two-layered growth medium (1% tryptone, grown in the dark at 25° C. with >90% humidity. After three days, 10 μl liposomes were added on the top of colony and incubated for 4 hours in the dark at room temperature. Colonies were stained using 20 μl of a BacLight™ Bacterial Viability Kit@ (Thermo Fisher Scientific™, USA) for 15 minutes. Subsequently, colonies were covered with an agar layer, and sandwiched colonies were lifted from the bottom layer, washed for 10 minutes in PBS (pH 7.4) at room temperature in the dark, and fixed in 4% paraformaldehyde with 50 mM L-lysine hydrochloride in PBS overnight at room temperature in the dark. Fixed colonies were washed twice in PBS and dehydrated and paraffin-embedded through a series of ethanol washes (70%, 95% (x3), 100% (x2), ethanol/X-TRA SOLVE (MEDITE® 41-5213-00) (50%/50%) for 1 hour. Then, Colonies were paraffin-embedded via three 60-minutes incubations in X-TRA SOLVE 530 at 57° C. in Fully Enclosed Tissue Processor (Leica® ASP300S). Then, colonies were allowed blocked overnight at 4° C. in Paraplast Plus Paraffin Wax (Leica Biosystems 39602004). Tissue processing was performed using a Tissue Embedding Medium Surgipath Paraplast Plus Paraffin White Solid. Trimmed blocks were taken in 1 mm deep from the center of the biofilm in the block, sectioned in 10-μm-thick sections, 5 μm angels perpendicular to the plane of the colony using microtome (Leica RM2265 Microtome), and collected onto slides. Slides were air-dried overnight, heat-fixed on a hotplate for overnight at 37° C., and rehydrated. Rehydrated colonies were immediately mounted in buffer (Leica Biosystems—EG 1160) and overlaid with a coverslip.

Confocal pictures were acquired using a confocal laser scanning fluorescence microscope LSM700 (ZEISS, Germany).

All images were acquired using a 40×oil immersion objective, and individual fields of view were subsequently stitched together to form the entire section. Images were recorded in brightfield mode and in confocal mode using a 488 excitation and 561 excitation laser channels. Picture analysis was performed using ImageJ software v1.52i (NIH, USA). For comparative analysis, all parameters during image acquisition were kept constant throughout each experiment.

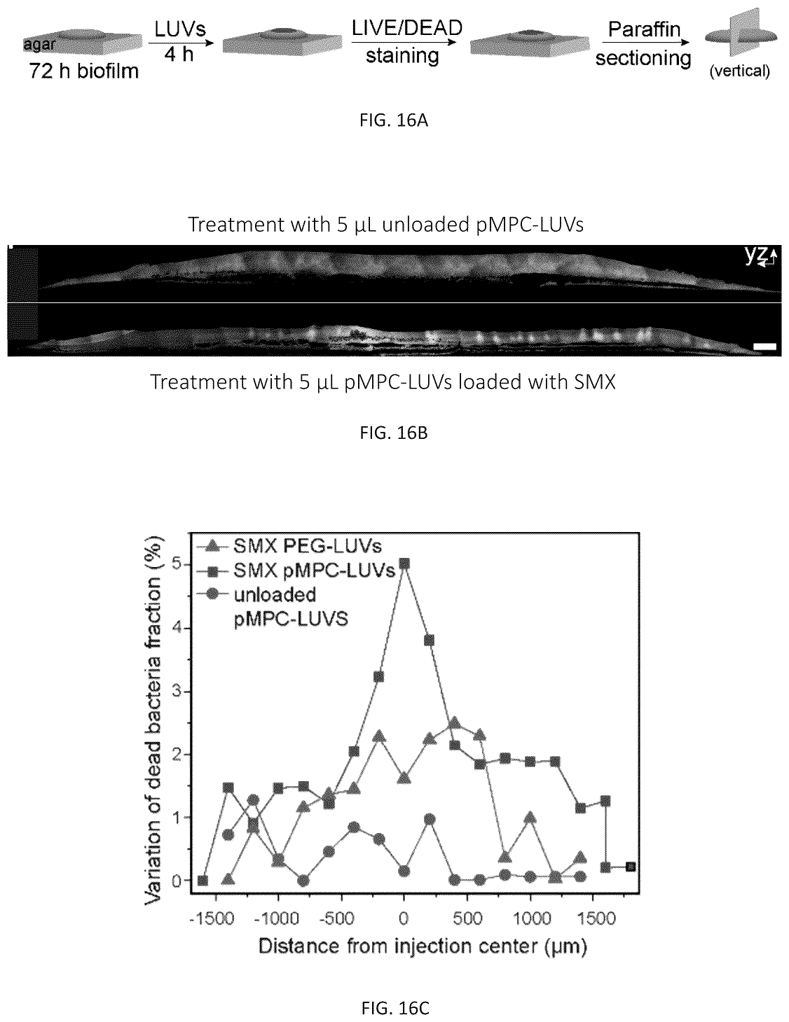

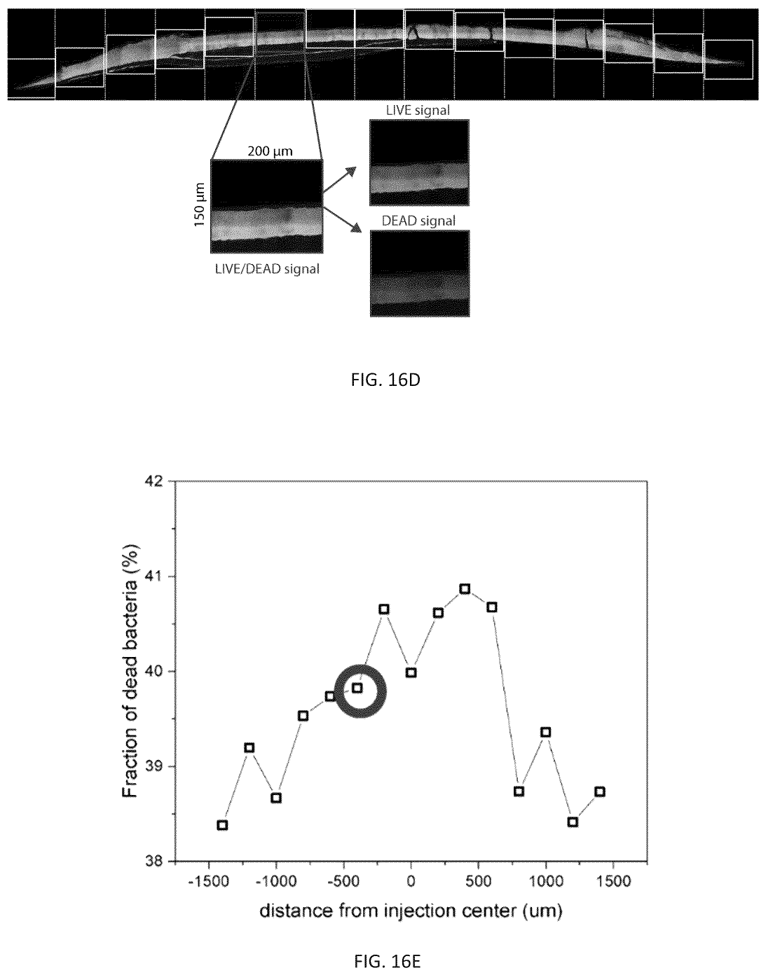

Data analysis: Images of bacterial slices displaying the full-length sample both in green and red channels were contoured to isolate only the biofilm section and remove the majority of the fluorescent background signal using Fiji. The resulting composite images with a removed background were then segmented with a 200 μm wide and 150-200 μm high region of interest to encompass the slice at its thickest point. For each segment, the average intensity of the two channels (green—LIVE signal, red—DEAD signal) was measured, and the fraction of dead bacteria was quantified as the ratio of the red intensity over the sum of the two channels. To compare different biofilm slices and different treatments, each spatial profile of dead bacteria fraction was normalized as variation from its lowest value (see, FIGS. 16D-E).

Evaluation of cytotoxicity: Liposomes cytotoxicity was determined by the production of the yellow formazan product upon cleavage of XTT by mitochondrial dehydrogenases in viable HEK293 cells (human embryonic kidney). The cells were seeded onto 96-well plates (4×10⁴ cells/well) in DMEM media. When the confluent state was reached (usually after 24 hours), 50 μL of pMPC-, PEG- liposomes and SMX (1.5 mg/mL) or SMX (0.7 mg/mL)/EA (1.5 mg/mL) mixture in PBS solutions were then added. After 24 hour incubation period, the cells were incubated with 50 μL of XTT solution composed for 3 hours. Absorbance values were later measured with a multiwell-plate reader (Cary 100 Bio, Varian Inc., USA) at a wavelength of 450 nm. Background absorbance was measured at 620 nm and subtracted from the 450 nm measurement. The experiments were repeated at least three times, and six replicates were prepared for each liposomes concentration tested in every experiment. A solution in DMEM culture medium was used as a positive control.

The potential toxic effect of the different liposomal formulations tested was expressed as a viability percentage calculated using the following equation (1):

$$\% Viability = 100 - [ ( ODtest / ODc ) \times 100 \% ] \qquad ( 1 )$$

Where ODtest was the optical density of those wells treated with the liposomes solutions, and ODc was the optical density of those wells treated with supplement-free DMEM media.

Pyocyanin quantitation assay: Pseudomonas aeruginosa biofilms were grown on 24-well plate. 1 mL of the inoculum was grown in a humidified incubator for 24 hours at 37° C. without shaking. After 24 hours, the fully formed biofilm was gently washed two times with PBS. 1 mL of liposomal suspension or BM2G medium was added to each well and subsequently the samples were incubated at 37° C. for 4 hours. Each well was then washed with PBS and second does (1 mL) of antibacterial treatment was applied for 4 hours with incubation at 37° C. Subsequently 24-well plate was sonicated at room temperature for 5 minutes. The collected bacteria suspension were centrifuged at 4500 rpm for 20 minutes. Obtained supernate was filtrated through 0.22 μm syringe filter. 0.5 mL of supernate was mixed with 0.17 mL of chloroform and shaken vigorously.

Subsequently, samples were centrifuged at 4500 RPM (round per minute) for 10 minutes. The organic phase was transfer to new tubes and mixed with 0.12 mL of 0.2 M HCl and shaken vigorously. The Samples were again centrifuged at 4500 rpm after which water phase was collected and pyocyanin was quantified using HPLC analysis.

Pyocyanin analysis by HPLC: Samples and pyocyanin standard solutions were prepared in 0.2 M HCl, supplemented with 1,5-Naphthalenediamine (0.1 mg/mL) as internal standard and filtered through 0.2 mm PTFE filters (StarTech®, CA) prior to HPLC analysis. Sample solutions were kept at 4° C. prior to injection and were separated by reverse-phase chromatography on a Gemini C18 (4.6×150 mm, 5 mm) column (Phenomenex®, USA). Chromatography was performed on a Prominence UFLC LC-20AD system (Shimadzu®, Japan) consisting of a SIL-20AC autosampler (Shimadzu®, Japan), CTO-20AC column oven (Shimadzu®, Japan) and a SPD-M20A diode array detector (Shimadzu®, Japan). Elution was done using an isocratic gradient of 70% solvent A (0.04% TFA in water) and 30% solvent B (10% water and 0.04% TFA in acetonitrile) at a flow rate of 1 mL per minute for 5 minutes at 25° C., while monitoring at 388 nm. Data analysis was performed using LabSolutions version 5.97 (Shimadzu®, Japan).

Stability of loaded liposomes: Liposomes with encapsulated drug were store at 4° C., and at define time points, liposomal suspension was dialyzed (dialysis bag with 50 kDa cutoff) against Na₂SO₄ (pH 7.0, about 320 mOsm/kg). Amount of remaining drug encapsulated in liposomes was determined using ultraviolet spectrophotometer (Cary 100 Bio, Varian Inc., USA).

Statistical analysis: All statistical assays performed were analyzed using Analysis of Variance (ANOVA) and then Tukey's test using OriginJ software v1.52i (NIH, USA). P-values<0.05 were considered statistically significant.

### Example 1

### Design and Preparation

As discussed herein, biofilms are not easily targeted by existing antibiotic-delivery systems due to difficulties in penetrating the films physical barriers and overcoming drug resistances. This results in currently available treatments for biofilm infections being poorly efficient and very side-effects prone [Attinger and Wolcott, 2012, supra].

In order to design a liposomal nanocarrier that would overcome the deficiencies of currently known carriers and would promote penetration to, e.g., negatively-charged bacterial membrane and/or biofilms, the functionalizations of liposomes were modified and a positively charged compound was introduced.

For this purpose, exemplary moieties consisting of lipid-conjugated phosphocholinated polymers (poly [2-(methacryloyloxy)ethyl phosphorylcholine], pMPC) [Lin et al. Langmuir 2019, 35 (18), 6048-6054; and Lin et al. J Mater Chem B 2022, 10, 2820] were inserted into the liposomal membranes.

Such moieties are also highly hydrated [Chen et al. Science 2009, 323 (5922), 1698-1701], and have demonstrated suppression of non-specific protein adsorption [Zhang et al. Nano Res 2016, 9 (8), 2424-2432; and Adler et al. J Mater Chem B 2021, 10, 2512], while pMPC coating of either chitosan nanoparticles or DNase resulted in their markedly higher diffusion within the biofilms matrix [Cao et al. 2019, supra; and Liu et al. 2020, supra].

Without being bound to any particular theory, it was assumed that the highly-dipolar zwitterionic nature of the phosphocholine-like structure of the MPC monomers would result in interactions with the heterogeneously-charged bacterial cells. Such interactions may be stronger than those of stabilizing moieties such as PEG which are only weakly-polar.

It was assessed that the incorporation of a positively charged lipid, would result in an offset of the negative charge associated with the DSPE, as illustrated in the hydrophilic head group on FIG. 1. By this, it was assumed that the presence of an exemplary positively charged lipidic molecule such as stearylamine (SA) would promote interaction with bacterial membrane (e.g., biofilm-forming bacteria).

As exemplary liposomes, large unilamellar vesicles (LUVs) composed of saturated hydrogenated soybean phosphatidylcholine (HSPC) and containing 5% (mol/mol) pMPC-conjugated distearylphosphorylethanolamine (DSPE) (M_{pMPC}=5 kDa) were prepared as previously described [Lin et al. 2019, supra; and as described hereinabove, see Liposome preparation (LUVs), size and zeta-potential characterization]. In order to enhance mechanical flexibility and cargo-retention, liposomes were doped with 40% cholesterol. 5% (mol/mol) stearylamine (SA) was added to the final composition as an exemplary positively charged agent.

The liposomes were extruded through a 200 nm membrane to provide exemplary pMPC-functionalized (here, also pMPCylated) LUVs, as schematically illustrated in FIG. 1. PEG-functionalized (PEGylated) LUVs incorporated with PEG-conjugated DSPE (M_{PEG}=5 kDa) instead of the pMPC moieties were prepared in a similar manner.

### Example 2

### Characterization

Table 1 hereinbelow shows the Zeta potential and hydrodynamic diameter of pMPC-LUVs and PEG-LUVs.

TABLE 1

| | Unloaded | | |
|---|---|---|---|
| | Diameter | | ζ-potential |
| Sample | (nm) | PDI | (mV) |
| HSPC (50%)/cholesterol (40%)/ | 179.7 | 0.04 | −4.24 ± 0.02 |
| stearylamine (5%)/pMPC (5 kDa, 5%) | | | |
| HSPC (50%)/cholesterol (40%)/ | 165.5 | 0.04 | −1.84 ± 0.08 |
| stearylamine (5%)/PEG (5 kDa, 5%) | | | |

Values are shown as averages over 3 samples with 10 runs each.

The colloidal stability of different LUVs under storage at 4° C. was examined, and the results are presented in FIG. 2A. As can be seen, the pMPCylated LUVs have long-term stability against aggregation, showing uniform size distribution peaking at about 180 nm diameter and with low, constant polydispersity for up to 24 weeks (PDI<0.1).

Non-functionalized liposomes were also examined for their stability. For this purpose, liposomes composed of HSPC (60%)/cholesterol (40%); and HSPC (55%)/cholesterol (40%)/stearylamine (5%), were examined, and the results are presented in FIGS. 2B and 2C, respectively. A photograph of the obtained liposomes is presented in FIG. 2D.

As can be seen, non-functionalized vesicles exhibit poor colloidal stability and rapid aggregation within approximately 3 days. Data also indicate that stearylamine provides the LUVs with an increased stability over time, even in non-functionalized liposomes.

The effect of the exemplary PEGylated and pMPCylated LUVs with HEK293 cells was examined, and the results are presented in FIG. 3. As can be seen, functionalization and the incorporation of the stearylamine did not compromise the biocompatibility of the vesicles, and even improved cell viability in comparison to a free drug (SMX).

### Example 3

### Divalent Ion-Mediated Adhesion of Functionalized LUVs to Bacterial Membrane

Calcium and other divalent ions are highly abundant at the outer lipopolysaccharide (LPS)-exposing membrane surfaces of gram-negative bacteria, where they play a crucial structural role [Clifton et al. Langmuir 2015, 31 (1), 404-412; Lam et al. Soft Matter 2014, 10 (38), 7528-7544; Silhavy et al. Csh Perspect Biol 2010, 2 (5), a000414; and Holst et al. Part Microb Glycolipids Glycoproteins Glycopolymers 2010, 1-13].

In order to examine the affinity between the phosphocholine group-comprising pMPCylated LUVs and the LPS of the bacterial surface, the effect of Ca⁺², as an exemplary multivalent ion, was directly examined on the exemplary pMPCylated LUVs and comparable PEGylated LUVs.

Cryo-TEM was used to image pMPCylated and PEGylated LUVs following 1-hour incubation with increasing concentrations of Ca(Ac)₂. The cryo-TEM images are presented in FIGS. 4A-C, and its quantification is presented in FIG. 4D.

As shown in FIG. 4D, in the presence of 10 mM or 40 mM Ca(Ac)₂, pMPC-LUVs display adhesion, with 60% and 98% incidence, respectively. The arrows in FIGS. 4A-B show that the interaction between vesicles changes from weak adhesion at 10 mM to a highly-flattened contact region at 40 mM, indicating strong adhesion.

In sharp contrast, PEG-LUVs incubated at 40 mM Ca(Ac)₂ do not adhere at all, and have stochastically-distributed separations between adjacent vesicles, as can be seen in FIGS. 4C-D.

These results demonstrate the role of Ca⁺² in bridging pMPC (but not PEG) moieties on neighboring vesicles.

As seen in FIG. 4D, the gaps between adhering outer membranes are about 4 nm for both Ca⁺² concentrations, which is in consistence with two interdigitating opposing layers of 5 kDa pMPC-chains bridged by calcium [Kobayashi et al. Soft Matter 2013, 9 (21), 5138-5148].

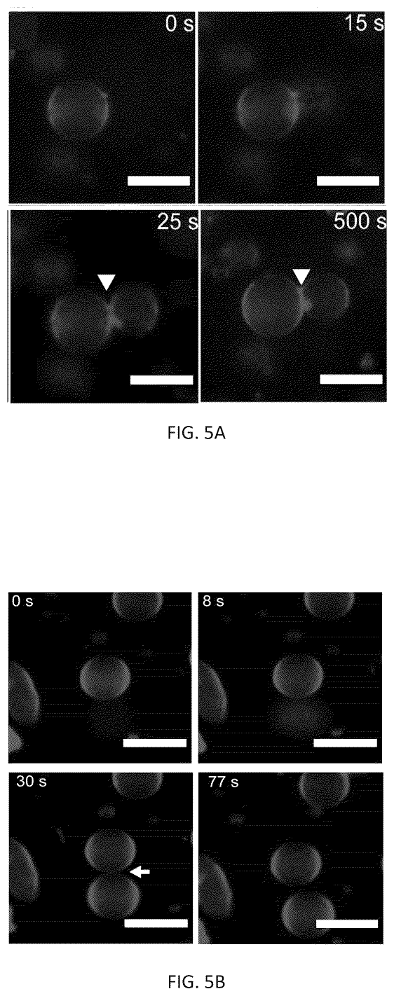

In order to visualize the rate of the adhesion process, live imaging microscopy on pMPCylated giant unilamellar vesicles (GUVs) was performed in the presence of 40 mM Ca(Ac)₂ and in the presence of HEPES-glucose buffer, and images are presented in FIG. 5A-B, respectively.

As can be seen, the adhesion process in the presence of Ca(Ac)₂ is rapid, with GUVs display long-lasting adhesion occurring within 25 seconds of contact of the opposing membranes, whereas in the absence of divalent ions conditions, GUVs do not stably adhere to each other, even upon stochastic contact.

To further corroborate the role of calcium ions in pMPC interaction with bacterial membranes, calcein release of LUVs composed of bacteria phospholipids extract (either with membrane-incorporated LPS or LPS-free) incubated with pMPC-LUVs was examined under differing calcium concentration.

The data, presented in FIGS. 6A-B, show that bacteria-mimicking liposomes containing LPS, when mixed with pMPC-LUVs, display a significant increase in calcein fluorescence in the presence of a low calcium concentration (0.72 mM), reaching 18% content mixing within 5 hours.

Contrariwise, incubation in calcium-free conditions causes only a negligible increase in calcein fluorescence of bacterial liposomes (about 2.3%) upon incubation with pMPC-LUVs. Similarly, LPS-free bacteria-mimicking liposomes show moderate content mixing (about 7%) upon interaction with pMPC-liposomes under calcium-conditions, while in calcium-free milieu only minimal calcein release occurs.

Overall, these results suggest that the exemplary pMPC-functionalized liposomes would display enhanced interaction with the LPS- and Ca⁺²— containing bacterial membranes.

### Example 4

### Functionalized Liposomes Attachment to Bacterial Membranes

Pseudomonas aeruginosa (PA) biofilms was used herein as a model for a gram-negative bacterial infection.

Super-resolution microscopy (STORM) at a single-cell level was used to examine the distribution of pMPCylated liposomes following a 4-hour incubation period with P. aeruginosa cells.

The data on FIGS. 7A-C reveal that in comparison with PEGylated LUVs, about 3.3-fold more pMPCylated LUVs bind to the outer bacterial membranes.

As can be seen in FIG. 7A, a significant number of pMPCylated vesicles are also observed in the intercellular space. As cells were thoroughly washed following their incubation with the liposomes and only then placed on an imaging glass, these LUVs are not attached to the glass substrate.

Without being bound do any particular theory, it is assumed that the LUVs are adhering either to the residual extracellular matrix and/or to filamentous structures extending from the PA14 cells [Al-Wrafy et al. Postgpy Higieny Medycyny Doświadczalnej 2017, 71 (1), 78-91; and Touhami et al. J Bacteriol 2005, 188 (2), 370-377].

### Example 5

Delivery of Cargo into Bacterial Cytosol

The delivery efficiency of pMPCylation in releasing liposomal cargo into the bacteria cytosol was determined with single-cell fluorescence microscopy imaging of bacteria incubated with calcein-loaded liposomes at t=4 hours and at t=24 hours as shown in FIGS. 8A-C, following thorough washing of the cells after incubation.

As can be seen in FIG. 8A, in agreement with the STORM images, the 4-hour incubation period reveals a higher number of pMPC-functionalized liposomes compared to PEGylated LUVs, with small lipid aggregates visible both near the bacteria membrane and in the intercellular space.

Following a 24-hour incubation period, as shown in FIGS. 8B-C, pMPCylated LUVs released their calcein cargo into a much larger fraction of bacteria (70%) compared to PEGylated liposomes (35%), and moreover with a brighter calcein signal per cell.

Interestingly, after 24 hours, pMPC liposomes were not observed in the intercellular spaces, suggesting that most of the added vesicles were internalized by cells.

The kinetics of the observed payload delivery was studied via calcein dequenching assay as described herein, and is shown in FIGS. 8D-E for two different strains of P. aeruginosa, UCBPP-PA14 and LESB58. The improved efficiency of pMPCylated liposomes compared to PEGylated liposomes is demonstrated, with nearly 100% of the total dye being released by the pMPCylated liposomes over 17 hours as opposed to about 40% for the PEGylated liposomes in the PA14 strain.

In order to assess the effect of liposome diameter and stearylamine presence on calcein release profiles upon interaction between different liposomes and PA14 cells, the release of calcein in biofilm was compared between LUVs containing 5% mol of cationic lipid stearylamine (SA) and LUVs with no positive charge in the membranes, as seen in FIGS. 9A-D.

The data demonstrate that a rapid cargo release requires the presence of positively charged lipids (e.g., stearylamine), as FIGS. 9C-D show that incubation with LUVs which do not comprise stearylamine still shows calcein release, but with a characteristic lag time of about 400 minutes.

The 2.5-fold difference in cargo release in comparison with FIG. 8D arises from the different interactions between the respective liposomes and the biofilm, as incubation of liposomes with bacteria culture media (BM2G) alone shows no changes in calcein intensity (FIG. 8D and the blue circles in FIGS. 9A-D) or liposome size. The effect of BM2G medium on the hydrodynamic diameter of pMPC-LUVs and PEG-LUVs (extruded through 200 nm pore membrane) is summarized in Table 2 hereinbelow.

TABLE 2

| | Na₂SO₄ | | BM2G | |
|---|---|---|---|---|
| | Diameter | | Diameter | |
| Sample | (nm) | PDI | (nm) | PDI |
| HSPC (50%)/cholesterol (40%)/ | 189.8 | 0.08 | 185.0 | 0.10 |
| stearylamine (5%)/pMPC (5 kDa, 5%) | | | | |
| HSPC (50%)/cholesterol (40%)/ | 161.4 | 0.08 | 159.3 | 0.08 |
| stearylamine (5%)/PEG (5 kDa, 5%) | | | | |

Values are shown as averages over 3 samples with 10 runs each, and the standard deviation is used as an error estimate.

Moreover, pMPCylated vesicles undergo faster interaction and cargo release than non-functionalized liposomes; while both conditions achieve full delivery within 17 hours, the non-functionalized LUVs show a 4 hours lag time prior to any calcein release. Additionally, as FIGS. 2B-D show, such non-functionalized vesicles are unstable and aggregate within 3 days.

To examine the generality of these observations, these measurements were repeated in a P. aeruginosa clinical strain, LESB58, a hypervirulent human cystic fibrosis isolate [Kukavica-Ibrulj et al. J Bacteriol 2007, 190 (8), 2804-2813].

As can be seen in FIG. 8E, while the overall release efficiency in this specific strain is lower for both functionalizations compared to PA14, with 25% release of the total calcein for pMPC-LUVs, the fold-differences are even larger, with pMPCylated vesicles having about 5-fold higher cargo release into the LESB58 biofilm compared to PEGylated ones.

The variation in total cargo release (100% for PA14 vs. 25% for LESB58 following 20 hours) may be due to differences in lipid [Ma et al. Int J Nanomed 2013, Volume 8 (1), 2351-2360; and Fillion et al. Biochimica Et Biophysica Acta Bba-Biomembr 2001, 1515 (1), 44-54]and biomolecules composition [Drulis-Kawa et al. Int J Pharmaceut 2009, 367 (1-2), 211-219]at the different bacterial surfaces.

Based on the results obtained using the combination of cryo-TEM, confocal microscopy, stochastic optical reconstruction microscopy (STORM), and fluorimetry (FIGS. 4A-D, FIGS. 5A-B, FIGS. 7A-C and FIGS. 8A-C, respectively), a two-step mechanism was suggested for the interaction between pMPC-stabilized liposomes and bacterial membranes and is schematically illustrated in FIG. 10.

In the first stage (1), increasing overlap of the pMPC-moieties with the surface-exposed negatively-charged LPS at the bacterial outer membrane is energetically favored by their Ca⁺²-mediated interactions. Maximizing this overlap results in the pMPCylated liposomes being ‘pulled in’ towards the cells, and to proximity-driven fusion between the liposome and cell lipid membranes, which is the second stage.

Without being bound to any particular theory, this fusion is attributed to the charge -charge interactions between the positively-charged stearylamine with the negatively-charged bacterial membrane, as previously reported [Drulis-Kawa et al., 2009, supra]. While the presence of negatively-charged LPS at the surface of the bacteria (e.g., gram-negative bacteria) target cells suggests this specific bridging configuration in the present study, similar divalent-ion mediated interactions could be expected between the dipolar MPC monomers and different negatively-charged groups also present at other cell surfaces [Nishino et al. Plos One 2020, 15 (7), e0236373].

In the next steps, (2) and (3), functionalization with pMPC promotes liposome-bacteria adhesion via divalent-ions-induced LPS bridging (which is not the case with PEG-LUVs). The liposome-bacteria adhesion leads to a higher fusion rate [Ma et al. Int J Nanomed 2016, 11, 4025-4036], with subsequent release of the vesicles cargo into the bacterial cytosol, as measured via single-cell microscopy and calcein de-quenching technique.

### Example 6

Multiple Drug-Loaded liposomes

In order to examine the ability of functionalized liposomes to serve as carriers for multiple cargo (e.g., multiple drugs for, e.g., combination therapy), liposomes were loaded either with the exemplary antimicrobial agents sulfamethoxazole (SMX) alone, or co-loaded SMX with the performance-enhancing phytochemical ellagic acid (EA) [Ejim et al. Nat Chem Biol 2011, 7 (6), 348-350].

Ellagic acid was previously shown to downregulate gene expression in bacteria [Huber et al. Zeitschrift Für Naturforschung C 2003, 58 (11-12), 879-884; and Weidner-Wells et al. Bioorg Med Chem Lett 1998, 8 (1), 97-100] and suppress production of pyocyanin (a virulence factor with several physiological roles in P. aeruginosa biofilms) in P. aeruginosa strains [Yang et al. Front Microbiol 2018, 8, 2640], resulting in enhanced cell sensitivity towards antimicrobial agents, as previously demonstrated for SMX [Jayaraman et al. Int J Biol Sci 2010, 6 (6), 556-568].

The chemical and physical properties of sulfamethoxazole (SMX) and ellagic acid (EA) are presented on Table 3 hereinbelow.

TABLE 3

| | Mw | Water solubility | | |
|---|---|---|---|---|
| | [g/mol] | [mg/mL] | LogP | pKₐ |
| Sulfamethoxazole (SMX) | 253.3 | 0.46-0.61 | 0.7-0.89 | 5.7 |
| Ellagic acid (EA) | 302.2 | 0.82 | 1.59-2.32 | 5.8 |

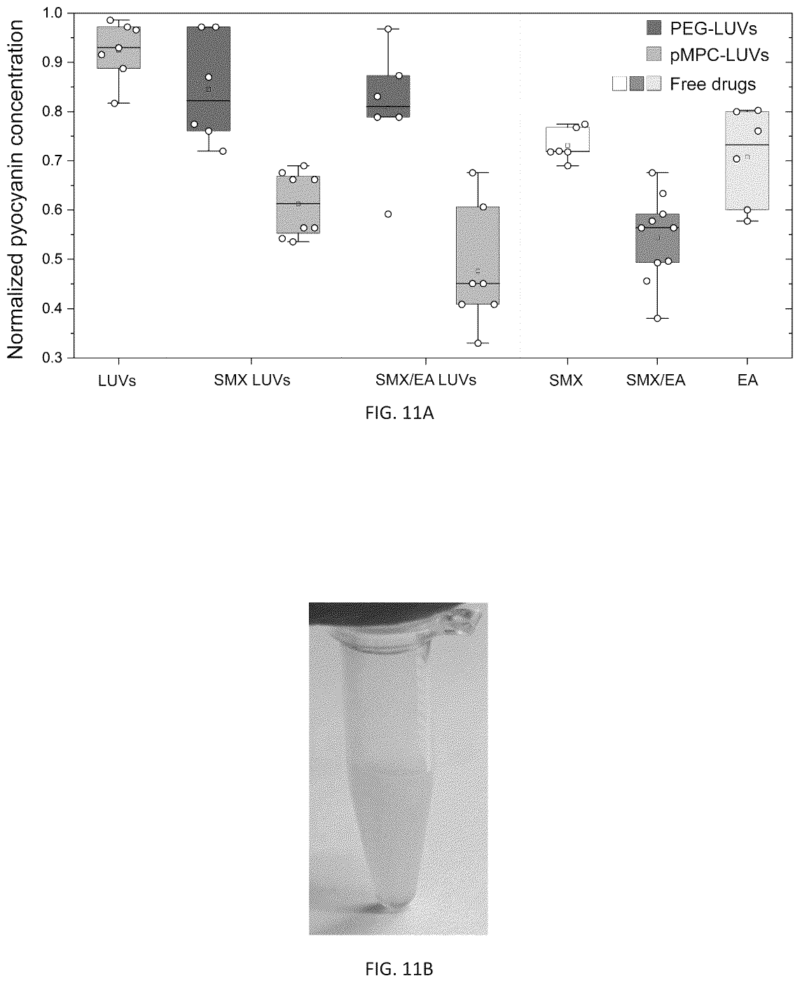

The effect of SMX and SMX/EA on pyocyanin production in P. aeruginosa was examined using PEG-LUVs and the exemplary pMPC-LUVs in comparison with the free antimicrobial agents SMX, SMX/EA and EA, and the results are presented in FIG. 11A. Normalized pyocyanin concentrations were measured by HPLC after a 4-hour incubation period with one dose of each treatment.

Results clearly show the inhibitory effect of the exemplary pMPC-LUVs loaded with the exemplary antimicrobial agents (SMX and SMX/EA) on pyocyanin production in P. aeruginosa biofilms. The results also demonstrate that the delivery of combined SMX/EA induces a significant reduction in pyocyanin production, with 15% decrease already observed for administered PEG-LUVs loaded with the combination of SMX/EA, and a decrease of up to about 65% in pyocyanin production in the presence of pMPC-LUVs loaded with SMX/EA.

The two compounds were sequentially encapsulated within the liposomes (EA then SMX) using a modified transmembrane gradient approach as described herein (see hereinabove, Remote drug loading and efficiency determination), resulting in a green-tinted sample as seen in the photograph on FIG. 11B.

The final SMX:EA:lipid molar ratios was 0.22:0.6:1, a 60% lower antibiotic dosage compared to SMX-only liposomes, where maximal encapsulation was at a SMX:lipid molar ratio of 0.55:1.0.

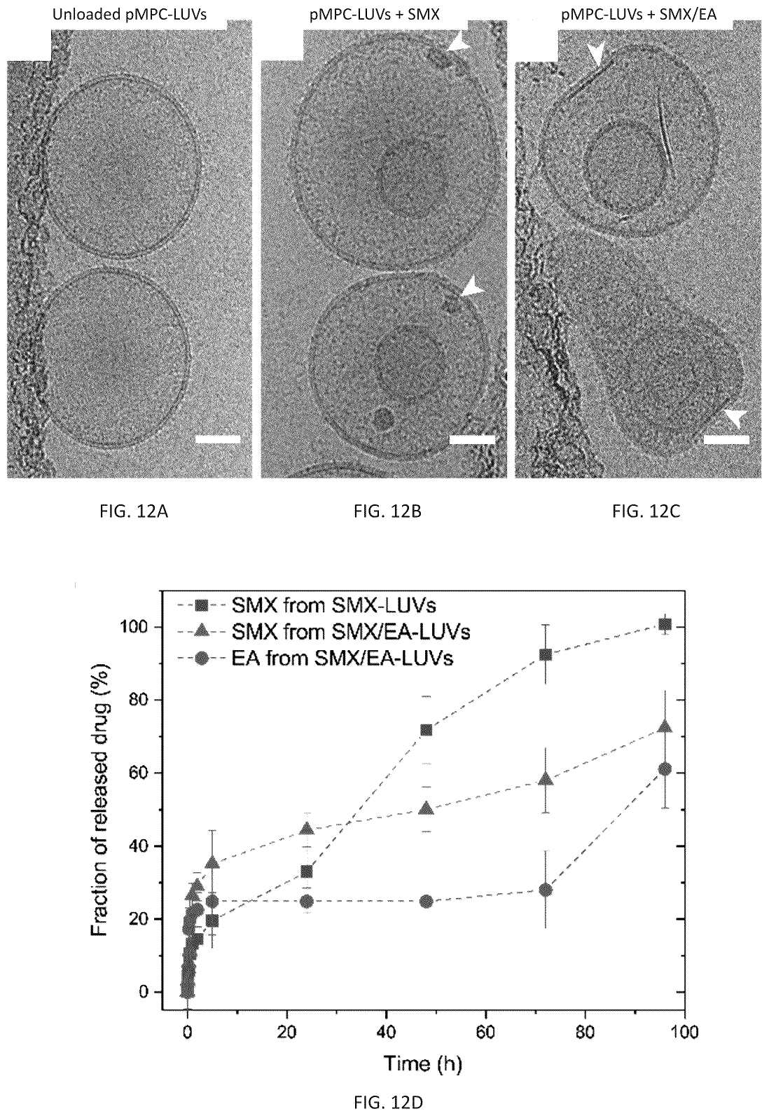

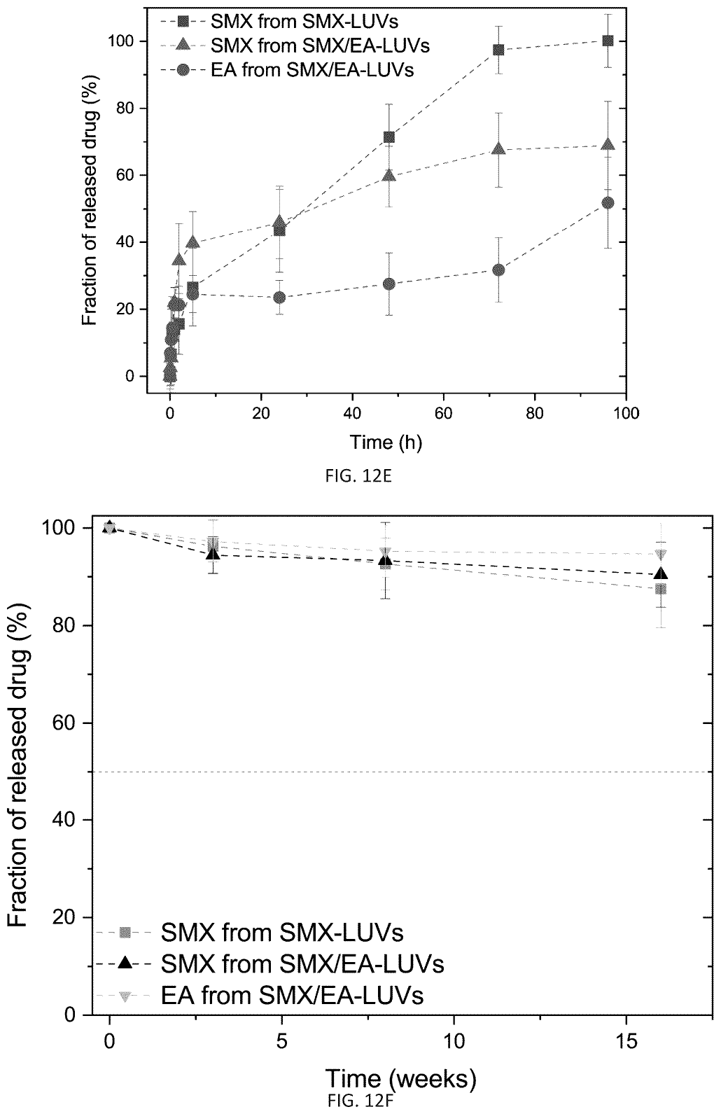

Encapsulation was visually confirmed using cryo-TEM imaging, as seen in FIGS. 12A-C and 13A-C. The images reveal the presence of both SMX and EA inside liposomes as darker interior, with long elongated crystals altering the vesicles' shape.

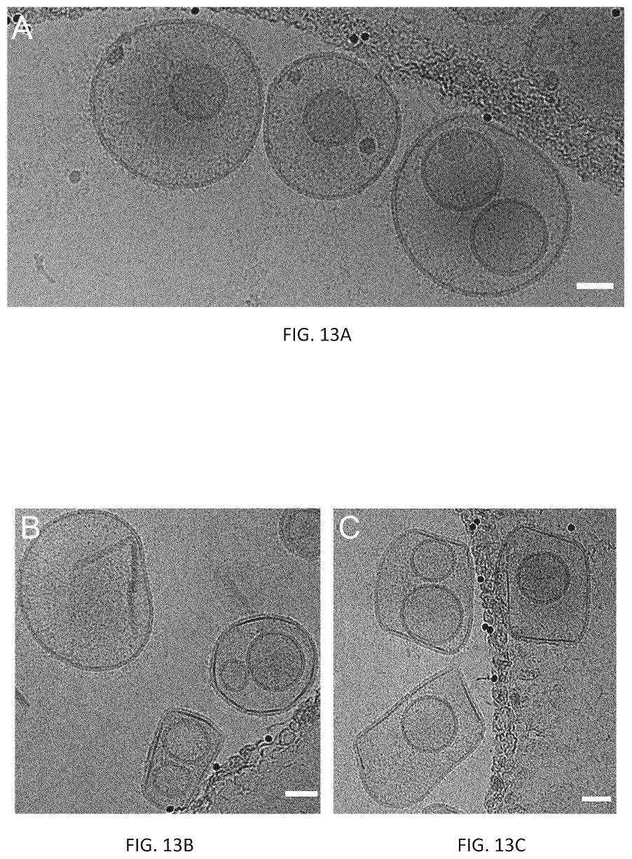

Conversely, in FIG. 12A, unloaded liposomes appear unaltered, whereas SMX-only-loaded LUVs in FIGS. 12B and 13A lack crystal features and present only darker structures.

Despite shape alteration, the overall SMX/EA-loaded LUV size distribution displays a negligible 10 nm diameter increase upon loading, independently of surface functionalization (as can be seen on Table 4), and comparable for both loading configurations. Table 4 hereinbelow shows the Zeta potential and hydrodynamic diameter of pMPC-LUVs and PEG-LUVs loaded with the exemplary antimicrobial agents, sulfamethoxazole (SMX:lipid at a molar ratio of 0.55:1.0) alone or co-loaded with ellagic acid (SMX:EA:lipid at a molar ratio of 0.22:0.6:1.0).

TABLE 4

| | SMX | | SMX/EA | |
|---|---|---|---|---|
| | Diameter | | Diameter | |
| Sample | (nm) | PDI | (nm) | PDI |
| HSPC (50%)/ | 181.5 | 0.05 | 189.4 | 0.19 |
| cholesterol (40%)/ | | | | |
| stearylamine (5%)/ | | | | |
| pMPC (5 kDa, 5%) | | | | |
| HSPC (50%)/ | 164.9 | 0.05 | 171.4 | 0.12 |
| cholesterol (40%)/ | | | | |
| stearylamine (5%)/ | | | | |
| PEG (5 kDa, 5%) | | | | |

Values are shown as averages over 3 samples with 10 runs each.

Release profiles of the loaded pMPCylated liposomes at physiological conditions (37° C., pH 7.2) show that SMX alone is fully (100%) released over 100 hours, while SMX or EA from co-loaded vesicles has slower release kinetics, with only about 60% of the total encapsulated drug being released over the same period. PEGylated liposomes have similar release profiles, as seen in FIG. 12E.

As seen in FIG. 12F, the stability of the encapsulated compounds show long-term storage (up to 3 months) at 4° C., retaining 90±5% of the drug.

### Example 7

### Eradication of Bacterial Biofilms

Eradication of P. aeruginosa PA14 biofilms was examined following two 4 hours of consecutive administrations of drug-loaded liposomes using a MBEC-based resazurin assay (see herein, Determination of biofilm eradication by a combination of MBEC™ and resazurin assays), and results are shown in FIG. 14A.

Incubation with control non-loaded liposomes shows no significant changes in cell viability compared to untreated cells. Delivery of co-loaded SMX-EA- or SMX-loaded LUVs results in a clear loss of cell viability, with larger loss for co-loaded carriers. pMPCylated-LUV carriers achieved 60-65% viability-reduction compared to non-treated bacteria, significantly better than PEGylated-LUVs (45-50% reduction) and even more significant compared to free-drug treatment (30-45% viability reduction).

The improved effect with SMX-EA compared to SMX-alone is especially notable, as the actual encapsulated amount of antibiotic in the co-loaded LUVs is reduced by 60%, indicating that EA strongly enhances SMX effectiveness, as also seen in FIGS. 14B-C when converted to colony-forming units (CFU) as described herein (see General bacterial growth conditions).

To complement results from resazurin spectra, which are dependent on bacterial metabolism and report only live bacteria, the fraction of dead bacteria in the biofilm population was directly imaged and quantified following treatment with the different liposomal configurations and with free antibiotics.

Visualization of PA14 biofilms via LIVE/DEAD™ fluorescence staining and confocal microscopy is shown in FIGS. 15A-B and approximately mirrors the findings obtained by MBEC™ and resazurin assay, as seen in FIG. 14A. In FIG. 15A, untreated biofilms incubated with empty liposomes show a predominant green (live) signal, whereas treatment with free SMX produces a significant increase in the number of red loci, i.e. dead cells. The results were quantified and are presented in FIG. 15B.

As can be seen, treatment with two doses of either SMX/EA- or SMX- loaded LUVs results in more dead bacteria, for both functionalizations, with pMPCylation displaying the highest biofilm eradication, up to 85%, compared to PEGylation (50-60% dead cells) or to free drugs (50-55%).

In order to demonstrate the ability of pMPCylated liposomes to readily deliver their cargo even to an air-exposed biofilm, commonly found in wound and lung infections as well as non-biological surfaces [Attinger and Wolcott, Adv Wound Care 2012, 1 (3), 127-132; and Francolini and Donelli, Fems Immunol Medical Microbiol 2010, 59 (3), 227-238], the LIVE/DEAD™ confocal microscopy assay was then employed on biofilm colonies grown on a solid medium.

P. Aeruginosa (PA14 strain) biofilm colonies were grown and embedded in paraffin post-treatment with liposomes vehicles, to enable coronal sectioning and subsequent microscopy imaging (see herein, Paraffin-embedded thin sectioning and LIVE/DEAD staining for fluorescence imaging). The results are presented in FIGS. 16A-C.

Treatment with unloaded LUVs shows a physiological fraction of coexisting dead (red signal) and live bacteria (green signal).

FIG. 16B shows that the addition of SMX-loaded liposomes followed by a 4-hour incubation period, results in a clear increase of dead bacteria (red) at the point of injection, indicating that pMPC-functionalized liposomes comprising stearylamine may also be used to eradicate air-exposed biofilms.

Image quantification (see herein, Data analysis) is presented in FIGS. 16A-E and confirms the antimicrobial effects along a gradient from the injection point and shows that pMPCylated LUVs achieve a 2.5-fold higher eradication of bacteria relative to PEGylated LUVs, highlighting their higher delivery efficiency even in challenging conditions such as administration to air-exposed biofilms.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method for treating biofilm in a subject in need thereof, the method comprising administering to the subject a liposome comprising:
(a) at least one bilayer-forming lipid;
(b) a polymeric compound having the general formula I: 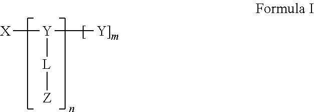
wherein:
m is zero or a positive integer;
n is an integer which is at least 1, wherein when X does not comprise a phosphate group, n is at least 2;
X is a lipid moiety;
Y is a backbone unit which forms a polymeric backbone;
L is absent or is a linking moiety; and
Z has the general formula II: 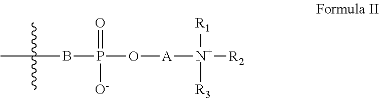
wherein:
A is a substituted or unsubstituted hydrocarbon;
B is an oxygen atom or is absent; and
R₁-R₃ are each independently selected from the group consisting of hydrogen, alkyl, cycloalkyl, heteroalicyclic, aryl and heteroaryl;
c) a positively-charged lipidic agent, incorporated within a lipid bilayer and/or on a surface of the liposome; and
(d) a therapeutically active agent, bound to a surface of the liposome and/or within a lipid bilayer and/or core of the liposome.

2. The method of claim 1, further comprising a sterol, bound to a surface of the liposome and/or within a lipid bilayer and/or core of the liposome.

3. The method of claim 1, wherein said positively-charged lipidic agent comprises a hydrocarbon chain of from 4 to 30 carbon atoms in length, substituted and/or terminated by at least one substituent that is positively charged at physiological conditions.

4. The method of claim 3, wherein said substituent is or comprises an amine.

5. The method of claim 1, wherein said positively-charged lipidic agent is stearyl amine.

6. The method of claim 1, wherein:
a molar ratio of said bilayer-forming lipid and said positively-charged lipidic agent is in a range of from 1:1 to 100:1, or from 1:1 to 50:1; and/or
a molar ratio of said bilayer-forming lipid and said polymeric compound is in a range of from 1:1 to 100:1, or from 5:1 to 50:1; and/or
a molar ratio of said positively-charged lipidic agent and said polymeric compound is in a range of from 10:1 to 1:10.

7. The method of claim 1, wherein Y is a substituted or unsubstituted alkylene unit having the formula —CR₄R₅—CR₆D—, wherein:
when Y is a backbone unit which is not attached to said L or said Z, D is R₇; and when Y is a backbone unit which is attached to said L or said Z, D is a covalent bond or a linking group attaching Y to said L or said Z, said linking group being selected from the group consisting of —O, —S—, alkylene, arylene, sulfinyl, sulfonyl, phosphate, phosphonyl, phosphinyl, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, and amino; and
R₄-R₇ are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azide, azo, phosphate, phosphonyl, phosphinyl, oxo, carbonyl, thiocarbonyl, urea, thiourea, O-carbamyl, N-carbamyl, O-thiocarbamyl, N-thiocarbamyl, C-amido, N-amido, C-carboxy, O-carboxy, sulfonamido, and amino.

8. The method of claim 7, wherein L is a substituted or unsubstituted hydrocarbon from 1 to 10 carbon atoms in length.

9. The method of claim 1, wherein:
B is an oxygen atom; and/or
A is a substituted or unsubstituted hydrocarbon from 1 to 4 carbon atoms in length; and/or
R₁-R₃ are each independently hydrogen or C₁₋₄-alkyl.

10. The method of claim 1, wherein X has the general formula III: 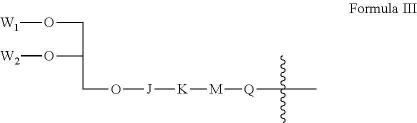
wherein:
W₁ and W₂ are each independently selected from the group consisting of hydrogen, alkyl, alkenyl, alkynyl and acyl, wherein at least one of W₁ and W₂ is alkyl, alkenyl, alkynyl or acyl, being from 10 to 30 carbon atoms in length;
J is —P(═O)(OH)—O— or absent;
K is a substituted or unsubstituted hydrocarbon from 1 to 10 carbon atoms in length;
M is a linking group selected from the group consisting of —O—, —S—, amino, sulfinyl, sulfonyl, phosphate, phosphonyl, phosphinyl, carbonyl, thiocarbonyl, urea, thiourea, carbamyl, thiocarbamyl, amido, carboxy, and sulfonamide, or absent; and
Q is a substituted or unsubstituted hydrocarbon from 1 to 10 carbon atoms in length, or absent,
wherein when M is absent, Q is also absent.

11. The method of claim 10, wherein J is —P(═O)(OH)—O— and K is selected from the group consisting of an ethanolamine moiety, a serine moiety, a glycerol moiety and an inositol moiety.

12. The method of claim 11, wherein M is amido and/or Q is dimethylmethylene (—C(CH₃)₂—).

13. The method of claim 1, wherein said therapeutically active agent is an antimicrobial agent effective in treating said biofilm.

14. The method of claim 1, wherein said biofilm is a bacterial biofilm.

15. The method of claim 1, comprising at least two therapeutically active agents, each independently bound to a surface of the liposome and/or within a lipid bilayer and/or core of the liposome.

16. The method of claim 15, wherein said at least two therapeutically active agents act in synergy.

17. The method of claim 1, wherein the liposome is formulated as part of a pharmaceutical composition, which further comprises a pharmaceutically acceptable carrier.

18. The method of claim 17, wherein the pharmaceutical composition comprises a plurality of said liposome.

19. The method of claim 18, wherein in at least one portion of said plurality of liposomes each liposome comprises a first therapeutically active agent bound to a surface of the liposome and/or within a lipid bilayer and/or core of the liposome, and in at least one another portion of said plurality of liposomes, each liposome comprises a second therapeutically active agent bound to a surface of the liposome and/or within a lipid bilayer and/or core of the liposome, said first and second therapeutically active agents being different from one another.

20. The method of claim 19, wherein said first and second therapeutically active agents act in synergy.
